(19) United States
(12) Patent Application Publication
(10) Pub. No.: US 2025/0082781 A1
(43) Pub. Date: Mar. 13, 2025

(54) METHODS OF LOWERING BLOOD GLUCOSE AND TREATING TYPE 2 DIABETES BY ACTIVATION OF PDE4D3

(71) Applicant: Salk Institute for Biological Studies, La Jolla, CA (US)

(72) Inventors: Ronald M. Evans, La Jolla, CA (US); Michael Downes, San Diego, CA (US); Annette R. Atkins, San Diego, CA (US); Gencer Sancar, San Diego, CA (US); Sihao Liu, San Diego, CA (US)

(73) Assignee: Salk Institute for Biological Studies, La Jolla, CA (US)

(21) Appl. No.: 18/725,478

(22) PCT Filed: Dec. 28, 2022
(86) PCT No.: PCT/US2022/082455

§ 371 Date: Jun. 28, 2024

Related U.S. Application Data
(60) Provisional application No. 63/294,563, filed on Dec. 29, 2021.

(51) Int. Cl. A61K 48/00; A61K 31/192; A61K 31/195; A61K 31/421; A61K 31/422; A61K 31/426; A61K 31/427; A61K 31/4439; A61K 31/64; A61K 38/28; A61K 38/46; A61K 47/64; A61K 47/68; A61P 3/10

(52) U.S. Cl. CPC A61K 48/0058; A61K 31/192; A61K 31/195; A61K 31/421; A61K 31/422; A61K 31/426; A61K 31/427; A61K 31/4439; A61K 31/64; A61K 38/28; A61K 38/465; A61K 47/64; A61K 47/6849; A61K 48/0033; A61P 3/10

(57) ABSTRACT

Methods of lowering blood glucose and treating Type 2 diabetes in a subject by increasing expression or activity of phosphodiesterase 4D isoform 3 (PDE4D3) in adipocytes of the subject are described. In some instances, expression of PDE4D3 in adipocytes is increased by administering a vector that expresses PDE4D3 specifically in adipocytes, or via gene editing by introduction of a PDE4D3-encoding nucleic acid into adipocytes. Use of small molecule activators of PDE4D3 that are targeted to adipocytes is also described.

### CROSS REFERENCE TO RELATED APPLICATIONS

This application claims the benefit of U.S. Provisional Application No. 63/294,563, filed Dec. 29, 2021, which is herein incorporated by reference in its entirety.

### FIELD

This disclosure concerns methods of reducing blood glucose and treating type 2 diabetes (T2D) in a subject by administering an effective amount of an agent that increases expression or activity of phosphodiesterase 4D isoform 3 (PDE4D3) in adipocytes of the subject.

### INCORPORATION OF ELECTRONIC SEQUENCE LISTING

The electronic sequence listing, submitted herewith as an XML file named 7158-107397-02 SL.xml (59,387 bytes), created on Dec. 27, 2022, is herein incorporated by reference in its entirety.

### BACKGROUND

Chronic hyperglycemia and dyslipidemia are hallmarks of Type 2 diabetes mellitus (T2DM) attributed to the failure of insulin to appropriately suppress hepatic glucose production and adipose lipolysis. Moreover, unregulated lipolysis leads to the aberrant accumulation of free fatty acids (FFAs) in peripheral metabolic tissues including liver, muscle, and pancreatic islets, further exacerbating disease severity (Saponaro et al., 2015; Sears and Perry, 2015). Physiologically, adipose lipolysis is regulated, in part, by opposing hormonal stimuli that control cyclic adenosine monophosphate (cAMP) levels and protein kinase A (PKA) activity (Bartness et al., 2014; Duncan et al., 2007). Pro-lipolytic hormones (e.g., glucagon, growth hormone, thyroid hormone, cortisol, catecholamines) elevate cellular cAMP levels to drive PKA phosphorylation of key lipolytic proteins including perilipin and hormone-sensitive lipase (HSL). Conversely, insulin remains the only known anti-lipolytic hormone, acting via phosphodiesterase 3B (PDE3B) to suppress cAMP levels and inhibit PKA activity (Kitamura et al., 1999; Strålfors and Honnor, 1989; Young et al., 2006). Phosphodiesterases (PDEs) catalyze the conversion of cAMP to AMP. Eleven PDE families (PDE1-PDE11), each encompassing multiple isoforms, have been described (Azevedo et al., 2014). In adipose tissue in particular, different PDE4 isoforms have been implicated in the regulation of the cAMP/PKA pathway, however their contributions to lipolysis are not known (Baeza-Raja et al., 2016; Grønning et al., 2006; Zhang et al., 2009). Half of the PDE activity in adipocytes is attributed to PDE4A-D, where the isoform-specific N-terminal domains regulate protein-protein interactions and subcellular localization (Houslay and Adams, 2003; Young et al., 2006). Consistent with this, PDE4 inhibitors enhance lipolysis, particularly when PDE3 activity is inhibited (Dipilato et al., 2015; Grønning et al., 2006; Kraynik et al., 2013; Snyder et al., 2005). Mice deficient in PDE4A, PDE4B and PDE4D genes have been generated (Jin and Conti, 2002; Jin et al., 2005; Jin et al., 1999). Loss of PDE4A and PDE4B in adipocytes led to increased cAMP levels without affecting lipolysis (Grønning et al., 2006; Zhang et al., 2009). In contrast, adipocyte PDE4D expression is induced by insulin and synthetic catecholamines, and lower PDE4D levels are associated with increased β-adrenergic signaling, implicating a potential role in lipolysis (Jang et al., 2020; Oknianska et al., 2007).

Fibroblast growth factor 1 (FGF1) has an established role in adaptive adipose remodeling (Jonker et al., 2012; Wang et al., 2020). Mice lacking FGF1 develop a more aggressive diabetic phenotype in response to a dietary challenge (high fat diet, HFD) that is, in part, attributed to a failure to appropriately remodel adipose tissue. FGF1 expression in adipose tissue is controlled by peroxisome proliferator-activated receptor gamma (PPARγ) and is robustly induced in the fed state and upon HFD feeding (Choi et al., 2016; Jonker et al., 2012). In addition, peripheral delivery of FGF1 rapidly lowers blood glucose levels in diabetic mouse models in an adipose FGF receptor (FGFR) 1-dependent manner (Suh et al., 2014).

### SUMMARY

It is disclosed herein that FGF1 suppresses adipose lipolysis and the anti-lipolytic activities of FGF1 are required for acute glucose lowering. Further disclosed is the discovery that these FGF1 activities are mediated by the activation of phosphodiesterase 4D isoform 3 (PDE4D3) in adipose tissue. In view of these findings, the present disclosure provides methods of lowering blood glucose and/or treating Type 2 diabetes in a subject by increasing expression or activity of PDE4D3.

Provided herein are methods of reducing blood glucose in a subject and/or treating type 2 diabetes in a subject. In some aspects, the method includes administering to the subject a therapeutically effective amount of an agent that increases expression or activity of PDE4D3 in adipocytes of the subject.

In some examples, the agent that increases expression or activity of PDE4D3 includes a nucleic acid molecule encoding a PDE4D3 protein, such as a vector that includes the nucleic acid molecule operably linked to an adipocyte-specific promoter. In other examples, the agent is a nucleic acid molecule encoding the PDE4D3 protein that is introduced into adipocytes using a gene editing method.

In some examples, the agent that increases expression or activity of PDE4D3 is a small molecule activator of PDE4D3, such as an N-substituted-2-(3-aryl-1H-1,2,4-triazol-1-yl) acetamide (e.g., MR-L2). In specific examples, the small molecule activator of PDE4D3 is conjugated to an antibody that specifically binds fibroblast growth factor receptor 1b (FGFR1b), or is conjugated to an incretin, such as gastric inhibitory peptide (GIP) or glucagon-like peptide-1 (GLP-1).

In some aspects, the method further includes administering to the subject a therapeutically effective amount of a mature fibroblast growth factor 1 (FGF1) protein or a modified mature FGF1 protein, such as a modified mature FGF1 protein that has reduced mitogenicity and/or increased stability compared to native FGF1 protein, and/or selectively binds FGFR1b. In some examples, the method further includes administering a therapeutically effective amount of an additional therapeutic agent, such as an anti-diabetic agent.

Also provided are agents that can be used in such methods, such as a nucleic acid molecule encoding a PDE4D3 protein operably linked to an adipocyte-specific promoter, and vectors (such as plasmids or viral vectors) that include such. In one example, the agent is a small molecule activator of PDE4D3 conjugated to an antibody that specifically binds fibroblast growth factor receptor 1b (FGFR1b). In one example, the agent is a small molecule activator of PDE4D3 conjugated to an incretin, such as GIP or GLP-1. Compositions that include such agents are also provided, for example which may further include a pharmaceutically acceptable carrier such as water, saline, or a buffer.

The foregoing and other objects and features of the disclosure will become more apparent from the following detailed description, which proceeds with reference to the accompanying figures.

### BRIEF DESCRIPTION OF THE DRAWINGS

FIGS. 1A-1I: FGF1 suppresses adipose lipolysis. (FIG. 1A) Ex-vivo lipolysis in gonadal white adipose tissue (gWAT) explants from wildtype (F1WT) and FGF1 KO (FIKO) mice 6 h after refeeding. Data are represented as mean±SEM (n=4, *p<0.05). (FIG. 1B) Lipolysis in mouse SVF-derived adipocytes measured by the cumulative release of free fatty acids (FFAs) into the media over 4 h. Cells were pretreated with vehicle (PBS) or FGF1 (100 ng/ml) for 10 min prior to the induction of lipolysis with 1 nM isoproterenol (ISO). Data are represented as mean±SEM (n=3, *p<0.05, **p<0.01). (FIG. 1C) Dose response of FGF1-induced suppression of lipolysis in 3T3-L1 adipocytes. Cells were pretreated with the indicated doses of FGF1 for 10 min prior to the induction of lipolysis with 100 nM isoproterenol (ISO), and the cumulative release of FFAs over 4 h was measured. Data are represented as mean±SEM (n=3, **p<0.01, ***p<0.001). (FIG. 1D) Serum FFA levels in overnight fasted adR1WT and adR1KO DIO mice 30 min after vehicle (PBS) or FGF1 (0.5 mg/kg) injection. Data are represented as mean±SEM (adR1WT vehicle n=5, FGF1 n=5; adR1KO vehicle n=4, FGF1 n=4 *p<0.05). (FIG. 1E) Ex-vivo lipolysis in gWAT explants from overnight fasted adR1WT and adR1KO DIO mice 2 h after vehicle (PBS) or FGF1 (0.5 mg/kg) injection. Data are represented as mean±SEM (n=5 per group, *p<0.05). (FIG. 1F) 3H-labeled oleic acid turnover in chow-fed adR1WT and adR1KO mice. Overnight fasted mice were injected with vehicle (PBS) or FGF1 (0.5 mg/kg, s.c.) 6 hours prior to the portal vein infusion of 3H-labeled oleic acid. Plasma radioactivity was measured by scintillation counting and normalized to t=1 min. Fractional oleic acid turnover rate was calculated by linear regression of natural log transformed data (adR1WT vehicle n=5, FGF1 n=4; adR1KO vehicle n=4, FGF1 n=5). Data are represented as mean±SEM (*p<0.05). (FIG. 1G) Ad lib fed blood glucose levels in vehicle (PBS) and FGF1 injected (0.5 mg/kg) ob/ob mice with and without co-administration of the adipose triglyceride lipase (ATGL) inhibitor atglistatin (120 mg/kg p.o.) (Veh, n=5; FGF1, n=4; ATGLi, n=5; ATGLi+FGF1, n=6). Data are represented as mean±SEM. (*p<0.05). (FIG. 1H) Western blots of total and S660 phosphorylated HSL (pHSL) in 3T3-L1 adipocytes 10 min after vehicle or FGF1 (100 ng/ml) treatment. Quantification of pHSL-S660 normalized to total HSL is shown in the right panel. Data are represented as mean±SEM (n=4, *p<0.05). (FIG. 1I) Western blots of total and S660 phosphorylated HSL (pHSL) in gWAT from chow-fed C57BL/6J mice 30 min after vehicle (PBS), FGF1 (0.5 mg/kg) or insulin (1 U/kg) injection. Quantification of pHSL-S660 normalized to total HSL (right panel). (Veh, FGF1, n=5; insulin, n=4). Data are represented as mean±SEM (**p<0.01, ***p<0.001). See also FIG. 7.

FIGS. 2A-2F: FGF1 suppresses hepatic glucose production (HGP) in an adipose FGFR1 dependent manner. (FIG. 2A) Pyruvate tolerance test (PTT, left panel) and glycerol tolerance test (Glycerol TT, right panel) in overnight fasted ob/ob mice 2 h after vehicle (PBS) or FGF (0.5 mg/kg) injection. Data are represented as mean±SEM (n=5 per group; * p<0.05, **p<0.01, #p<0.001). (FIG. 2B) PTTs in adR1WT and adR1KO DIO mice, as described in FIG. 2A. Data are represented as mean±SEM (n=4 per group, *p<0.05, **p<0.01). (FIG. 2C) Heatmap of hepatic metabolites in ob/ob mice 2 h after vehicle (PBS) or FGF1 (0.5 mg/kg) injection. DHAP, dihydroxyacetone phosphate; G6P, glucose 6-phosphate; F6P, fructose 6-phosphate; GIP, glucose 1-phosphate; OAA, oxaloacetate; PG, phosphoglycerate; PEP, phosphoenolpyruvate. Vehicle n=5, FGF1 n=6, *p<0.05. (FIG. 2D) Hepatic G6P, F6P and 2-phosphoglycerate (2-PG) levels in HFD-fed adR1WT and adR1KO mice 6 h after vehicle (PBS) or FGF1 (0.5 mg/kg) injection. adR1WT Veh n=8, FGF1=7; adR1KO veh n=6, FGF1=6. Data are represented as mean±SEM (*p<0.05). (FIG. 2E) Hepatic acetyl-CoA levels (left panel) and pyruvate carboxylase (PC) activity (right panel) normalized by protein content in mice described in FIG. 2D (*p<0.05). (FIG. 2F) Basal and glucose-clamped levels of endogenous glucose production (EGP), glucose infusion rate (GIR), and glucose disposal rate (GDR) in ob/ob mice after one week of vehicle (PBS) or FGF1 (0.5 mg/kg q.o.d.) injections, measured during a hyperinsulinemic clamp. Vehicle n=9, FGF1 n=8. Data are represented as mean±SEM (*p<0.05). See also FIG. 8

FIGS. 3A-3H: FGF1 suppression of lipolysis is dependent on PDE4 activity. (FIG. 3A) Kinetics of isoproterenol (ISO, 100 nM) induced cAMP levels in 3T3-L1 adipocytes pretreated with vehicle (PBS) or FGF1 (100 ng/mg) for 15 min, as measured by ELISA. Data are represented as mean±SEM (n=4, *p<0.05, **p<0.01). (FIG. 3B) 3T3-L1 adipocyte lipolysis after vehicle (PBS) or FGF1 (100 ng/ml) treatment in the presence or absence of PDE4 inhibitors (roflumilast, 2 μM and cilomulast, 10 μM). PBS or FGF1 was added 15 min prior to isoproterenol (100 nM) stimulation for 4 h. Data are represented as mean±SEM (n=4, ***p<0.001). (FIG. 3C) cAMP levels in 3T3-L1 adipocytes, pretreated for 15 min with vehicle (PBS) or FGF1 (100 ng/ml) with or without PDE4 inhibitor (roflumilast, 2 μM), 30 min after isoproterenol treatment (100 nM). Normalized GFP fluorescence from cAMP biosensor. Data are presented as mean±SEM (n=12, *p<0.05). (FIG. 3D) Lipolysis in gWAT explants from overnight-fasted DIO mice pretreated with the PDE4 inhibitor (roflumilast, 5 mg/kg p.o.) 1 h prior to vehicle (PBS) or FGF1 (0.5 mg/kg) injection. Mice were sacrificed 2 h later. Data are represented as mean±SEM (n=6, *p<0.05). (FIG. 3E) Kinetics of isoproterenol (ISO, 100 nM) induced perilipin-GFP and HSL-mCherry co-localization in 3T3-L1 adipocytes pretreated for 15 min with vehicle (PBS) or FGF1 (100 ng/ml). Effects of PDE4 (roflumilast, 2 μM; middle panel) and PDE3 (cilostamide, 10 μM; right panel) inhibitors on co-localization. Data are represented as mean±SEM (n=12, *p<0.05, **p<0.01). (FIG. 3F) Lipolysis in 3T3-L1 adipocytes infected with an adipose specific AAV (adAAV) expressing GFP or PDE4D3. Data are represented as mean±SEM (n=7, ***p<0.001). (FIG. 3G) cAMP levels in 3T3-L1 adipocytes infected with adAAVs expressing GFP or PDE4D3 30 min after isoproterenol treatment (100 nM), measured using the Green-down biosensor. Data are represented as mean±SEM (n=14, **p<0.01). (FIG. 3H) Kinetics of isoproterenol-induced perilipin-GFP and HSL-mCherry co-localization in 3T3-L1 adipocytes infected with adAAVs expressing PDE4D3 or control vector without an open reading frame. Data are represented as mean±SEM (n=19, **p<0.01). See also FIG. 9.

FIGS. 4A-4G: FGF1-induced suppression of lipolysis and blood glucose is dependent on PDE4D. (FIG. 4A) Blood glucose levels in ad lib fed DIO mice after administration of vehicle (30% captisol) or the PDE4 inhibitor roflumilast (5 mg/kg p.o.). Data are represented as mean±SEM (n=5 per group, ***p<0.001). (FIG. 4B) Serum FFA levels 1 h after the injection of the vehicle or the PDE4 inhibitor in the mice described in FIG. 4A. (FIG. 4C) ad lib fed blood glucose levels in DIO mice injected with vehicle (PBS) or FGF1 (0.5 mg/kg) in the absence (left panel) or presence (right panel) of the PDE4 inhibitor roflumilast (5 mg/kg). Mice were fasted after the 0 h time point. Data are represented as mean±SEM (Cntrl Veh n=7, Cntrl FGF1 n=6, iPDE4 Veh n=7, iPDE4 FGF1 n=8 per arm, **p<0.01, ***p<0.001). (FIG. 4D) Basal and isoproterenol-stimulated (ISO, 1 μM) lipolysis in gWAT explants from overnight fasted, chow-fed control and PDE4D KO mice. Data are represented as mean±SEM (n=4 per group, **p<0.01). (FIG. 4E) Isoproterenol-induced lipolysis (1 nM) in SVF-derived adipocytes from control and PDE4D KO mouse. Cells were pretreated with vehicle (PBS) or FGF1 (100 ng/ml) for 10 min prior to isoproterenol addition. Data are represented as mean±SEM (n=4 per treatment, *p<0.05). (FIG. 4F) ad lib fed blood glucose levels in control and PDE4D KO DIO mice after vehicle (PBS) or FGF1 (0.5 mg/kg) injection. Mice were fasted after the injection until the 2 h time point, then food was returned. Data are represented as mean±SEM (n=6 per group, *p<0.05, ***p<0.001). (FIG. 4G) ad lib fed blood glucose levels after FGF1 injection (0.5 mg/kg) in PDE4D KO DIO mice 4 weeks after treatment with adAAVs driving the expression of PDE4D3 or GFP. Data are represented as mean±SEM (n=7 per group, *p<0.05). See also FIG. 10.

FIGS. 5A-5M: PDE4D3-S44 phosphorylation is required for the metabolic effects of PDE4D3. (FIG. 5A) Representative Western blot among three independent experiments showing the temporal changes in isoproterenol-induced PDE4D phosphorylation in 3T3-L1 adipocytes pretreated for 15 min with vehicle (PBS) or FGF1 (100 ng/ml). The bracket indicates the phosphorylated, slower migrating PDE4D fraction whereas the arrow points to the hypo-phosphorylated form. Quantification of the phospho-band to total is shown below. (FIG. 5B) Western blots of PDE4D phosphorylation in gWAT from overnight-fasted chow-fed C57BL/6J mice 30 min after vehicle (PBS) or FGF1 (0.5 mg/kg) injection. Quantification of the phospho-band to total is shown on the right. Data are represented as mean±SEM (n=4/arm, #p<0.001). (FIG. 5C) Scheme of mouse PDE4D3 domains and known PKA phosphorylation sites. The conservation of phosphorylation sites between mouse, rat, and human PDE4D3 is shown below (SEQ ID NOs: 33-35). (FIG. 5D) Lipolysis in 3T3-L1 adipocytes infected with adAAVs expressing GFP (control), PDE4D3 (4D3), PDE4D3 S44A, or PDE4D3 S85A. Data are represented as mean±SEM (n=3 per treatment group, ***p<0.001). (FIG. 5E) Representative Western blot from two-independent experiments showing PDE4D3 expression in 3T3-L1 adipocytes infected with adAAVs expressing wildtype, S44A, S85A, or S44A/S85A PDE4D3 30 min after treatment with isoproterenol (1 μM) and the PDE4 inhibitor roflumilast (4 μM). The arrow indicates hypo-phosphorylated, and the bracket indicates phosphorylated PDE4D3. Quantification of the phospho-band to total is shown below. (FIG. 5F) Representative Western blot from three independent experiments showing PDE4D3 levels after 30 min isoproterenol treatment (100 nM) of 3T3-L1 adipocytes infected with adAAVs expressing GFP, wildtype PDE4D3 or PDE4D3 S44A with or without 15 min FGF1 pre-treatment (100 ng/ml) (low exposure-upper panel, high exposure-lower panel). Brackets indicate phosphorylated PDE4D3. Quantification of the phospho-band to total is shown below. (FIG. 5G) Western blots of S44 phosphorylated (upper panel) and total PDE4D (lower panel) in 3T3-L1 adipocytes infected with adAAV PDE4D3 after indicated treatments (FGF1, 10 min pretreatment at 100 ng/ml; ISO, 100 nM isoproterenol for 30 min; or FGF1 pre-treatment and 30 min ISO treatment). Quantification of pS44/total PDE4D is shown in FIG. 11E. (FIG. 5H) Isoproterenol-induced (1 nM) lipolysis in SVF-derived adipocytes from PDE4D KO mouse infected with adAAVs expressing WT or S44A PDE4D3 pretreated with vehicle (PBS) of FGF1 (100 ng/ml) for 10 min. Data are represented as mean±SEM (n=4 per treatment, *p<0.05). (FIG. 5I) Western blots showing PDE4D-S44 phosphorylation in 3T3-L1 adipocytes 15 min after vehicle (PBS) or FGF1 (100 ng/ml) treatment. Cells were pretreated with the PI3K inhibitor wortmannin (5 μM) or DMSO 30 min before vehicle or FGF1 treatment. Quantification of pS44/total PDE4D is shown in FIG. 11H. (FIG. 5J) ad lib fed blood glucose levels in ob/ob mice injected with adAAVs expressing GFP (n=9), PDE4D3 (n=8) or PDE4D3 S44A (n=8). Data are represented as mean±SEM (*p<0.05, **p<0.01). (FIG. 5K) ad lib fed serum free fatty acid levels of ob/ob mice described in FIG. 5J. Data are represented as mean±SEM (n=8 per arm; **p<0.01). (FIG. 5L) ad lib fed blood glucose levels of the mice described in FIG. 5J after FGF1 (0.5 mg/kg) injection. Mice were fasted after the injection until the 4 h time point, then food was returned. Data are represented as mean±SEM (adAAV GFP and adAAV PDE4D3, n=7; adAAV PDE4D3 S44A, n=5; ***p<0.001). (FIG. 5M) Western blots of S44 phosphorylated (upper panel) and total (lower panel) PDE4D in overnight-fasted and 4 h refed mice maintained on chow and HFD. Quantification of pS44 levels are normalized to total PDE4D levels. Data are represented as mean±SEM (*p<0.05, **p<0.01). See also FIG. 11.

FIG. 6: Model of FGF1-induced suppression of lipolysis and HGP. Distinct FGF1 signaling parallels insulin-induced suppression of adipose lipolysis and hepatic glucose production (HGP). FGF1/FGFR1 signaling in adipocytes activates PDE4D to decrease cAMP levels and thereby PKA activity. Reduced PKA activity attenuates HSL phosphorylation/translocation to suppress lipolysis. FGF1-induced suppression of lipolysis reduces hepatic glucose production (HGP) through the allosteric regulation of pyruvate carboxylase.

FIGS. 7A-7L: Suppression of adipose lipolysis by FGF1/FGFR1 signaling. (FIG. 7A) Blood glucose levels in adR1WT (vehicle n=5; FGF1 n=5) and adR1KO DIO mice (vehicle n=5; FGF1 n=6) upon FGF1 injection (0.5 mg/kg). Mice were fasted after the injection until the 2 h time point, then food was returned. Data are represented as mean±SEM (**p<0.01, ***p<0.001). (FIG. 7B) Serum FFA levels (left panel) and insulin levels (right panel) of chow-fed F1WT (n=5) and FIKO (n=4) mice after 6 h refeeding. Data are represented as mean±SEM (*p<0.05). (FIG. 7C) Schematic representation of fat transplantation experiments described in FIG. 7D. (FIG. 7D) Ex-vivo adipose lipolysis assay in endogenous or transplanted gWAT explants of 6 h refed F1WT or FIKO mice. Data are represented as mean±SEM (n=4, *p<0.05, **p<0.01). (FIG. 7E) Ex-vivo lipolysis in gWAT from Fgf1f1/f1 (adFGF1WT) and adipose specific FGF1 KO mice (Fgf1f1/f1 mice crossed to adiponectin Cre; adFGF1KO) that were over-night fasted and 6 h refed. Data are represented as mean±SEM (n=4, adFGF1WT; n=6, adFGF1KO **p<0.01). (FIG. 7F) Lipolysis assay in human SVF-derived adipocytes. FGF1 (100 ng/ml) was added into the culture 10 min before the induction of lipolysis by ISO (1 nM) for 4 h. Data are represented as mean±SEM (n=3, *p<0.05). (FIG. 7G) Lipolysis assay in 3T3-L1 adipocytes after vehicle (PBS) or FGF1 treatment (100 ng/ml)±the FGFR1 inhibitor PD 173074 (1 μM). Data are represented as mean±SEM (n=4 per treatment, **p<0.01). (FIG. 7H) Ex-vivo lipolysis in iWAT explants from overnight fasted adR1WT and adR1KO DIO mice 2 h after vehicle (PBS) or FGF1 (0.5 mg/kg) injection. Data are represented as mean±SEM (n=5 per group, *p<0.05). (FIG. 7I) Ex-vivo lipolysis in gWAT and BAT explants from overnight fasted chow-fed mice that were injected with FGF1 (0.5 mg/kg). Six h later tissues were collected and ex-vivo lipolysis experiment was performed. Data are represented as mean±SEM (n=5 per arm, *p<0.05). (FIG. 7J) VO₂ consumption and RER of ob mice after FGF1 injection as measured by metabolic cages. Arrows indicate the time of FGF1/Veh injection. Data are represented as mean±SEM (n=4/arm). (FIG. 7K) Representative Western blot from two-independent experiments showing the kinetics of the pHSL-S660 and total HSL levels in 3T3-L1 adipocytes after isoproterenol (100 nM) treatment. Cells were treated with vehicle, FGF1, or insulin 10 min before the addition of isoproterenol. Quantification of pHSL-660 levels normalized to total HSL is shown below. (FIG. 7L) Lipolysis assay in 3T3-L1 adipocytes treated with vehicle (PBS), FGF1 (100 ng/ml), or insulin (100 nM). Data are represented as mean±SEM (n=9 per treatment, ***p<0.001).

FIGS. 8A-8E: FGF1 decreases hepatic glucose production via controlling metabolic flux to gluconeogenesis. (FIG. 8A) Pyruvate tolerance test (PTT) (left panel) and glycerol tolerance test (Glycerol TT) of overnight-fasted DIO mice 2 h after vehicle (n=6) or FGF1 (n=7) injection (0.5 mg/kg). Data are represented as mean±SEM (*p<0.05, **p<0.01, ***p<0.001). (FIG. 8B) Schematic diagram showing the steps in hepatic glucose production. The metabolites labeled in color were determined by targeted metabolomic profiling and down-regulated in the liver after FGF1 injection. (FIG. 8C) Relative levels of the key metabolites involved in the citric acid cycle as measured by targeted metabolomic profiling. Data are represented as mean±SEM (vehicle n=5; FGF1 n=6). (FIG. 8D) Pyruvate carboxylase, PEPCK and G6Pase levels in livers of overnight fasted ob/ob mice 2 hours after injection of vehicle or FGF1, as measured by Western blot. Protein levels are normalized to actin for quantification (n=6/arm). (FIG. 8E) β-oxidation assay of hepatic mitochondria from ob/ob mice 14 h after Vehicle (PBS) or FGF1 injection. Mitochondria were incubated with BSA-conjugated ¹⁴C palmitate, and released ¹⁴C CO₂ was measured by scintillation counting. Data are represented as mean±SEM (n=5/arm).

FIGS. 9A-9Q: Antilipolytic role of FGF1 is dependent on PDE4. (FIG. 9A) Lipolysis assay in 3T3-L1 adipocytes after vehicle (PBS) or FGF1 (100 ng/ml) treatment±the PI3K inhibitor wortmannin (5 μM). Data are presented as mean±SEM (n=3, ***p<0.001). (FIG. 9B) CRE-Luc cAMP reporter activity in 3T3-L1 adipocytes after 6 h of ISO treatment with 10 min vehicle (PBS) or FGF1 (100 ng/ml) pre-treatment. The CRE-Luc signal was normalized to Renilla luciferase signal. Data are represented as mean±SEM (n=per treatment, **p<0.01). (FIG. 9C) 3T3-L1 adipocyte lipolysis after vehicle (PBS) or FGF1 (100 ng/ml) treatment in the presence or absence of the PDE3 inhibitors (cilostamide (10 μM) or quazinone (10 μM)). Data are represented as mean±SEM (n=4). (FIG. 9D) Lipolysis assays in mouse SVF-derived adipocytes after vehicle (PBS) or FGF1 (100 ng/ml) treatment in the presence of the PDE4 inhibitor roflumilast (2 μM). PBS or FGF1 was added 15 min prior to isoproterenol (1 nM) stimulation for 4 h. Data are represented as mean±SEM (n=4, *p<0.05). (FIG. 9E) Lipolysis assays in human SVF-derived adipocytes after vehicle (PBS) or FGF1 (100 ng/ml) treatment in the presence of the PDE4 inhibitor roflumilast (2 μM) or PDE3 inhibitor cilostamide (10 μM). PBS or FGF1 was added 15 min prior to isoproterenol (1 nM) stimulation for 4 h. Data are represented as mean±SEM (n=3, **p<0.01). (FIG. 9F) Normalized GFP signal levels in 3T3-L1 adipocytes infected with control CMV-GFP and treated with ISO (100 nM) along with vehicle (PBS) or FGF1 (100 ng/ml) pre-treatment. Data are represented as mean±SEM (n=15). (FIG. 9G) Lipolysis in iWAT explants from overnight-fasted DIO mice pretreated with the PDE4 inhibitor (roflumilast, 5 mg/kg p.o.) 1 h prior to vehicle (PBS) or FGF1 (0.5 mg/kg) injection. Mice were sacrificed 2 h later. Data are represented as mean±SEM (n=6 per group, *p<0.05). (FIG. 9H) Western blot showing pHSL-S660 and total HSL levels in 3T3-L1 adipocytes after treatment with vehicle (PBS), FGF1 (100 ng/ml), ISO (100 nM), or the PDE4 inhibitor roflumilast (2 μM). Quantification of pHSL-S660 levels normalized to total HSL levels are shown in the bottom panel. Data are represented as mean±SEM (n=3 wo ISO groups, n=3 DMSO with ISO, n=4 iPDE4 with ISO, *p<0.05). (FIG. 91) High-resolution microscopy images of the perilipin-GFP and HSL-mCherry signals in 3T3-L1 adipocytes before and 30 min after ISO treatment (100 nM) without (upper panel) or with FGF1 (100 ng/ml) pre-treatment (lower panel). Live cell imaging was performed with LSM 880 Airyscan microscope at 40× objective. White arrows indicate the localization of HSL-mCherry signal around the lipid droplets (marked by perilipin-GFP) after ISO stimulation. (FIG. 9J) Kinetics of isoproterenol (ISO, 100 nM) induced perilipin-GFP and mCherry co-localization in 3T3-L1 along with vehicle (PBS) or FGF1 (100 ng/ml) pre-treatment. Data are represented as mean±SEM (n=18, *p<0.05). (FIG. 9K) Lipolysis assays in 3T3-L1 adipocytes that are infected with increasing doses of GFP or PDE4D isoforms expressing adenovirus. Data are represented as mean±SEM (n=4-10, *p<0.05, **p<0.01, ***p<0.001). (FIG. 9L) Schematic representation of adipose-specific expression vector (adAAV) design. ITR, inverted-terminal repeats; hAdipoq, human adiponectin enhancer/promoter; BGH, bovine growth hormone poly-A site. * represents miRNA-122 target site to limit liver expression. (FIG. 9M) Brightfield (left panel) and fluorescence (right panel) images of 3T3-L1 pre-adipocytes and adipocytes that were infected with adAAV GFP. (FIG. 9N) PDE4D protein levels in 3T3-L1 pre-adipocytes and adipocytes after infected with adAAV GFP or adAAV PDE4D3. (FIG. 9O) Western blot showing pHSL-S660 and total HSL levels in 3T3-L1 adipocytes that were infected with adAAV GFP or adAAV PDE4D3. The cells were harvested after ISO-stimulated (100 nM) lipolysis. Quantification of pHSL-S660 levels normalized to total HSL levels are shown in the right panel. Data are represented as mean±SEM (n=3, ***p<0.001). (FIG. 9P) Basal and isoproterenol (1 nM) induced lipolysis assay in human SVF-derived adipocytes infected with adAAV GFP or adAAV PDE4D3. Data are presented as mean±SEM (n=3, ***p<0.001). (FIG. 9Q) Western blot showing the pHSL-S660 and total HSL levels in human SVF-derived adipocytes infected with adAAV GFP or adAAV PDE4D3. The cells were harvested after ISO-stimulated lipolysis (1 nM, 4 h). Quantification of pHSL-S660 levels normalized to total HSL levels are shown in the lower panel. Data are represented as mean±SEM (n=3, ***p<0.001).

FIGS. 10A-10F: PDE4 is required for glucose homeostasis in DIO mice. (FIG. 10A) Blood glucose levels in 5 h fasted DIO mice after administration of vehicle (30% captisol) or the PDE4 inhibitor roflumilast (5 mg/kg p.o.). Data are represented as mean±SEM (n=5 per group, ***p<0.001). (FIG. 10B) Serum FFA levels 1 h after the injection of vehicle or PDE4 inhibitor in the mice described in FIG. 10A. (FIG. 10C) Serum insulin levels of DIO mice described in FIG. 4A and FIG. 10A 1 h after vehicle or roflumilast administration. (FIG. 10D) Blood glucose levels of DIO mice before and 2 h after vehicle (PBS) or FGF1 (0.5 mg/kg) injection in the absence (left panel) or presence (right panel) of the PDE3 inhibitor cilostamide (10 mg/kg). The mice were fasted after cilostamide injection and veh/FGF1 was administered 1 h later. Data are represented as mean±SEM (n=5 for Control Veh, n=6 for other groups, **p<0.01, ***p<0.001). (FIG. 10E) Body weight measurements of Control and PDE4D KO mice on HFD. Data are represented as mean±SEM (Control, n=21; PDE4D KO, n=13, ***p<0.001). (FIG. 10F) Insulin tolerance test of DIO Control and DIO PDE4D KO mice. Data are presented as mean±SEM (Control, n=6; PDE4D KO, n=5).

FIGS. 11A-11K: Phosphorylation of PDE4D-S44 is required for both in vitro and in vivo anti-lipolytic function of PDE4D. (FIG. 11A) Western blot showing the PDE4D, pHSL-S660 and total HSL levels in 3T3-L1 adipocytes that are infected with adAAV GFP or adAAV PDE4D3 S44A. (FIG. 11B) Western blot showing the level and phosphorylation status of WT PDE4D3 and PDE4D3-S44A mutant after the treatment of 3T3-L1 adipocytes with ISO and/or the PDE4 inhibitor roflumilast (4 μM). Roflumilast was added 10 min prior to 30 min isoproterenol stimulation. Arrow indicates the phosphorylated PDE4D3. (FIG. 11C) Western blot showing the level and phosphorylation status of PDE4D3 from samples described in FIG. 5E with or without phosphatase treatment (CIP). (FIG. 11D) Western blot showing the PDE4D-pS44 levels (upper panel) and total PDE4D levels in 3T3-L1 adipocytes that were infected with adAAV PDE4D3, adAAV PDE4D3 S44A, adAAV PDE4D3 S85A, or adAAV PDE4D3 S44A S85A. Samples were treated with ISO (1 μM) and the PDE4 inhibitor roflumilast (4 μM) except for the control sample. (FIG. 11E) Quantification of the pS44 levels were normalized to total levels as described in FIG. 5G (n=3/arm). Data are represented as mean±SEM (**p<0.01). (FIG. 11F) Western blots showing the pS44-PDE4D, PDE4D and tubulin levels from gWATs of wild type and PDE4D-mice. (FIG. 11G) PDE activity in 3T3-L1 adipocytes that were infected with adAAV GFP, adAAV PDE4D3 or adAAV PDE4D-S44A mutant (n=6/arm). Data are represented as mean±SEM (***p<0.001). (FIG. 11H) Quantification of the pS44 levels normalized to total levels as described in FIG. 5I (n=3/arm). Data are represented as mean±SEM (***p<0.001). (FIG. 11I) Western blots showing the GFP and tubulin protein levels in various tissues of mice injected with adAAV GFP. Note that expression of GFP is restricted to adipose tissues. (FIG. 11J) Ad lib fed serum insulin levels of ob/ob mice injected with adAAV GFP, adAAV PDE4D3 or adPDE4D3-S44A. Data are represented as mean±SEM (n=8 per group). (FIG. 11K) Overnight fasted blood glucose (left panel) and serum FFA levels (right panel) of ob/ob mice injected with adAAV GFP, adAAV PDE4D3 or adPDE4D3-S44A. Data are represented as mean±SEM. (n=8 per group, * p<0.05, **p<0.01).

FIG. 12 shows the amino acid sequences of four exemplary mutant FGF1 proteins (SL001, SL002, SL003 and SL004, set forth as SEQ ID NOs: 29-32, respectively).

### SEQUENCE LISTING

The nucleotide and amino acid sequences listed in the accompanying sequence listing are shown using standard letter abbreviations for nucleotide bases, and single letter code for amino acids, as defined in 37 C.F.R. 1.822. Only one strand of each nucleic acid sequence is shown, but the complementary strand is understood as included by any reference to the displayed strand. In the accompanying sequence listing:

```
SEQ ID NO: 1 is a nucleic acid sequence encoding human PDE4D3, deposited under GenBank®
Accession No. NM_006203.5).
      1 ggattcaata cttgttgcaa taattgccca cgatagctgc tcaaacaaga gagttggaat
    61 tcatctgtaa aaatcactac atgtaacgta ggagacaaga aaaatattaa tgacagaaga
  121 tctgcgaaca tgatgcacgt gaataatttt ccctttagaa ggcattcctg gatatgtttt
  181 gatgtggaca atggcacatc tgcgggacgg agtcccttgg atcccatgac cagcccagga
  241 tccgggctaa ttctccaagc aaattttgtc cacagtcaac gacgggagtc cttcctgtat
  301 cgatccgaca gcgattatga cctctctcca aagtctatgt cccggaacte ctccattgcc
  361 agtgatatac acggagatga cttgattgtg actccatttg ctcaggtctt ggccagtctg
  421 cgaactgtac gaaacaactt tgctgcatta actaatttgc aagatcgagc acctagcaaa
  481 agatcaccca tgtgcaacca accatccatc aacaaagcca ccataacaga ggaggcctac
  541 cagaaactgg ccagcgagac cctggaggag ctggactggt gtctggacca gctagagacc
  601 ctacagacca ggcactccgt cagtgagatg gcctccaaca agtttaaaag gatgcttaat
  661 cgggagctca cccatctctc tgaaatgagt cggtctggaa atcaagtgtc agagtttata
  721 tcaaacacat tcttagataa gcaacatgaa gtggaaattc cttctccaac tcagaaggaa
  781 aaggagaaaa agaaaagacc aatgtctcag atcagtggag tcaagaaatt gatgcacagc
  841 tctagtctga ctaattcaag tatcccaagg tttggagtta aaactgaaca agaagatgtc
  901 cttgccaagg aactagaaga tgtgaacaaa tggggtcttc atgttttcag aatagcagag
  961 ttgtctggta accggccctt gactgttatc atgcacacca tttttcagga acgggattta
1021 ttaaaaacat ttaaaattcc agtagatact ttaattacat atcttatgac tctcgaagac
1081 cattaccatg ctgatgtggc ctatcacaac aatatccatg ctgcagatgt tgtccagtct
1141 actcatgtgc tattatctac acctgctttg gaggctgtgt ttacagattt ggagattctt
1201 gcagcaattt ttgccagtgc aatacatgat gtagatcatc ctggtgtgtc caatcaattt
1261 ctgatcaata caaactctga acttgccttg atgtacaatg attcctcagt cttagagaac
1321 catcatttgg ctgtgggctt taaattgctt caggaagaaa actgtgacat tttccagaat
1381 ttgaccaaaa aacaaagaca atctttaagg aaaatggtca ttgacatcgt acttgcaaca
1441 gatatgtcaa aacacatgaa tctactggct gatttgaaga ctatggttga aactaagaaa
1501 gtgacaagct ctggagttct tcttcttgat aattattccg ataggattca ggttcttcag
1561 aatatggtgc actgtgcaga tctgagcaac ccaacaaagc ctctccagct gtaccgccag
1621 tggacggacc ggataatgga ggagttcttc cgccaaggag accgagagag ggaacgtggc
1681 atggagataa gccccatgtg tgacaagcac aatgcttccg tggaaaaatc acaggtgggc
1741 ttcatagact atattgttca tcccctctgg gagacatggg cagacctcgt ccaccctgac
1801 gcccaggata ttttggacac tttggaggac aatcgtgaat ggtaccagag cacaatccct
1861 cagagcccct ctcctgcacc tgatgaccca gaggagggcc ggcagggtca aactgagaaa
1921 ttccagtttg aactaacttt agaggaagat ggtgagtcag acacggaaaa ggacagtggc
1981 agtcaagtgg aagaagacac tagctgcagt gactccaaga ctctttgtac tcaagactca
2041 gagtctactg aaattcccct tgatgaacag gttgaagagg aggcagtagg ggaagaagag
2101 gaaagccagc ctgaagcctg tgtcatagat gatcgttctc ctgacacgta acagtgcaaa
2161 aactttcatg cctttttttt ttttaagtag aaaaattgtt tccaaagtgc atgtcacatg
2221 ccacaaccac ggtcacacct cactgtcatc tgccaggacg tttgttgaac aaaactgacc
2281 ttgactactc agtccagcgc tcaggaatat cgtaaccagt tttttcacct ccatgtcatc
2341 cgagcaaggt ggacatcttc acgaacagcg tttttaacaa gatttcagct tggtagagct
2401 gacaaagcag ataaaatcta ctccaaatta ttttcaagag agtgtgactc atcaggcagc
2461 ccaaaagttt attggacttg gggtttctat tcctttttat ttgtttgcaa tattttcaga
2521 agaaaggcat tgcacagagt gaacttaatg gacgaagcaa caaatatgtc aagaacagga
2581 catagcacga atctgttacc agtaggagga ggatgagcca cagaaattgc ataattttct
2641 aatttcaagt cttcctgata catgactgaa tagtgtggtt cagtgagctg cactgacctc
2701 tacattttgt atgatatgta aaacagattt tttgtagagc ttacttttat tattaaatgt
2761 attgaggtat tatatttaaa aaaaactatg ttcagaactt catctgccac tggttatttt
2821 tttctaagga gtaacttgca agttttcagt acaaatctgt gctacactgg ataaaaatct
2881 aatttatgaa ttttacttgc accttatagt tcatagcaat taactgattt gtagtgattc
2941 attgtttgtt ttatatacca atgacttcca tattttaaaa gagaaaaaca actttatgtt
3001 gcaggaaacc ctttttgtaa gtctttatta tttactttgc attttgtttc actctttcca
3061 gataagcaga gttgctcttc accagtgttt ttcttcatgt gcaaagtgac tatttgttct
3121 ataatacttt tatgtgtgtt atatcaaatg tgtcttaagc ttcatgcaaa ctcagtcatc
3181 agttcgtgtt gtctgaagca agtgggagat atataaatac ccagtagcta aaatggtcag
3241 tcttttttag atgttttcct acttagtatc tcctaataac gttttgctgt gtcactagat
3301 gttcatttca caagtgcatg tctttctaat aatccacaca tttcatgctc taataatcca
3361 cacatttcat gctcattttt attgttttta cagccagtta tagtaagaaa aaggtttttc
3421 cccttgtgct gctttataat ttagcgtgtg tctgaacctt atccatgttt gctagatgag
3481 gtcttgtcaa atatatcact accattgtca ccggtgaaaa gaaacaggta gttaagttag
3541 ggttaacatt catttcaacc acgaggttgt atatcatgac tagcttttac tcttggttta
3601 cagagaaaag ttaaacagcc aactaggcag tttttaagaa tattaacaat atattaacaa
3661 acaccaatac aactaatcct atttggtttt aatgatttca ccatgggatt aagaactata
3721 tcaggaacat ccctgagaaa cggttttaag tgtagcaact actcttcctt aatggacagc
3781 cacataacgt gtaggaagtc ctttatcact tatcctcgat ccataagcat atcttgcaga
3841 ggggaactac ttctttaaac acatggaggg aaagaagatg atgccactgg caccagaggg
3901 ttagtactgt gatgcatcct aaaatattta ttatattggt aaaaattctg gttaaataaa
3961 aaattagaga tcactcttgg ctgatttcag caccaggaac tgtattacag ttttagagat
4021 taattcctag tgtttacctg attatagcag ttggcatcat ggggcattta attctgactt
4081 tatccccacg tcagccttaa taaagtcttc tttaccttct ctatgaagac tttaaagccc
4141 aaataatcat ttttcacatt gatattcaag aattgagata gatagaagcc aaagtgggta
4201 tctgacaagt ggaaaatcaa acgtttaaga agaattacaa ctctgaaaag catttatatg
4261 tggaacttct caaggagcct cctggggact ggaaagtaag tcatcagcca ggcaaatgac
4321 tcatgctgaa gagagtcccc atttcagtcc cctgagatct agctgatgct tagatccttt
4381 gaaataaaaa ttatgtcttt ataactctga tcttttacat aaagcagaag aggaatcaac
4441 tagttaattg caaggtttct actctgtttc ctctgtaaag atcagatggt aatctttcaa
4501 ataagaaaaa aataaagacg tatgtttgac caagtagttt cacaagaata tttgggaact
4561 tgtttctttt aattttattt gtccctgagt gaagtctaga aagaaaggta aagagtctag
4621 agtttattcc tctttccaaa acattctcat tcctctcctc cctacactta gtatttcccc
4681 cacagagtgc ctagaatctt aataatgaat aaaataaaaa gcagcaatat gtcattaaca
4741 aatccagacc tgaaagggta aagggtttat aactgcacta ataaagagag gctctttttt
4801 tttcttccag tttgttggtt tttaatggta ccgtgttgta aagataccca ctaatggaca
4861 atcaaattgc agaaaaggct caatatccaa gagacaggga ctaatgcact gtacaatctg
4921 cttatccttg cccttctctc ttgccaaagt gtgcttcaga aatatatact gctttaaaaa
4981 agaataaaag aatatccttt tacaagtggc tttacatttc ctaaaatgcc ataagaaaat
5041 gcaatatctg ggtactgtat ggggaaaaaa atgtccaagt ttgtgtaaaa ccagtgcatt
5101 tcagcttgca agttactgaa cacaataatg ctgttttaat tttgttttat atcagttaaa
5161 attcacaata atgtagatag aacaaattac agacaaggaa agaaaaaact tgaatgaaat
5221 ggattttaca gaaagcttta tgataatttt tgaatgcatt atttattttt tgtgccatgc
5281 attttttttc tcaccaaatg accttacctg taatacagtc ttgtttgtct gtttacaacc
5341 atgtatttat tgcaatgtac atactgtaat gttaattgta aattatctgt tcttattaaa
5401 acatcatccc atgatgggat ggtgttgata tatttggaaa ctcttggtga gagaatgaat
5461 ggtgtgtata catactctgt acatttttct tttctcctgt aatatagtct tgtcacctta
5521 gagcttgttt atggaagatt caagaaaact ataaaatact taaagatata taaatttaaa
5581 aaaacatagc tgcaggtctt tggtcccagg gctgtgcctt aactttaacc aatattttct
5641 tctgttttgc tgcatttgaa aggtaacagt ggagctaggg ctgggcattt tacatccagg
5701 cttttaattg attagaattc tgccaatagg tggattttac aaaaccacag acaacctctg
5761 aaagattctg agaccctttt gagacagaag ctcttaagta cttcttgcca gggagcagca
5821 ctgcatgtgt gatggttgtt tgccatctgt tgatcaggaa ctacttcagc tacttgcatt
5881 tgattatttc cttttttttt ttttttaact cggaaacaca actggggaaa tatattcttt
5941 cccagtgatt ataaacaatc tttttctttt ttttaagtcc ttttggcttc tagagctcat
6001 aggaaaatgg acttgatttg aaattggagc cagagtttac tcgtgttggt tatctattca
6061 tcagcttcct gacatgttaa gagaatacat taaagagaaa atactgtttt ttaatcctaa
6121 aatttttctt ccactaagat aaaccaaatg tccttacata tatgtaaacc catctattta
6181 aacgcaaagg tgggttgatg tcagtttaca tagcagaaag cattcactat cctctaagat
6241 ttgtttctgc aaaactttca ttgctttaga attttaaaat ttcaccttgt acaatggcca
6301 gcccctaaag caggaaacat ttataatgga ttatatggaa acatcctccc agtacttgcc
6361 cagcccttga atcatgtggc ttttcagtga aaggaaagat tctttttcta ggaaaaatga
6421 gcctatttta ttttatttta ttttattttt tgacacaaac tgtagatttt agcagccctg
6481 gcccaaagga atttgattac ttttgtttta aacagtacaa aggggacact ataattacaa
6541 aaacatcctt aactgatttg agttgttttt atttctttgg atatattttc agagtggtaa
6601 attgtgtgtg agaattacaa atgattattc ttttagtggt ttcttagcct ctcttacagc
6661 ccacggggat agtactgtac atcaatacct tcatatgaaa tttttatatg caatgaaaat
6721 aaaagcatgg gttgattctg cctatttatg actcaatctt ttacaaataa aagattattc
6781 attttaaatt atagttcaat cagcatgtct cttaggatac tgaacgtggt tgaaatgaaa
6841 ggatagtgac atcataagtt agtactgata ttcataacca aataaagcca acttgagtaa
6901 ttttgctaca ttaaaaatta ccaaaattac ttagatggcc tataagatta agcatggtgt
6961 tttctaagca agctttgaaa ggggccttcc atacttactt aattgaatat tctgggatat
7021 tgaaaattat tcagatactt gacaattatt tttggttacc tactccgcaa actacaaagt
7081 tttaaggact caacaataag ttaatgagac acagtgtttg ctttcatgga gcttacagtc
7141 tggaggggac aaaggcttaa acaatactca tataattata tatgtgatca gtacaatgaa
7201 ggagctcagt ggggtaaata agcaggaacc tgaacttgat ctgttccgga gggccacaga
7261 aggcttcctt gaggccttga gaaagtgatt tgcatctgag ttctgaagga ttgtaagagg
7321 taactaggga aaaagttgac aggaagagga aggggatcca gacaagaaac atttgcaaag
7381 atcttgaggc ataaatgagc ttgagacatc tggagaaact gaggaaaagt gagagagtag
7441 gcagggcctg gagccgcaga gccattgcta accatcctgt gtgagatatc ccccattctg
7501 tagctttatt ctcataaccc tgctcaattt tctttataac acttctcaca gatttatata
7561 cgtgtttgtt tttgttatct gtctctccca ccagaccaca gctccatgag agcaaggtct
7621 ttgcttacca atatatcact agcacttaaa actatgcctg gtacacagta ggttcttaat
7681 atgtgttgaa tatagccatc aaattgatat tggatataat tcaatctgat aagatatttt
7741 gagatattaa agagttttta acttgatacc ataaaaa
SEQ ID NO: 2 is the amino acid sequence of human PDE4D3 deposited under GenBank®
Accession No. NP_006194.2.
    1 mmhvnnfpfr rhswicfdvd ngtsagrspl dpmtspgsgl ilqanfvhsq rresflyrsd
  61 sdydlspksm srnssiasdi hgddlivtpf aqvlaslrtv rnnfaaltnl qdrapskrsp
121 mcnqpsinka titeeayqkl asetleeldw cldqletlqt rhsvsemasn kfkrmlnrel
181 thlsemsrsg nqvsefisnt fldkqhevei psptqkekek kkrpmsqisg vkklmhsssl
241 tnssiprfgv kteqedvlak eledvnkwgl hvfriaelsg nrpltvimht ifqerdllkt
301 fkipvdtlit ylmtledhyh advayhnnih aadvvqsthv llstpaleav ftdleilaai
361 fasaihdvdh pgvsnqflin tnselalmyn dssvlenhhl avgfkllqee ncdifqnltk
421 kqrqslrkmv idivlatdms khmnlladlk tmvetkkvts sgvllldnys driqvlqnmv
481 hcadlsnptk plqlyrqwtd rimeeffrqg drerergmei spmcdkhnas veksqvgfid
541 yivhplwetw adlvhpdaqd ildtlednre wyqstipqsp spapddpeeg rqgqtekfqf
601 eltleedges dtekdsgsqv eedtscsdsk tlctqdsest eipldeqvee eavgeeeesq
661 peacviddrs pdt
SEQ ID NOs: 3-19 are primer sequences (see Table 1).
SEQ ID NO: 20 is the amino acid sequence of a PDE4D3-S44 peptide
(FRRHpSWISFDVDNGTSAGR).
SEQ ID NO: 21 is an exemplary human FGFR1b nucleic acid sequence deposited under
GenBank® Accession No. FJ809917.
      1 agatgcaggg gcgcaaacgc caaaggagac caggctgtag gaagagaagg gcagagcgcc
    61 ggacagctcg gcccgctccc cgtcctttgg ggccgcggct ggggaactac aaggcccagc
  121 aggcagctgc agggggcgga ggcggaggag ggaccagcgc gggtgggagt gagagagcga
  181 gccctcgcgc cccgccggcg catagcgctc ggagcgctct tgcggccaca ggcgcggcgt
  241 cctcggcggc gggcggcagc tagcgggagc cgggacgccg gtgcagccgc agcgcgcgga
  301 ggaacccggg tgtgccggga gctgggcggc cacgtccgga cgggaccgag acccctcgta
  361 gcgcattgcg gcgacctcgc cttccccggc cgcgagcgcg ccgctgcttg aaaagccgcg
  421 gaacccaagg acttttctcc ggtccgagct cggggcgccc cgcagggcgc acggtacccg
  481 tgctgcagtc gggcacgccg cggcgccggg gcctccgcag ggcgatggag cccggtctgc
  541 aaggaaagtg aggcgccgcc gctgcgttct ggaggagggg ggcacaaggt ctggagaccc
  601 cgggtggcgg acgggagccc tccccccgcc ccgcctccgg ggcaccagct ccggctccat
  661 tgttcccgcc cgggctggag gcgccgagca ccgagcgccg ccgggagtcg agcgccggcc
  721 gcggagctct tgcgaccccg ccaggacccg aacagagccc gggggcggcg ggccggagcc
  781 ggggacgcgg gcacacgccc gctcgcacaa gccacggcgg actctcccga ggcggaacct
  841 ccacgccgag cgagggtcag tttgaaaagg aggatcgagc tcactgtgga gtatccatgg
  901 agatgtggag ccttgtcacc aacctctaac tgcagaactg ggatgtggag ctggaagtgc
  961 ctcctcttct gggctgtgct ggtcacagcc acactctgca ccgctaggcc gtccccgacc
1021 ttgcctgaac aagcccagcc ctggggagcc cctgtggaag tggagtcctt cctggtccac
1081 cccggtgacc tgctgcagct tcgctgtcgg ctgcgggacg atgtgcagag catcaactgg
1141 ctgcgggacg gggtgcagct ggcggaaagc aaccgcaccc gcatcacagg ggaggaggtg
1201 gaggtgcagg actccgtgcc cgcagactcc ggcctctatg cttgcgtaac cagcagcccc
1261 tcgggcagtg acaccaccta cttctccgtc aatgtttcag ttcccataga tgctctcccc
1321 tcctcggagg atgatgatga tgatgatgac tcctcttcag aggagaaaga aacagataac
1381 accaaaccaa accccgtagc tccatattgg acatccccag aaaagatgga aaagaaattg
1441 catgcagtgc cggctgccaa gacagtgaag ttcaaatgcc cttccagtgg gaccccaaac
1501 cccacactgc gctggttgaa aaatggcaaa gaattcaaac ctgaccacag aattggaggc
1561 tacaaggtcc gttatgccac ctggagcatc ataatggact ctgtggtgcc ctctgacaag
1621 ggcaactaca cctgcattgt ggagaatgag tacggcagca tcaaccacac ataccagctg
1681 gatgtcgtgg agcggtcccc tcaccggccc atcctgcaag cagggttgcc cgccaacaaa
1741 acagtggccc tgggtagcaa cgtggagttc atgtgtaagg tgtacagtga cccgcagccg
1801 cacatccagt ggctaaagca catcgaggtg aatgggagca agattggccc agacaacctg
1861 ccttatgtcc agatcttgaa gcattcgggg attaatagct cggatgcgga ggtgctgacc
1921 ctgttcaatg tgacagaggc ccagagcggg gagtatgtgt gtaaggtttc caattatatt
1981 ggtgaagcta accagtctgc gtggctcact gtcaccagac ctgtggcaaa agccctggaa
2041 gagaggccgg cagtgatgac ctcgcccctg tacctggaga tcatcatcta ttgcacaggg
2101 gccttcctca tctcctgcat ggtggggtcg gtcatcgtct acaagatgaa gagtggtacc
2161 aagaagagtg acttccacag ccagatggct gtgcacaagc tggccaagag catccctctg
2221 cgcagacagg taacagtgtc tgctgactcc agtgcatcca tgaactctgg ggttcttctg
2281 gttcggccat cacggctctc ctccagtggg actcccatgc tagcaggggt ctctgagtat
2341 gagcttcccg aagaccctcg ctgggagctg cctcgggaca gactggtctt aggcaaaccc
2401 ctgggagagg gctgctttgg gcaggtggtg ttggcagagg ctatcgggct ggacaaggac
2461 aaacccaacc gtgtgaccaa agtggctgtg aagatgttga agtcggacgc aacagagaaa
2521 gacttgtcag acctgatctc agaaatggag atgatgaaga tgatcgggaa gcataagaat
2581 atcatcaacc tgctgggggc ctgcacgcag gatggtccct tgtatgtcat cgtggagtat
2641 gcctccaagg gcaacctgcg ggagtacctg caggcccgga ggcccccagg gctggaatac
2701 tgctacaacc ccagccacaa cccagaggag cagctctcct ccaaggacct ggtgtcctgc
2761 gcctaccagg tggcccgagg catggagtat ctggcctcca agaagtgcat acaccgagac
2821 ctggcagcca ggaatgtcct ggtgacagag gacaatgtga tgaagatagc agactttggc
2881 ctcgcacggg acattcacca catcgactac tataaaaaga caaccaacgg ccgactgcct
2941 gtgaagtgga tggcacccga ggcattattt gaccggatct acacccacca gagtgatgtg
3001 tggtctttcg gggtgctcct gtgggagatc ttcactctgg gcggctcccc ataccccggt
3061 gtgcctgtgg aggaactttt caagctgctg aaggagggtc accgcatgga caagcccagt
3121 aactgcacca acgagctgta catgatgatg cgggactgct ggcatgcagt gccctcacag
3181 agacccacct tcaagcagct ggtggaagac ctggaccgca tcgtggcctt gacctccaac
3241 caggagtacc tggacctgtc catgcccctg gaccagtact cccccagctt tcccgacacc
3301 cggagctcta cgtgctcctc aggggaggat tccgtcttct ctcatgagcc gctgcccgag
3361 gagccctgcc tgccccgaca cccagcccag cttgccaatg gcggactcaa acgccgctga
3421 ctgccaccca cacgccctcc ccagactcca ccgtcagctg taaccctcac ccacagcccc
3481 tgctgggccc accacctgtc cgtccctgtc ccctttcctg ctggcaggag ccggctgcct
3541 accaggggcc ttcctgtgtg gcctgccttc accccactca gctcacctct ccctccacct
3601 cctctccacc tgctggtgag aggtgcaaag aggcagatct ttgctgccag ccacttcatc
3661 ccctcccaga tgttggacca acacccctcc ctgccaccag gcactgcctg gagggcaggg
3721 agtgggagcc aatgaacagg catgcaagtg agagcttcct gagctttctc ctgtcggttt
3781 ggtctgtttt gccttcaccc ataagcccct cgcactctgg tggcaggtgc cttgtcctca
3841 gggctacagc agtagggagg tcagtgcttc gtgcctcgat tgaaggtgac ctctgcccca
3901 gataggtggt gccagtggct tattaattcc gatactagtt tgctttgctg accaaatgcc
3961 tggtaccaga ggatggtgag gcgaaggcca ggttgggggc agtgttgtgg ccctggggcc
4021 cagccccaaa ctgggggctc tgtatatagc tatgaagaaa acacaaagtg tataaatctg
4081 agtatatatt tacatgtctt tttaaaaggg tcgttaccag agatttaccc atcgggtaag
4141 atgctcctgg tggctgggag gcatcagttg ctatatatta aaaacaaaaa agaaaaaaaa
4201 ggaaaatgtt tttaaaaagg tcatatattt tttgctactt ttgctgtttt atttttttaa
4261 attatgttct aaacctattt tcagtttagg tccctcaata aaaattgctg ctgcttcatt
4321 tatctatggg ctgtatgaaa agggtgggaa tgtccactgg aaagaaggga cacccacggg
4381 ccctggggct aggtctgtcc cgagggcacc gcatgctccc ggcgcaggtt ccttgtaacc
4441 tcttcttcct aggtcctgca cccagacctc acgacgcacc tcctgcctct ccgctgcttt
4501 tggaaagtca gaaaaagaag atgtctgctt cgagggcagg aaccccatcc atgcagtaga
4561 ggcgctgggc agagagtcaa ggcccagcag ccatcgacca tggatggttt cctccaagga
4621 aaccggtggg gttgggctgg ggagggggca cctacctagg aatagccacg gggtagagct
4681 acagtgatta agaggaaagc aagggcgcgg ttgctcacgc ctgtaatccc agcactttgg
4741 gacaccgagg tgggcagatc acttcaggtc aggagtttga gaccagcctg gccaacttag
4801 tgaaacccca tctctactaa aaatgcaaaa attatccagg catggtggca cacgcctgta
4861 atcccagctc cacaggaggc tgaggcagaa tcccttgaag ctgggaggcg gaggttgcag
4921 tgagccgaga ttgcgccatt gcactccagc ctgggcaaca gagaaaacaa aaaggaaaac
4981 aaatgatgaa ggtctgcaga aactgaaacc cagacatgtg tctgccccct ctatgtgggc
5041 atggttttgc cagtgcttct aagtgcagga gaacatgtca cctgaggcta gttttgcatt
5101 caggtccctg gcttcgtttc ttgttggtat gcctccccag atcgtccttc ctgtatccat
5161 gtgaccagac tgtatttgtt gggactgtcg cagatcttgg cttcttacag ttcttcctgt
5221 ccaaactcca tcctgtccct caggaacggg gggaaaattc tccgaatgtt tttggttttt
5281 tggctgcttg gaatttactt ctgccacctg ctggtcatca ctgtcctcac taagtggatt
5341 ctggctcccc cgtacctcat ggctcaaact accactcctc agtcgctata ttaaagctta
5401 tattttgctg gattactgct aaatacaaaa gaaagttcaa tatgttttca tttctgtagg
5461 gaaaatggga ttgctgcttt aaatttctga gctagggatt ttttggcagc tgcagtgttg
5521 gcgactattg taaaattctc tttgtttctc tctgtaaata gcacctgcta acattacaat
5581 ttgtatttat gtttaaagaa ggcatcattt ggtgaacaga actaggaaat gaatttttag
5641 ctcttaaaag catttgcttt gagaccgcac aggagtgtct ttccttgtaa aacagtgatg
5701 ataatttctg ccttggccct accttgaagc aatgttgtgt gaagggatga agaatctaaa
5761 agtcttcata agtccttggg agaggtgcta gaaaaatata aggcactatc ataattacag
5821 tgatgtcctt gctgttacta ctcaaatcac ccacaaattt ccccaaagac tgcgctagct
5881 gtcaaataaa agacagtgaa attgacctga
SEQ ID NO: 22 is an exemplary human FGFR1b protein sequence, deposited under
GenBank® Accession No. ACO38646.1.
MWSWKCLLFWAVLVTATLCTARPSPTLPEQAQPWGAPVEVESFLVHPGDLLQLRCRLRDDVQSIN
WLRDGVQLAESNRTRITGEEVEVQDSVPADSGLYACVTSSPSGSDTTYFSVNVSVPIDALPSSEDDD
DDDDSSSEEKETDNTKPNPVAPYWTSPEKMEKKLHAVPAAKTVKFKCPSSGTPNPTLRWLKNGKE
FKPDHRIGGYKVRYATWSIIMDSVVPSDKGNYTCIVENEYGSINHTYQLDVVERSPHRPILQAGLPA
NKTVALGSNVEFMCKVYSDPQPHIQWLKHIEVNGSKIGPDNLPYVQILKHSGINSSDAEVLTLFNVT
EAQSGEYVCKVSNYIGEANQSAWLTVTRPVAKALEERPAVMTSPLYLEIIIYCTGAFLISCMVGSVI
VYKMKSGTKKSDFHSQMAVHKLAKSIPLRRQVTVSADSSASMNSGVLLVRPSRLSSSGTPMLAGV
SEYELPEDPRWELPRDRLVLGKPLGEGCFGQVVLAEAIGLDKDKPNRVTKVAVKMLKSDATEKDL
SDLISEMEMMKMIGKHKNIINLLGACTQDGPLYVIVEYASKGNLREYLQARRPPGLEYCYNPSHNP
EEQLSSKDLVSCAYQVARGMEYLASKKCIHRDLAARNVLVTEDNVMKIADFGLARDIHHIDYYKK
TTNGRLPVKWMAPEALFDRIYTHQSDVWSFGVLLWEIFTLGGSPYPGVPVEELFKLLKEGHRMDK
PSNCTNELYMMMRDCWHAVPSQRPTFKQLVEDLDRIVALTSNQEYLDLSMPLDQYSPSFPDTRSST
CSSGEDSVFSHEPLPEEPCLPRHPAQLANGGLKRR
SEQ ID NO: 23 is an exemplary human GIP nucleic acid sequence, deposited under GenBank®
Accession No. NM_004123.3.
    1 agcaggctca gaaggtccag aaatcagggg aaggagaccc ctatctgtcc ttcttctgga
  61 agagctggaa aggaagtctg ctcaggaaat aaccttggaa gatggtggcc acgaagacct
121 ttgctctgct gctgctgtcc ctgttcctgg cagtgggact aggagagaag aaagagggtc
181 acttcagcgc tctcccctcc ctgcctgttg gatctcatgc taaggtgage agccctcaac
241 ctcgaggccc caggtacgcg gaagggactt tcatcagtga ctacagtatt gccatggaca
301 agattcacca acaagacttt gtgaactggc tgctggccca aaaggggaag aagaatgact
361 ggaaacacaa catcacccag agggaggctc gggcgctgga gctggccagt caagctaata
421 ggaaggagga ggaggcagtg gagccacaga gctccccagc caagaacccc agcgatgaag
481 atttgctgcg ggacttgctg attcaagagc tgttggcctg cttgctggat cagacaaacc
541 tctgcaggct caggtctcgg tgactctgac cacacccagc tcaggactgg attctgccct
601 tcacttagca cctgcctcag ccccactcca gaatagccaa gagaacccaa accaataaag
661 tttatgctaa gtcgagccca ttgtgaaaat ttattaaaat gactactgag cactaa
SEQ ID NO: 24 is an exemplary human GIP protein sequence, deposited under GenBank®
Accession No. NP_004114.1.
MVATKTFALLLLSLFLAVGLGEKKEGHFSALPSLPVGSHAKVSSPQPRGPRYAEGTFISDYSIAMDK
IHQQDFVNWLLAQKGKKNDWKHNITQREARALELASQANRKEEEAVEPQSSPAKNPSDEDLLRDL
LIQELLACLLDQTNLCRLRSR
SEQ ID NO: 25 is an exemplary GLP-1 nucleic acid sequence (from Heloderma suspectum),
deposited under GenBank® Accession No. EU790959.1.
    1 atgaaaatca tcctgtggct gtgtgttttt gggctgttcc ttgcaacttt attccctatc
  61 agctggcaaa tgcctgttga atctgggttg tcttctgagg attctgcaag ctcagaaagc
121 tttgcttcga agattaagcg acatggtgaa ggaacattta ccagtgactt gtcaaaacag
181 atggaagagg aggcagtgcg gttatttatt gagtggctta agaacggagg accaagtagc
241 ggggcacctc cgccatcggg ttaa
SEQ ID NO: 26 is an exemplary GLP-1 protein sequence.
HGEGTFTSDLSKQMEEEAVRLFIEWLKNGGPSSGAPPPS
SEQ ID NO: 27 is the nucleic acid sequence of an exemplary adiponectin promoter
and enhancer.
ctctttccacatgacggcctttgtggtgggtggcagattgccctgaggcctcgcaaaatgctaggctttcacaatgtcac
tgactgacagccaggcccagcacagtcttggtgtgattgtggggctaaagttattccaccttgtgcaatagctacagcag
ctttaagaattcagggcctttttaacttgccaagccccacaccactccaggaacttccccacaccccagttctcagaatt
catgtgcaaggtctttcctaaatccagggtccaggtcagagagtggaggatgtgctctatttcttacctgattgcagacc
cctctgacagtgctcccttctgaagcactcactgtctgaacgtacacagtctcagactaatcatgcacagtgagcaagac
tgtggtgtgataattggcgtccctgacttattagggcaaatctatggggggggagacctcctggaccactgagcaatta
attcatttacattaggaagtttctccgtcagatgcaggaaaaaaatcttgttttcctgctgtggttttgacttttgcccc
atcttctgttgctgttgtaggaggcaaaataagggtcaaggcctggaaacacaagtgctttgactgaagctccacttggc
ttccgaagcccaagctgggttgtaccaggttccctagggtgcaggctgtgggcaactgccagggacatgtgcctgcccac
cggcctctggccctcactgagttggccaatgggaaatgacaattgtgaggtggggactgcctgcccccgtgagtaccagg
ctgttgaggctgggccatctcctcctcacttccattctgactgcagtctgtggttctgattccataccagaggg
SEQ ID NO: 28 is the nucleic acid sequence of a 4x synthetic miR-122a target sequence
to restrict expression to adipose tissue.
caaacaccattgtcacactccatcaccaaacaccattgtcacactccaattacaaacaccattgtcacactccatcacca
aacaccattgtcacactcca
SEQ ID NO: 29 is the amino acid sequence of FGF1 mutant SL001.
FNLPPGHYKKPKLLYCSDGGHFLRILPDGTVEGTRDRSDQHIQLQLSAESVGEVYIKSTETGQCLAM
DTDGLLYGSQTPNEECLLLERLGENHYNTYISKRHAEKNWFVGLKKSGSCKRGPRTHYGQKAILFL
PLPVSSD
SEQ ID NO: 30 is the amino acid sequence of FGF1 mutant SL002.
FNLPPGNYKEPKPLYCSNGGHFLRILPDGTVDGTRDRSDQHIQPQLSAESVGEVYIKSTETGQYLAM
DTDGLLYGSQTPNEECLFLERLEENHYNTYTSKKHAEKNWFVGLKQNGSCKRGPRTHYGQKAILF
LPLPVSSD
SEQ ID NO: 31 is the amino acid sequence of FGF1 mutant SL003.
FNLPPGNYKKPKLLYCSDRGHFLRILPDGTVDGTRDRSDQHIQLQLSAESVGEVYIKSTETGQYLAM
DTDGLLYGSQTPNEECLFLERLEENHYNTYISKKHAEKNWFVGLKKNGSCERGPRTHYGQKAILFL
PLPVSSD
SEQ ID NO: 32 is the amino acid sequence of FGF1 mutant SL004.
FNLPPGNYKKPKLLYCSNGGHFLRILPDGTVDGTRDRSDQHIQLQLSAESVGEVYIKSTETGQYLA
MDTDGLLYGPQTPNEECLFLERLEENHYNTYISKKHAEKNWFVGLKKNGSCKRGPRTHYGQKAIL
FLPLPVSSD
```

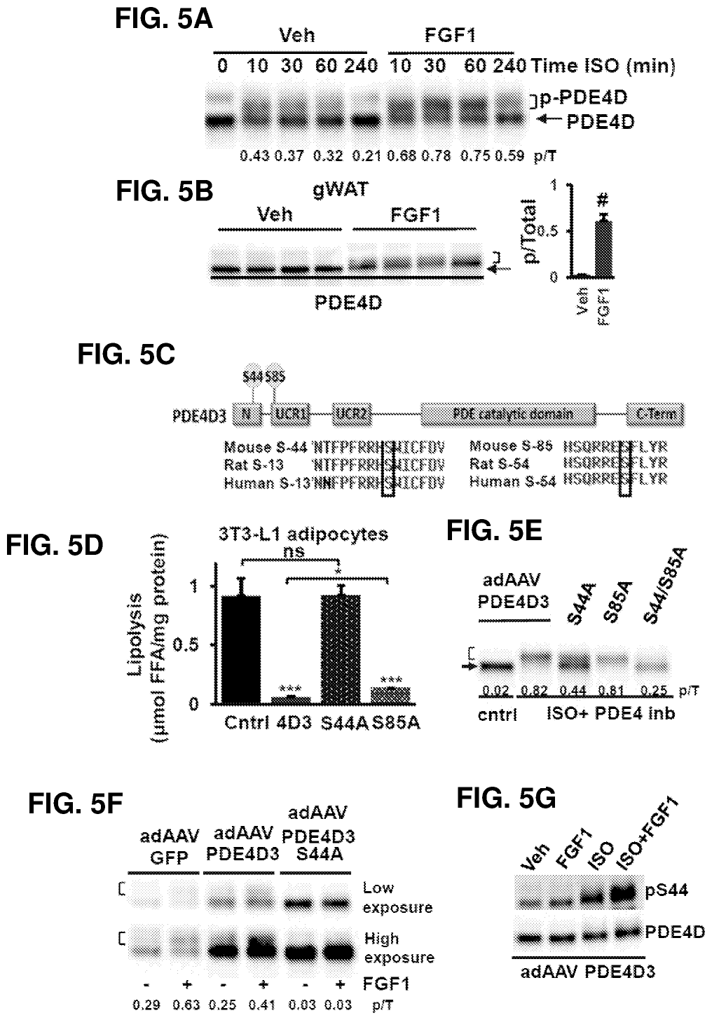

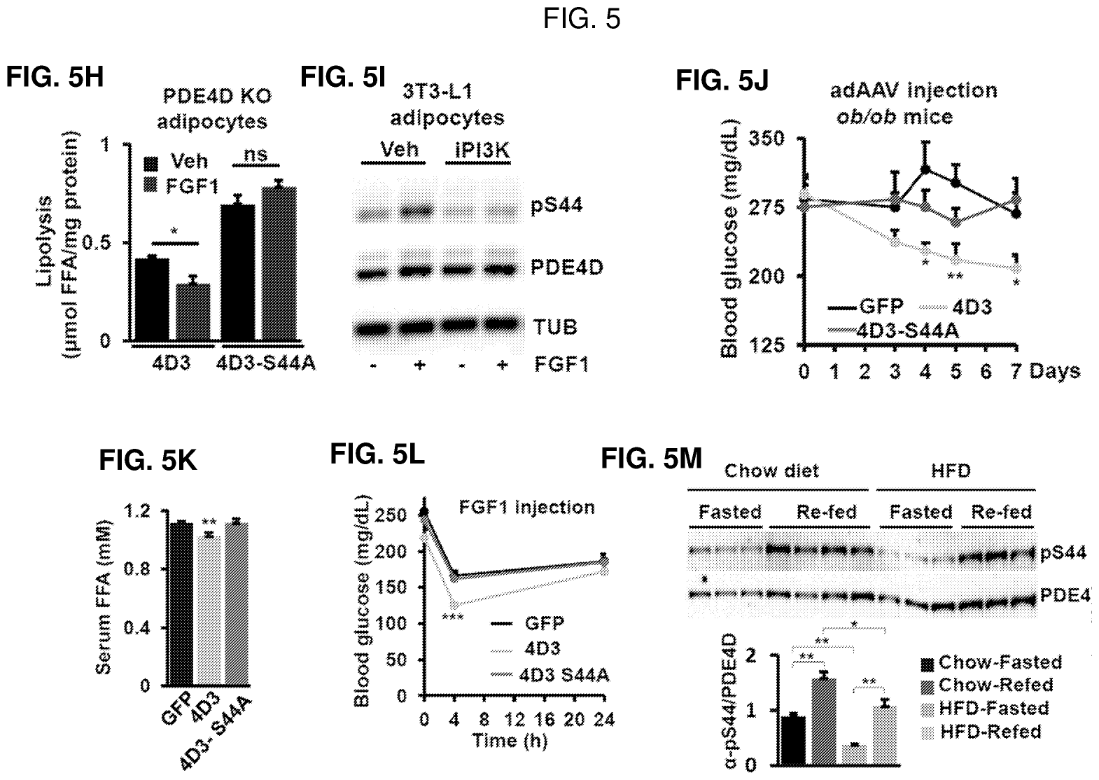

SEQ ID NOs: 33-35 are amino acid sequences of phosphorylation sites in mouse, rat, and human PDE4D3 (see FIG. 5C).

SEQ ID NO: 36 is the amino acid sequence of the QRRES motif.

SEQ ID NO: 37 is the amino acid sequence of the FRRHS motif.

### DETAILED DESCRIPTION

The following explanations of terms and methods are provided to better describe the present disclosure and to guide those of ordinary skill in the art in the practice of the present disclosure. The singular forms “a,” “an,” and “the” refer to one or more than one, unless the context clearly dictates otherwise. For example, the term “comprising a cell” includes single or plural cells and is considered equivalent to the phrase “comprising at least one cell.” The term “or” refers to a single element of stated alternative elements or a combination of two or more elements, unless the context clearly indicates otherwise. As used herein, “comprises” means “includes.” Thus, “comprising A or B,” means “including A, B, or A and B,” without excluding additional elements. Dates of GenBank® Accession Nos. referred to herein are the sequences available at least as early as Dec. 29, 2021. All references, including patents and patent applications, and GenBank® Accession numbers cited herein are incorporated by reference in their entireties.

Unless explained otherwise, all technical and scientific terms used herein have the same meaning as commonly understood to one of ordinary skill in the art to which this disclosure belongs. Although methods and materials similar or equivalent to those described herein can be used in the practice or testing of the present disclosure, suitable methods and materials are described below. The materials, methods, and examples are illustrative only and not intended to be limiting.

In order to facilitate review of the various aspects of the disclosure, the following explanations of specific terms are provided:

Activator: An agent or composition that increases expression of a gene or increases activity of a gene product. In some aspects, an activator of PDE4D3 increases activity of a PDE4D3 protein (for example, increases the phosphodiesterase activity of PDE4D3). In some examples, PDE4D3 activity is increased by at least 10%, at least 20%, at least 25%, at least 30%, at least 40%, at least 50%, at least 60%, at least 75%, at least 80%, at least 90%, at least 95%, at least 100%, at least 200%, at least 300%, at least 400%, or at least 500% relative to a control, such as PDE4D3 activity prior to or in the absence of treatment with the activator. In other aspects, an activator of PDE4D3 increases transcription of a PDE4D3 gene, increases translation of a PDE4D3 mRNA, or decreases degradation of a PDE4D3 mRNA or protein, thereby increasing the level of PDE4D3 protein in the subject or target cell (such as an adipocyte). In some examples, the level of PDE4D3 protein is increased at least 10%, at least 20%, at least 25%, at least 30%, at least 40%, at least 50%, at least 60%, at least 75%, at least 80%, at least 90%, at least 95%, at least 100%, at least 200%, at least 300%, at least 400%, or at least 500% relative to a control, such as the accumulation or level of PDE4D3 protein prior to or in the absence of treatment with the activator.

Adipocyte: A type of cell that is the primary constituent of adipose tissue. These cells specialize in storing energy as fat. In addition, adipocytes are considered to be an endocrine organ that has a significant impact on the metabolism of other tissues, including regulating appetite, insulin sensitivity and immunological responses (Ali et al., Eur J Cell Biol 92 (6-7): 229-236, 2013). Adipocytes, also known as lipocytes and fat cells, are derived from mesenchymal stem cells, which differentiate to adipocytes through adipogenesis.

Administration: To provide or give a subject an agent, such as a small molecule activator of PDE4D3, or a nucleic acid molecule/vector encoding PDE4D3, by any effective route. Exemplary routes of administration include, but are not limited to, oral, injection (such as subcutaneous, intraosseous, intramuscular, intradermal, intraperitoneal, intravenous, intrathecal, and intratumoral), sublingual, rectal, transdermal, intranasal, vaginal and inhalation routes.

Agent: Any substance, compound or drug that is useful for achieving a particular outcome. For example, the agent can be a substance, compound, or drug capable of modulating (such as increasing) expression or activity of PDE4D3. In some aspects, the agent is a small molecule activator of PDE4D3. In other aspects, the agent is a nucleic acid molecule or vector encoding a PDE4D3 protein.

Antibody: A polypeptide ligand comprising at least one variable region that recognizes and binds (such as specifically recognizes and specifically binds) an epitope of an antigen, such as FGFR1b. Mammalian immunoglobulin molecules are composed of a heavy (H) chain and a light (L) chain, each of which has a variable region, termed the variable heavy (V_{H}) region and the variable light (V_{L}) region, respectively. Together, the V_{H} region and the V_{L} region are responsible for binding the antigen recognized by the antibody. There are five main heavy chain classes (or isotypes) of mammalian immunoglobulin, which determine the functional activity of an antibody molecule: IgM, IgD, IgG, IgA and IgE. Antibody isotypes not found in mammals include IgX, IgY, IgW and IgNAR. IgY is the primary antibody produced by birds and reptiles, and has some functionally similar to mammalian IgG and IgE. IgW and IgNAR antibodies are produced by cartilaginous fish, while IgX antibodies are found in amphibians.

Antibody variable regions contain “framework” regions and hypervariable regions, known as “complementarity determining regions” or “CDRs.” The CDRs are primarily responsible for binding to an epitope of an antigen. The framework regions of an antibody serve to position and align the CDRs in three-dimensional space. The amino acid sequence boundaries of a given CDR can be readily determined using any of a number of well-known numbering schemes, including those described by Kabat et al. (Sequences of Proteins of Immunological Interest, U.S. Department of Health and Human Services, 1991; the “Kabat” numbering scheme), Chothia et al. (see Chothia and Lesk, J Mol Biol 196:901-917, 1987; Chothia et al., Nature 342:877, 1989; and Al-Lazikani et al., (JMB 273,927-948, 1997; the “Chothia” numbering scheme), and the ImMunoGene Tics (IMGT) database (see, Lefranc, Nucleic Acids Res 29:207-9, 2001; the “IMGT” numbering scheme). The Kabat and IMGT databases are maintained online.

A “single-domain antibody” refers to an antibody having a single domain (a variable domain) that is capable of specifically binding an antigen, or an epitope of an antigen, in the absence of an additional antibody domain. Single-domain antibodies include, for example, V_{H} domain antibodies, V_{NAR} antibodies, camelid V_{H}H antibodies, and V_{L} domain antibodies. V_{NAR} antibodies are produced by cartilaginous fish, such as nurse sharks, wobbegong sharks, spiny dogfish and bamboo sharks. Camelid V_{H}H antibodies are produced by several species including camel, llama, alpaca, dromedary, and guanaco, which produce heavy chain antibodies that are naturally devoid of light chains.

A “monoclonal antibody” is an antibody produced by a single clone of lymphocytes or by a cell into which the coding sequence of a single antibody has been transfected. Monoclonal antibodies are produced by methods known to those of skill in the art. Monoclonal antibodies include humanized monoclonal antibodies.

A “chimeric antibody” has framework residues from one species, such as human, and CDRs (which generally confer antigen binding) from another species.

A “humanized” antibody is an immunoglobulin including a human framework region and one or more CDRs from a non-human (for example a mouse, rabbit, rat, shark or synthetic) immunoglobulin. The non-human immunoglobulin providing the CDRs is termed a “donor,” and the human immunoglobulin providing the framework is termed an “acceptor.” In one aspect, all CDRs are from the donor immunoglobulin in a humanized immunoglobulin. Constant regions need not be present, but if they are, they must be substantially identical to human immunoglobulin constant regions, i.e., at least about 85-90%, such as about 95% or more identical. Hence, all parts of a humanized immunoglobulin, except possibly the CDRs, are substantially identical to corresponding parts of natural human immunoglobulin sequences. A humanized antibody binds to the same antigen as the donor antibody that provides the CDRs. Humanized or other monoclonal antibodies can have additional conservative amino acid substitutions which have substantially no effect on antigen binding or other immunoglobulin functions.

Blood glucose: Refers to the amount of glucose present in a subject's blood. A normal blood glucose level is generally considered to range from about 80 to about 130 mg/dL (if measured before a meal). If measured within two hours of a meal, blood glucose of less than about 180 mg/dL is considered normal. Higher levels of blood glucose can be indicative of diabetes or pre-diabetes. Blood glucose can be measured, for example, using a continuous glucose monitor (CGM). Blood glucose is also referred to as blood sugar.

Diabetes mellitus: A group of metabolic diseases in which a subject has high blood sugar, either because the pancreas does not produce enough insulin, or because cells do not respond to the insulin that is produced. Type 1 diabetes results from the body's failure to produce insulin. This form has also been called “insulin-dependent diabetes mellitus” (IDDM) or “juvenile diabetes”. Type 2 diabetes (T2D) results from insulin resistance, a condition in which cells fail to use insulin properly, sometimes combined with an absolute insulin deficiency. This form is also called “non-insulin-dependent diabetes mellitus” (NIDDM) or “adult-onset diabetes.” The defective responsiveness of body tissues to insulin is believed to involve the insulin receptor. Diabetes mellitus is characterized by recurrent or persistent hyperglycemia, and in some examples diagnosed by demonstrating any one of:
a. Fasting plasma glucose level ≥7.0 mmol/l (126 mg/dl);
b. Plasma glucose ≥11.1 mmol/l (200 mg/dL) two hours after a 75 g oral glucose load as in a glucose tolerance test;
c. Symptoms of hyperglycemia and casual plasma glucose ≥11.1 mmol/l (200 mg/dl);
d. Glycated hemoglobin (Hb A1C)≥6.5%

Effective amount or therapeutically effective amount: The amount of an agent, such as an activator of PDE4D3 or a nucleic acid molecule encoding PDE4D3, that is sufficient to prevent, treat (including prophylaxis), reduce, and/or ameliorate the symptoms and/or underlying causes of any disorder or disease. In one aspect, a “therapeutically effective amount” is sufficient to reduce or eliminate a symptom of T2D, for example by lowering blood glucose. In some examples, blood glucose is reduced by about 5%, about 10%, about 20%, about 30%, about 40% or about 50% relative to blood glucose prior to treatment.

In some examples, a “therapeutically effective amount” is the amount necessary to increase activity or expression of PDE4D3 at least 10%, at least 20%, at least 25%, at least 30%, at least 40%, at least 50%, at least 60%, at least 75%, at least 80%, at least 90%, at least 95%, at least 100%, at least 200%, at least 300%, at least 400%, or at least 500% or more compared to the activity or expression of a suitable control. In some examples, the therapeutically effective amount is the amount necessary to increase the amount of PDE4D3 protein in a cell (such as an adipocyte) by at least 10%, at least 20%, at least 25%, at least 30%, at least 40%, at least 50%, at least 60%, at least 75%, at least 80%, at least 90%, at least 95%, at least 100%, at least 200%, at least 300%, at least 400%, or at least 500% or more compared to a suitable control.

Fibroblast Growth Factor 1 (FGF1): Includes FGF1 nucleic acid molecules and proteins (e.g., OMIM 13220). FGF1 is a protein that binds to FGF receptors and is also known as the acidic FGF. FGF1 sequences are publicly available, for example from the GenBank® sequence database (e.g., Accession Nos. NP_00791 and NP_034327 provide exemplary FGF1 protein sequences, while Accession Nos. NM_000800 and NM_010197 provide exemplary FGF1 nucleic acid sequences). A skilled person can identify additional FGF1 nucleic acid and protein sequences, including FGF1 variants.

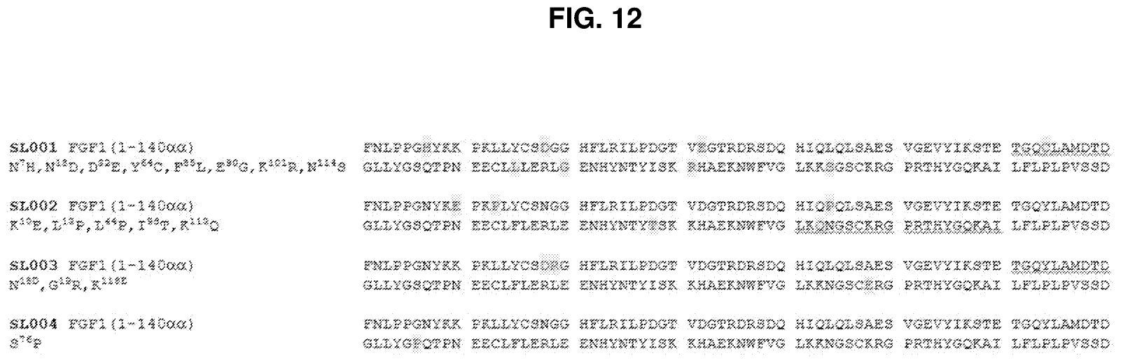

A native FGF1 sequence is one that does not include a mutation that alters the normal activity of the protein. A mature FGF1 refers to an FGF1 peptide or protein product and/or sequence following any post-translational modifications. A mutated FGF1 is a variant of FGF1 with different or altered biological activity, such as reduced mitogenicity. In one example, such a variant includes an N-terminal truncation and/or one or more additional point mutations, such as changes that decrease mitogenicity of FGF1, alter the heparin binding affinity of FGF1, and/or the thermostability of FGF1. Specific exemplary FGF1 mutant proteins are shown in FIG. 12. Additional FGF1 variants are described in WO 2015/061331, WO 2015/061351, WO 2015/061361, WO 2011/130729, WO 2016/172153, WO 2016/172156, WO 2016/172290, WO 2018/026713, WO 2018/112200 and US 2021/0032303.

Fibroblast Growth Factor Receptor 1b (FGFR1b): A member of the FGFR1 family found on the cell surface that has tyrosine kinase activity. The FGFR1 gene can be alternatively spliced to generate distinct mRNAs, which code for two FGFR1 isoforms: FGFR1b and FGFR1c. FGFR1b sequences are publicly available, for example from GenBank® sequence database (e.g., Accession No. ACO38646.1 (SEQ ID NO: 22) provides an exemplary FGFR1b protein sequence, while Accession No. FJ809917 (SEQ ID NO: 21) provides an exemplary FGFR1b nucleic acid sequence). A skilled person can identify additional FGFR1b nucleic acid and protein sequences, including FGFR1b variants.

Gene editing: Refers to methods that enable the specific alteration (such as by deletion, insertion or substitution) of the genome of an organism. Gene editing techniques typically employ nucleases that introduce breaks into DNA strands, which allows for the removal of existing DNA and/or insertion of a desired DNA sequence (such as DNA encoding PDE4D3). Examples include, but are not limited to, clustered regularly interspaced short palindromic repeats (CRISPR)-CRISPR-associated protein 9 (Cas9), transcription activator-like effector nucleases (TALENs), and zinc-finger nucleases (ZFNs) (see, e.g., Fridovich-Keil, “Gene Editing,” Encyclopedia Britannica, Jun. 4, 2019, available online at britannica.com/science/gene-editing; Knott and Doudna, Science 361 (6405): 866-869, 2018; U.S. Pat. No. 10,000,772; and U.S. Publication Nos. 2021/0340568, 2021/0338815, 2021/0292769, 2021/0222171). In one example, RNA (such as PDE4D3 RNA) is edited, for example using a Cas13d protein (e.g., see WO 2019/040664).

Increasing expression or activity of PDE4D3: As used herein, an agent that increases expression or activity of PDE4D3 is a compound that increases the level of PDE4D3 mRNA or PDE4D3 protein in a cell or tissue, or increases one or more activities of the PDE4D3 protein. In some examples, increasing expression of PDE4D3 includes increasing transcription of the PDE4D3 gene, increasing translation of the PDE4D3 mRNA, decreasing degradation of the PDE4D3 protein and/or increasing stability of the PDE4D3 protein, thereby increasing the level of PDE4D3 protein in a subject or a cell (such as an adipocyte) as compared to a suitable control. In other examples, increasing activity of the PDE4D3 protein includes increasing phosphodiesterase activity of PDE4D3, which results in a decrease in intracellular cAMP levels.

In some aspects, expression of PDE4D3 is increased by at least 10%, at least 20%, at least 25%, at least 30%, at least 40%, at least 50%, at least 60%, at least 75%, at least 80%, at least 90%, at least 95%, at least 100%, at least 200%, at least 300%, at least 400%, or at least 500% relative to a control. In specific non-limiting examples, a vector encoding PDE4D3 is used to express PDE4D3 in adipocytes, thereby increasing expression of PDE4D3 by at least 10%, at least 20%, at least 25%, at least 30%, at least 40%, at least 50%, at least 60%, at least 75%, at least 80%, at least 90%, at least 95%, at least 100%, at least 200%, at least 300%, at least 400%, or at least 500% relative to a control. In some aspects, activity of PDE4D3 is increased by at least 10%, at least 20%, at least 25%, at least 30%, at least 40%, at least 50%, at least 60%, at least 75%, at least 80%, least 90%, or at least 95%, at least 100%, at least 200%, at least 300%, at least 400%, or at least 500% relative to a control. In specific non-limiting examples, an activator of PDE4D3 (such as MR-L2) increases phosphodiesterase activity of PDE4D3 by at least 10%, at least 20%, at least 25%, at least 30%, at least 40%, at least 50%, at least 60%, at least 75%, at least 80%, at least 90%, at least 95%, at least 100%, at least 200%, at least 300%, at least 400%, or at least 500% relative to a control.

Incretins: A group of metabolic hormones released following nutrient intake. Incretins stimulate insulin secretion from pancreatic beta cells. Examples of incretin hormones include gastric inhibitory peptide (GIP; NCBI Gene ID 2695) and glucagon-like peptide-1 (GLP-1). The receptors for GIP (GIPR) and GLP-1 (GLP-1R) are expressed in adipose and other types of tissue (Capozzi et al., Endocrine Reviews 39 (5): 719-738, 2018).

Isolated: An “isolated” biological component (such as a PDE4D3 protein, or nucleic acid molecule encoding such) has been substantially separated, produced apart from, or purified away from other biological components in the cell of the organism in which the component naturally occurs, such as other chromosomal and extrachromosomal DNA and RNA, and proteins. Nucleic acid molecules and proteins which have been “isolated” thus include nucleic acids and proteins purified by standard purification methods. The term also embraces nucleic acid molecules and proteins prepared by recombinant expression in a host cell as well as chemically synthesized nucleic acids. A purified or isolated cell, protein, or nucleic acid molecule can be at least 70%, at least 80%, at least 90%, at least 95%, at least 96%, at least 97%, at least 98%, or at least 99% pure.

Linker: A bi-functional molecule that can be used to link two molecules into one contiguous molecule, for example, to link an effector molecule (such as a small molecule or drug) to a targeting molecule (such as an antibody or incretin). The terms “conjugating,” “joining,” “bonding,” or “linking” can refer to making two molecules into one contiguous molecule; for example, linking two polypeptides into one contiguous polypeptide, or covalently attaching an effector molecule or detectable marker to a polypeptide. The linkage can be either by chemical or recombinant means. “Chemical means” refers to a reaction between the antibody moiety and the effector molecule such that there is a covalent bond formed between the two molecules to form one molecule. In some aspects, the linker is an acid-sensitive linker (also known as a “pH-sensitive linker” or “acid-labile linker”). Acid-sensitive linkers include a chemical bond, such as an imine, hydrazone, oxime, amide, acetal, or orthoester bond, that is cleaved under acidic conditions (see, e.g., Zhuo et al., Molecules 25:5649, 2020). In particular examples, the acid-sensitive linker is a carbonate, hydrazone or silyl ether, such as:

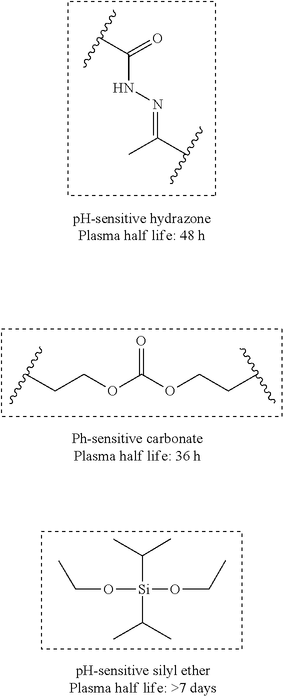

Mammal: This term includes both human and non-human mammals. Similarly, the term “subject” includes both human and veterinary subjects (such as cats, dogs, cows, and pigs) and rodents (such as mice and rats). In some examples, the mammal has or is at risk of developing type 2 diabetes.

MR-L2: A small molecule activator of PDE4 long isoform variants, such as PDE4D3. The chemical name of MR-L2 is 2-(3-(4-chloro-3-fluorophenyl)-5-ethyl-1H-1,2,4-triazol-1-yl)-N-(3,5-dichlorobenzyl) acetamide. The chemical structure of MR-L2 is shown below (see Omar et al., Proc Natl Acad Sci USA 116 (27): 13320-13329, 2019):

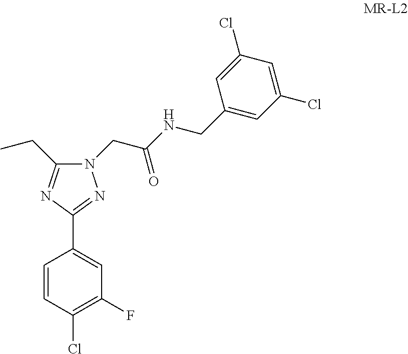 [SMILES: CCc1nc(-c2ccc(Cl)c(F)c2)nn1CC(=O)NCc1cc(Cl)cc(Cl)c1]

Operably linked: A first nucleic acid sequence is operably linked with a second nucleic acid sequence when the first nucleic acid sequence is placed in a functional relationship with the second nucleic acid sequence. For instance, a promoter is operably linked to a coding sequence if the promoter affects the transcription or expression of the coding sequence (such as a PDE4D3 coding sequence). Generally, operably linked DNA sequences are contiguous and, where necessary, join two protein coding regions, in the same reading frame.

Pharmaceutically acceptable carriers: The pharmaceutically acceptable carriers of use are conventional. Remington: The Science and Practice of Pharmacy, 22^{nd} ed., London, UK: Pharmaceutical Press, 2013), describes compositions and formulations suitable for pharmaceutical delivery of the agents described herein. In general, the nature of the carrier will depend on the particular mode of administration being employed. For instance, parenteral formulations usually comprise injectable fluids that include pharmaceutically and physiologically acceptable fluids such as water, physiological saline, balanced salt solutions, aqueous dextrose, glycerol, or the like as a vehicle. For solid compositions (e.g., powder, pill, tablet, or capsule forms), conventional non-toxic solid carriers can include, for example, pharmaceutical grades of mannitol, lactose, starch, or magnesium stearate. In addition to biologically-neutral carriers, pharmaceutical compositions to be administered can contain minor amounts of non-toxic auxiliary substances, such as wetting or emulsifying agents, preservatives, and pH buffering agents and the like, for example sodium acetate or sorbitan monolaurate.

Phosphodiesterase 4D (PDE4D): One of four mammalian counterparts to the fruit fly ‘dunce’ gene. PDE4D proteins have 3′,5′-cyclic-AMP phosphodiesterase activity and degrade cAMP, which acts as a signal transduction molecule in multiple cell types. The PDE4D gene uses different promoters to generate multiple alternatively spliced transcript variants that encode functional proteins (e.g., PDE4D1, PDE4D2, PDE4D3, PDE4D4, PDE4D5, PDE4D6, PDE4D7, PDE4D8 and PDE4D9; see NCBI Gene ID 5144). Nucleic acid and protein sequences for multiple PDE4D isoforms are publicly available, such as under NCBI Gene ID 5144. Exemplary PDE4D3 isoform sequences include GenBank Accession Nos. NM_006203.5 (nucleotide; SEQ ID NO: 1) and NP_006194.2 (protein; SEQ ID NO: 2). In some examples, a PDE4D3 nucleic acid sequence has at least 80%, at least 90%, at least 95%, at least 98%, at least 99% or 100% sequence identity to SEQ ID NO: 1, and encodes a protein having PDE4D3 biological activity. In some examples, a PDE4D3 protein has at least 80%, at least 90%, at least 95%, at least 98%, at least 99% or 100% sequence identity to SEQ ID NO: 2, and has PDE4D3 biological activity. In some example, such a PDE4D3 does not include a substitution at position 44, such as an S44A substitution.

Preventing, treating or ameliorating a disease: “Preventing” a disease refers to inhibiting the full development of a disease. “Treating” refers to a therapeutic intervention that ameliorates a sign or symptom of a disease or pathological condition (such as T2D) after it has begun to develop (such as lowering blood glucose). “Ameliorating” refers to the reduction in the number or severity of signs or symptoms of a disease.

Promoter: An array of nucleic acid control sequences which direct transcription of a nucleic acid. A promoter includes necessary nucleic acid sequences near the start site of transcription, such as, in the case of a polymerase II type promoter, a TATA element. A promoter also optionally includes distal enhancer or repressor elements which can be located as much as several thousand base pairs from the start site of transcription. In some aspects, the promoter is an adipose/adipocyte-specific promoter, such as the adiponectin promoter/enhancer (see, e.g., Wang et al., Endocrinology 151 (6): 2933-2939, 2010). In specific examples, the promoter is the adiponectin promoter/enhancer of SEQ ID NO: 27.

Sequence identity: The similarity between amino acid or nucleotide sequences is expressed in terms of the similarity between the sequences, otherwise referred to as sequence identity. Sequence identity is frequently measured in terms of percentage identity (or similarity or homology); the higher the percentage, the more similar the two sequences. Homologs or variants of a polypeptide will possess a relatively high degree of sequence identity when aligned using standard methods.

Methods of alignment of sequences for comparison are well known in the art. Various programs and alignment algorithms are described in: Smith and Waterman, Adv. Appl. Math. 2:482, 1981; Needleman and Wunsch, J. Mol. Biol. 48:443, 1970; Pearson and Lipman, Proc. Natl. Acad. Sci. U.S.A. 85:2444, 1988; Higgins and Sharp, Gene 73:237, 1988; Higgins and Sharp, CABIOS 5:151, 1989; Corpet et al., Nucleic Acids Research 16:10881, 1988; and Pearson and Lipman, Proc. Natl. Acad. Sci. U.S.A. 85:2444, 1988. Altschul et al., Nature Genet. 6:119, 1994, presents a detailed consideration of sequence alignment methods and homology calculations.

The NCBI Basic Local Alignment Search Tool (BLAST) (Altschul et al., J. Mol. Biol. 215:403, 1990) is available from several sources, including the National Center for Biotechnology Information (NCBI, Bethesda, MD) and on the internet, for use in connection with the sequence analysis programs blastp, blastn, blastx, tblastn, and tblastx. A description of how to determine sequence identity using this program is available on the NCBI website on the internet.

Subject: Any mammal, such as humans, non-human primates, pigs, sheep, cows, dogs, cats, rodents and the like which is to be the recipient of a particular treatment, such as treatment with an agent that increases expression or activity of PDE4D3, as described herein. In some examples, the subject is a human, for example a human subject with or at risk of T2D.

Vector: A nucleic acid molecule as introduced into a host cell, thereby producing a transformed host cell. A vector may include nucleic acid sequences that permit it to replicate in the host cell, such as an origin of replication. A vector may also include one or more PDE4D3 coding sequences, and/or selectable marker genes (such as antibiotic resistance or an enzyme) and other genetic elements. A vector can transduce, transform, or infect a cell, thereby causing the cell to express nucleic acids and/or proteins other than those native to the cell. A vector optionally includes materials to aid in achieving entry of the nucleic acid into the cell, such as a viral particle, liposome, protein coating, or the like. In some aspects, the vector is an adeno-associated virus (AAV) vector, an adenovirus (Ad) vector, or a lentivirus vector. In some aspects, the vector is a plasmid.

### III. Introduction

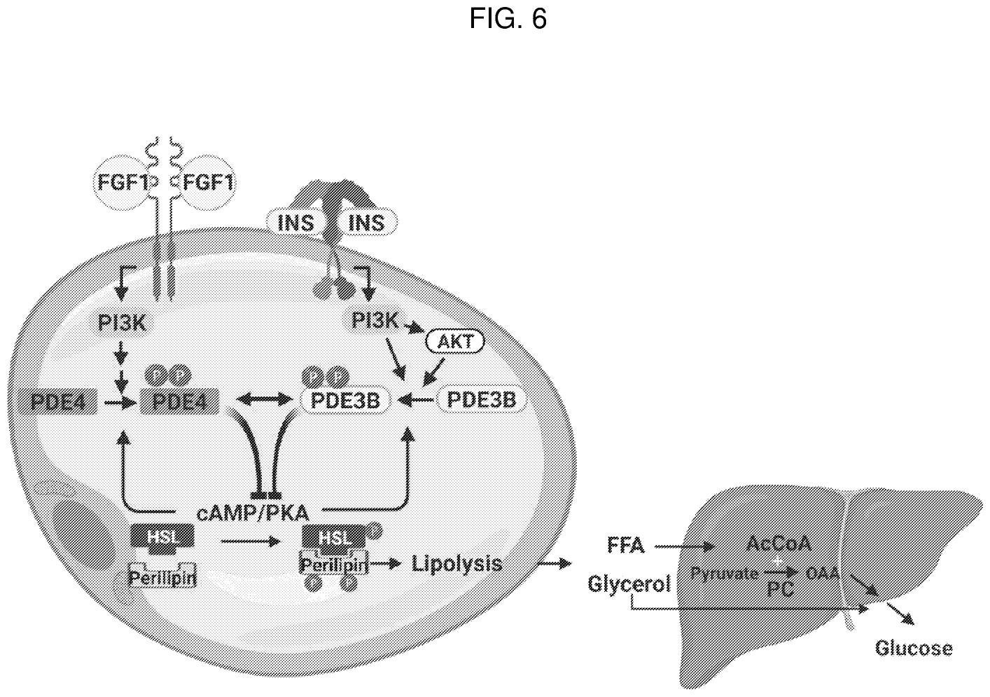

Adipose tissue holds 80-85% of the body's energy reserves, hence the decision to store or release is central to physiological homeostasis. However, while adipose lipolysis is triggered by multiple factors, insulin is the only known anti-lipolytic regulator. Disclosed herein is a signaling cascade induced by FGF1 that functions as an alternate lipolytic suppressive pathway and establishes FGF1 as a regulator of fatty acid homeostasis. FGF1 is an essential mediator of adipose remodeling (Jonker et al., 2012) and rapidly normalizes blood glucose levels in diabetic mouse models in an adipose FGFR1-dependent manner (Suh et al., 2014). The present disclosure demonstrates that FGF1-FGFR1 signaling reduces blood glucose levels by suppressing adipose lipolysis and thereby, decreases HGP through the allosteric regulation of pyruvate carboxylase. Within the adipocyte, FGF1 engages an alternate PI3K/PDE4D circuit to inhibit the cAMP-PKA axis. The resultant reductions in cAMP decrease PKA-mediated phosphorylation of HSL and its subsequent translocation to the lipid droplet. The PDE4D dependency of the anti-lipolytic and anti-diabetic actions of FGF1 suggest a parallel pathway to the established insulin-PDE3B axis (FIG. 6). While both insulin and PKA activate PDE3B via phosphorylation (Degerman et al., 2011), the results disclosed herein reveal a parallel relationship between FGF1 and PDE4D in which phosphorylation of PDE4D3-S44 engages a negative feed-back loop to lower cAMP levels (Mackenzie et al., 2002; Oki et al., 2000; Sette and Conti, 1996). Convergence of both anti-lipolytic (FGF1) and lipolytic (cAMP/PKA) stimuli on PDE4D phosphorylation indicates a functional compartmentalization of this pathway in different membrane regions as observed with PDE3B regulation (Ahmad et al., 2009).

Mutation of S85 (QRRES motif, SEQ ID NO: 36), which enhances in vitro PDE4D3 activity (Sette and Conti, 1996), had a minor effect on suppression of lipolysis when PDE4D3-S85A was overexpressed in adipocytes. In contrast, mutation of S44 (FRRHS motif; SEQ ID NO: 37) abrogated the suppression of lipolysis by PDE4D3 despite overexpression, indicating a novel regulatory role for this phosphorylation site. In support of this, over-expression of WT and S44A PDE4D3 resulted in similar PDE activities in adipocytes. The N-terminus of PDE4D3 and its PKA phosphorylation site are implicated in the interaction of PDE4D3 with the muscle-specific A-kinase anchoring protein (mAKAP) (Carlisle Michel et al., 2004; Dodge et al., 2001). Hence, this site may be pivotal in compartmentalization of PKA-PDE4D3 complexes. In addition, binding of phosphatidic acid to the N-terminal sequence activates PDE4 (Grange et al., 2000).

While the present disclosure describes the action of FGF1 in adipose tissue, central administration of a single FGF1 dose can also restore glucose homeostasis in diabetic models, however the kinetics are slow (weeks). The improvement in blood glucose levels in the Zucker T2D diabetic rat model was attributed to preservation of β cell function and increased hepatic glucose uptake, whereas a suppression of the hypothalamic-pituitary-adrenal (HPA) axis resulting in lower lipolysis and hepatic glucose output was described in T1D rats (Perry et al., 2015b; Scarlett et al., 2019; Scarlett et al., 2016). While the rapid glucose lowering seen with peripheral FGF1 delivery is consistent with lipolytic regulation, the delayed effects of central FGF1 delivery suggest a distinct mechanism.

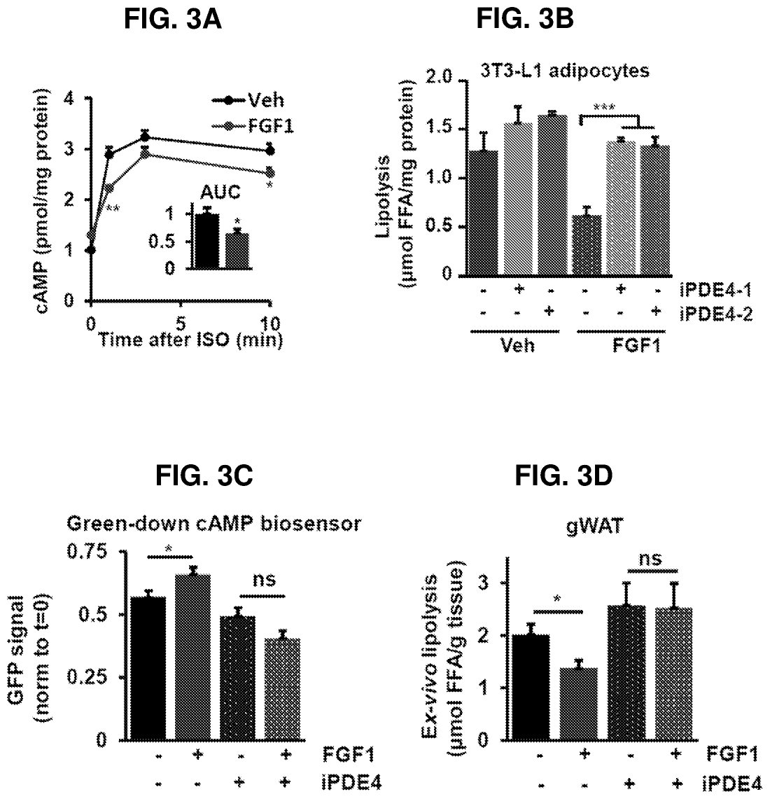

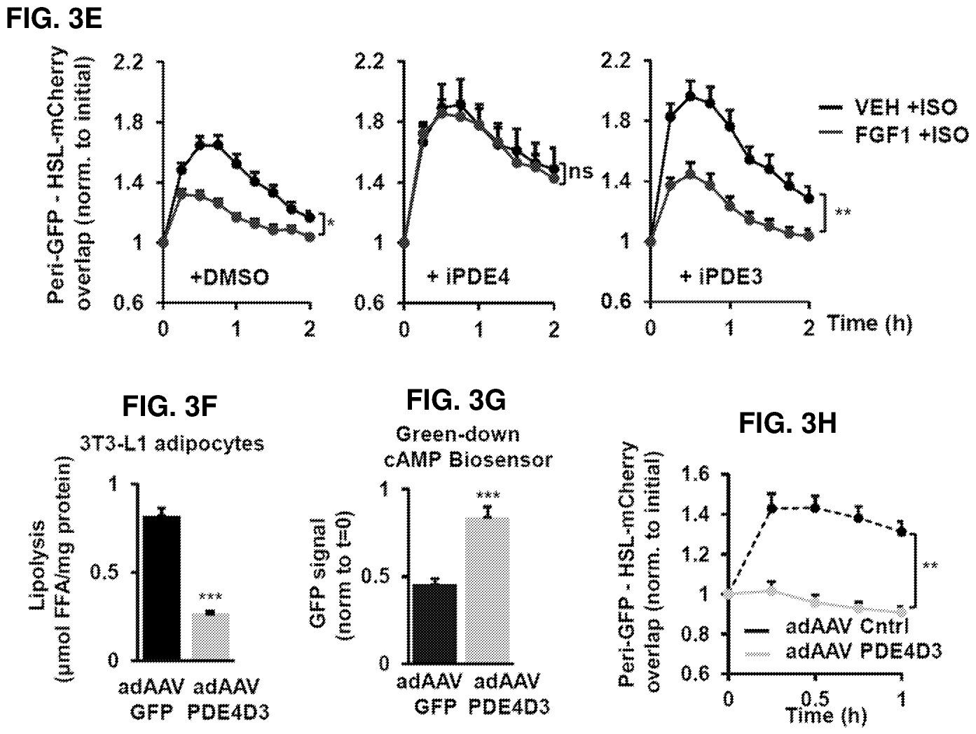

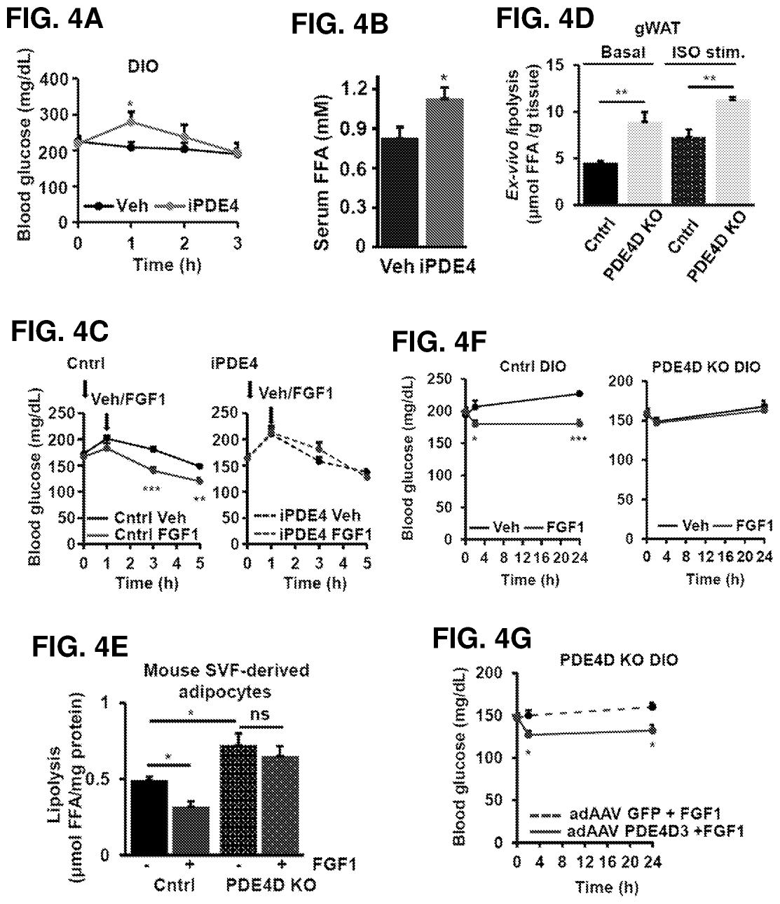

The findings disclosed herein that implicate adipose PDE4D in the beneficial actions of FGF1 appear at odds with the anti-diabetic effects of systemic PDE4 inhibition (Möllmann et al., 2017; Vollert et al., 2012). Increased GLP1 secretion, higher serum insulin levels, and increased muscle mitochondrial function are associated with chronic PDE4 inhibition indicating adipose tissue independent effects. (Ong et al., 2009; Park et al., 2012; Vollert et al., 2012). In addition, contributions of the associated weight loss and potential anti-inflammatory effects of PDE4B inhibition to improvements in glucose tolerance and fasting glucose levels are not known (Jin and Conti, 2002; Komatsu et al., 2013; Möllmann et al., 2017; Zhang et al., 2009). In contrast to the pleiotropic effects of systemic inhibition, the present study indicates that PDE4D activity in adipocytes is necessary for FGF1 to suppress lipolysis and lower blood glucose levels (FIG. 3 and FIG. 4).

PDE3B is important for controlling cAMP levels and lipolysis in adipocytes, however PDE4 accounts for approximately half of the total PDE activity in white adipocytes (Young et al., 2006). Moreover, maximal adipose lipolysis requires both PDE3 and PDE4 inhibitors, supporting an underappreciated role of PDE4 in the regulation of lipolysis (DiPilato et al., 2015b; Kraynik et al., 2013; Snyder et al., 2005). In addition, the finding that FGF1 enhances PDE4D3 phosphorylation at S44 correlates with the post-prandial increases seen at this site in both chow and HFD mice (FIG. 5M). The data agree with previous work which showed the anti-lipolytic potential of PDE4 is higher in the fed-state suggesting a potential regulation of PDE4 activity/levels via feeding-fasting cycles (Nakamura et al., 2004). The data disclosed herein indicate the involvement of adipose PDE4D in physiological control of lipolysis in vivo. Furthermore, the enhanced lipolysis observed in PDE4D KO mice support this conclusion. Given that PDE3B levels and activity are decreased in diabetic mouse models, the FGF1/PDE4 regulatory pathway may be increasingly relevant in metabolically stressed states (Tang et al., 2001).

The anti-lipolytic effect of FGF1 in the fed state contrasts to FGF21-induced lipolysis in the fasted state. Accordingly, these findings implicate an unexpected FGF1: FGF21 molecular balance regulating the storage and release of fat in the fed and fasted state, respectively. This physiologic paradigm describes a mechanism that not only manages glucose homeostasis in health, but via FGF1 injection, can be used to quickly rebalance glucose levels in insulin resistant type 2 diabetes. Thus, in addition to a new signaling cascade that suppresses lipolysis, these findings reveal the therapeutic potential of the FGF1-PDE4D axis in diabetes.

### IV. Methods of Lowering Blood Glucose and Treating Type 2 Diabetes

Described herein is the finding that fibroblast growth factor 1 (FGF1) suppresses adipose lipolysis and the anti-lipolytic activities of FGF1 are required for acute glucose lowering. Also described is the finding that these FGF1 activities are mediated by the activation of phosphodiesterase 4D isoform 3 (PDE4D3) in adipose tissue. In view of these findings, the present disclosure provides methods of lowering blood glucose and/or treating T2D in a subject by increasing expression or activity of PDE4D3. In some examples, blood glucose is lowered by at least 10%, at least 15%, at least 20%, at least 25%, at least 50%, or at least 75%, for example relative to an amount prior to treatment or an amount without use of the disclosed methods. In some examples, phosphodiesterase 4D isoform 3 (PDE4D3) activity and/or expression is increased using the disclosed methods by at least 10%, at least 20%, at least 25%, at least 50%, at least 75%, at least 90%, at least 100%, at least 200%, or at least 500%, for example relative to an amount prior to treatment with the agent that increases expression or activity of PDE4D3 or an amount without use of the agent that increases expression or activity of PDE4D3.

Provided herein is a method of reducing blood glucose in a subject. In some aspects, the method includes administering to the subject a therapeutically effective amount of an agent that increases expression or activity of PDE4D3 in adipocytes of the subject. In some examples, the subject has been diagnosed with T2D, or is at high risk of developing T2D.

Also provided herein is a method of treating T2D in a subject. In some aspects, the method includes administering to the subject a therapeutically effective amount of an agent that increases expression or activity of PDE4D3 in adipocytes of the subject.

Also provided are agents that can be used in such methods, such as a nucleic acid molecule encoding a PDE4D3 protein operably linked to an adipocyte-specific promoter, and vectors (such as plasmids or viral vectors) that include such. In one example the agent is a small molecule activator of PDE4D3 conjugated to an antibody that specifically binds fibroblast growth factor receptor 1b (FGFR1b), such as an antibody disclosed in US 2012/0121609. In one example the agent is a small molecule activator of PDE4D3 conjugated to an incretin, such as GIP or GLP-1. Compositions that include such agents are also provided, for example which may further include a pharmaceutically acceptable carrier such as water, saline, or a buffer.

In some aspects, the agent that increases expression or activity of PDE4D3 includes a nucleic acid molecule (e.g., DNA, cDNA, or RNA) encoding a PDE4D3 protein. In some examples, the nucleic acid molecule encoding the PDE4D3 protein is operably linked to an adipocyte-specific promoter and/or enhancer, such as the adiponectin promoter and/or enhancer. In particular non-limiting examples, the adiponectin promoter/enhancer includes SEQ ID NO: 27. In some examples, the nucleic acid molecule further includes a target sequence for miR-122a, such as SEQ ID NO: 28, to restrict expression to adipose tissue.

In some aspects, the nucleic acid molecule encoding the PDE4D3 protein includes a vector. In some examples, the vector is a viral vector, such as but not limited to, an adenovirus vector, an adeno-associated virus (AAV) vector, or a lentivirus vector. In other examples, the vector is a plasmid vector.

In other aspects, the nucleic acid molecule encoding the PDE4D3 protein is introduced into adipocytes using a gene editing method. Gene editing methods are known and include, for example, CRISPR-Cas9, Cas13d, TALENs, or ZFNs. Gene editing techniques are known, such as those described in U.S. Pat. No. 10,000,772; and U.S. Publication Nos. 2021/0340568, 2021/0338815, 2021/0292769, 2021/0222171.

In some aspects of the disclosed methods, the PDE4D3 protein includes the amino acid sequence set forth as SEQ ID NO: 1, or a variant thereof having at least 85%, at least 90%, at least 95%, at least 96%, at least 97%, at least 98% or at least 99%, or 100% identity to SEQ ID NO: 1. In some examples, such a protein includes a native Ser at position 44. In some examples, such a protein does not include a S44A substitution.

In some aspects, the agent that increases expression or activity of PDE4D is a small molecule activator of PDE4D3. In some examples, the small molecule activator of PDE4D3 is an N-substituted-2-(3-aryl-1H-1,2,4-triazol-1-yl) acetamide. In specific non-limiting examples, the small molecule activator of PDE4D3 is 2-(3-(4-chloro-3-fluorophenyl)-5-ethyl-1H-1,2,4-triazol-1-yl)-N-(3,5-dichlorobenzyl) acetamide (also known as MR-L2).

In some examples, the small molecule activator of PDE4D3 is conjugated to an antibody that specifically binds fibroblast growth factor receptor 1b (FGFR1b), such as a polyclonal FGFR1b antibody, a monoclonal FGFR1b antibody, or fragment of an FGFR1b antibody (such as an antibody provided in US 2012/0121609). In other examples, the small molecule activator of PDE4D3 is conjugated to an incretin, such as but not limited to, gastric inhibitory peptide (GIP) or glucagon-like peptide-1 (GLP-1), such as a peptide having at least 80%, at least 85%, at least 90%, at least 95%, at least 96%, at least 97%, at least 98% or at least 99%, or 100% identity to SEQ ID NO: 24 or 26. In particular examples, the small molecule activator of PDE4D3 is conjugated to the antibody or the incretin via an acid-sensitive linker.

The agent that increases expression or activity of PDE4D3, or a pharmaceutical composition thereof, can be administered to a subject, such as a mammalian subject (e.g., a human subject), by any means, including orally, intravenously, intramuscularly, intraperitoneally, intranasally, intradermally, intrathecally, subcutaneously, via inhalation or via suppository. In one non-limiting example, the composition is administered via injection. In some examples, site-specific administration of the composition can be used, for example by administering the agent to adipose tissue (for example by using a pump, or by implantation of a slow release form at the adipose depot). The particular mode of administration and the dosage regimen will be selected by the attending clinician, taking into account the particulars of the case (e.g., the subject, the disease, the disease state involved, the particular treatment, and whether the treatment is prophylactic). Treatment can involve daily or multi-daily or less than daily (such as weekly, every other week, monthly, every 7 days, every 10 days, every 14 days, every 21 days, every 30 days, every 40 days, every 60 days, etc.) doses of the agent or pharmaceutical composition over a period of a few days, few weeks, to months, or even years. For example, a therapeutically effective amount of the agent can be administered in a single dose, once daily, twice daily, three times daily, four times daily, six times daily, weekly, every other week, every three weeks, every month, every other month, or in several doses, for example daily, or during a course of treatment. In a particular non-limiting example, treatment involves once daily dose, twice daily dose, once weekly dose, every other week dose, or monthly dose.

When a viral vector is utilized for administration of a nucleic acid encoding a PDE4D3 protein (such as the PDE4D3 protein of SEQ ID NO: 2, or a protein having at least 80%, at least 85%, at least 90%, at least 95%, at least 96%, at least 97%, at least 98%, at least 99%, or 100% sequence identity to SEQ ID NO: 2), the recipient can receive a dose in the range of from about 105 to about 1010 plaque forming units/mg, although a lower or higher dose can be administered. Examples of methods for administering the composition to a subject (such as a mammalian subject) include, but are not limited to, exposure of cells to the recombinant virus ex vivo, or injection of the composition into the affected tissue or intravenous, subcutaneous, intradermal, or intramuscular administration of the virus. Alternatively, the viral vector may be administered locally by direct injection into adipose tissue in a pharmaceutically acceptable carrier. Generally, the quantity of viral vector carrying the PDE4D3 nucleic acid sequence to be administered (such as SEQ ID NO: 1, or a nucleic acid sequence having at least 80%, at least 85%, at least 90%, at least 95%, at least 96%, at least 97%, at least 98%, at least 99%, or 100% sequence identity to SEQ ID NO: 1), is based on the titer of virus particles. An exemplary range to be administered is 105 to 1010 virus particles.

In some aspects, the methods further include administering to the subject a therapeutically effective amount of a mature FGF1 protein or a modified mature FGF1 protein. In some examples, the modified mature FGF1 protein has reduced mitogenicity and/or increased stability compared to native FGF1 protein, and/or the modified mature FGF1 protein selectively binds FGFR1b. Exemplary FGF1 proteins (including mutant FGF1 proteins) are described in, for example, WO 2015/061331, WO 2015/061351, WO 2015/061361, WO 2011/130729, WO 2016/172153, WO 2016/172156, WO 2016/172290, WO 2018/026713, WO 2018/112200 and US 2021/0032303. In specific examples, the mutant FGF1 protein is one shown in FIG. 12 (SL001, SL002, SL003 or SL004, set forth herein as SEQ ID NOs: 29-32, respectively). The amount of FGF1 protein (such as mutated FGF1 protein) administered can be dependent on the subject being treated, the severity of the affliction, and the manner of administration, and can be left to the judgment of the prescribing clinician. The formulation to be administered contains a quantity of the FGF1 protein in amounts effective to achieve the desired effect in the subject being treated. In specific examples, a therapeutically effective amount of a mutated FGF1 protein can be the amount of the protein (or a nucleic acid encoding the FGF1 protein) that is necessary to treat diabetes or reduce blood glucose levels (for example a reduction of at least 5%, at least 10% or at least 20%, for example relative to no administration of the mutant FGF1). In some examples, mature FGF1 or modified mature FGF1 protein is administered in combination with a therapeutically effective amount of another mutant FGF1, such as one shown in FIG. 12 or provided in any of U.S. Pat. Nos. 8,906,854; 8,999,929; 9,925,241, and 9,925,243; US Patent Application Publication Nos. US-2016-0237133-A1; US-2017-0355739-A1; US-2018-0057554-A1; US-2018-0228869-A1; US 2018-0050087 A1; and US-2019-0151416-A1, and PCT Publication WO 2018/112200.

In some aspects, the method further includes administering a therapeutically effective amount of an additional therapeutic agent, such as an agent useful in the treatment of diabetes. Anti-diabetic agents are generally categorized into six classes: biguanides (e.g., metformin); thiazolidinediones (including rosiglitazone (Avandia®), pioglitazone (Actos®), rivoglitazone, and troglitazone); sulfonylureas; inhibitors of carbohydrate absorption; fatty acid oxidase inhibitors and anti-lipolytic drugs; and weight-loss agents. Any of these agents can be used in the methods disclosed herein. In some examples, the additional therapeutic agent is insulin, an alpha-glucosidase inhibitor, amylin agonist, dipeptidyl-peptidase 4 (DPP-4) inhibitor, meglitinide, sulfonylurea, or a peroxisome proliferator-activated receptor (PPAR)-gamma agonist. In particular examples, the PPAR-gamma agonist is a thiazolidinedione (such as pioglitazone, rosiglitazone, rivoglitazone, or troglitazone), aleglitazar, farglitazar, muraglitazar, or tesaglitazar.

In some examples, an agent that increases expression or activity of PDE4D3 is administered in combination with effective doses of one or more FGF1 proteins (such as mutant FGF1 proteins, e.g., a mutant shown in FIG. 12, set forth as SEQ ID NOs: 29-32), anti-diabetic agents (such as biguanides, thiazolidinediones, or incretins) and/or lipid lowering compounds (such as statins or fibrates). The terms “administration in combination,” “co-administration,” or the like, refer to both concurrent (e.g., contemporaneous) and sequential administration of the active agents. Administration of an agent that increases expression or activity of PDE4D3 may also be in combination with lifestyle modifications, such as increased physical activity, low fat diet, low sugar diet, and smoking cessation.

In some aspects, the subject is a mammal. In some examples, the subject is a human, dog or cat, such as a human, dog or cat with elevated blood glucose. In particular non-limiting examples, the subject is a human who has or is at risk of T2D.

In some aspects, the method further includes selecting a subject with elevated blood glucose, selecting a subject who is at risk of T2D, selecting a subject who has pre-diabetes, or selecting a subject who has T2D.

In some examples, a subject with diabetes may be clinically diagnosed by a fasting plasma glucose (FPG) concentration of greater than or equal to 7.0 millimole per liter (mmol/L) (126 milligram per deciliter (mg/dL)), or a plasma glucose concentration of greater than or equal to 11.1 mmol/L (200 mg/dL) at about two hours after an oral glucose tolerance test (OGTT) with a 75 gram (g) load, or in a patient with classic symptoms of hyperglycemia or hyperglycemic crisis, a random plasma glucose concentration of greater than or equal to 11.1 mmol/L (200 mg/dL), or HbA1c levels of greater than or equal to 6.5%. In other examples, a subject with pre-diabetes may be diagnosed by impaired glucose tolerance (IGT). An OGTT two-hour plasma glucose of greater than or equal to 140 mg/dL and less than 200 mg/dL (7.8-11.0 mM), or a fasting plasma glucose (FPG) concentration of greater than or equal to 100 mg/dL and less than 125 mg/dl (5.6-6.9 mmol/L), or HbA1c levels of greater than or equal to 5.7% and less than 6.4% (5.7-6.4%) is considered to be IGT, and indicates that a subject has pre-diabetes. Additional information can be found in Standards of Medical Care in Diabetes—2010 (American Diabetes Association, Diabetes Care 33: S11-61, 2010).

In some examples, treating T2D includes one or more of increasing glucose tolerance (such as an increase of at least 5%, at least 10%, at least 20%, or at least 50%, for example relative to no administration of the agent), decreasing insulin resistance (for example, decreasing plasma glucose levels, decreasing plasma insulin levels, or a combination thereof, such as decreases of at least 5%, at least 10%, at least 20%, or at least 50%, for example relative to no administration of the agent), decreasing serum triglycerides (such as a decrease of at least 10%, at least 20%, or at least 50%, for example relative to no administration of the agent), decreasing free fatty acid levels (such as a decrease of at least 5%, at least 10%, at least 20%, or at least 50%, for example relative to no administration of the agent), and decreasing HbA1c levels in the subject (such as a decrease of at least 0.5%, at least 1%, at least 1.5%, at least 2%, or at least 5% for example relative to no administration of the agent). In some aspects, the disclosed methods include measuring glucose tolerance, insulin resistance, plasma glucose levels, plasma insulin levels, serum triglycerides, free fatty acids, and/or HbA1c levels in a subject.

### V. Nucleic Acid Molecules and Vectors Encoding PDE4D3

In some aspects of the methods disclosed herein, a nucleic acid molecule encoding a PDE4D3 protein is administered to a subject. Based on the genetic code, nucleic acid sequences coding for PDE4D3 protein can be routinely generated. In some examples, such a sequence is optimized for expression in particular host cells, such as mammalian host cells (e.g., human cells). Thus, in some aspects, the agents disclosed herein include a nucleic acid molecule encoding a PDE4D3 protein.

In some aspects, the amino acid sequence of the PDE4D3 protein is at least 80%, at least 85%, at least 90%, at least 95%, at least 96%, at least 97%, at least 98%, at least 99% or 100% identical to the amino acid sequence of SEQ ID NO: 2. A nucleic acid sequence that codes for a PDE4D3 protein having at least 80%, at least 85%, at least 90%, at least 95%, at least 96%, at least 97%, at least 98%, at least 99% or 100% sequence identity to SEQ ID NO: 2 can readily be produced using the PDE4D3 amino acid sequence provided herein (or publicly available PDE4D3 protein sequences), and the genetic code. In addition, a variety of clones containing functionally equivalent nucleic acids, such as nucleic acids which differ in sequence but which encode the same PDE4D3 protein sequence, can be generated. In some examples, the nucleic acid molecule includes SEQ ID NO: 1, or a variant thereof having at least 80%, at least 85%, at least 90%, at least 95%, at least 96%, at least 97%, at least 98%, at least 99% or 100% sequence identity to SEQ ID NO: 1.

Nucleic acid molecules include DNA, cDNA, and RNA sequences which encode a PDE4D3 protein. Silent mutations in the coding sequence result from the degeneracy (i.e., redundancy) of the genetic code, whereby more than one codon can encode the same amino acid residue. Thus, for example, leucine can be encoded by CTT, CTC, CTA, CTG, TTA, or TTG; serine can be encoded by TCT, TCC, TCA, TCG, AGT, or AGC; asparagine can be encoded by AAT or AAC; aspartic acid can be encoded by GAT or GAC; cysteine can be encoded by TGT or TGC; alanine can be encoded by GCT, GCC, GCA, or GCG; glutamine can be encoded by CAA or CAG; tyrosine can be encoded by TAT or TAC; and isoleucine can be encoded by ATT, ATC, or ATA. Tables showing the standard genetic code can be found in various sources (see, for example, Stryer, 1988, Biochemistry, 3^{rd} Edition, W.H. 5 Freeman and Co., NY).

Codon preferences and codon usage tables for a particular species can be used to engineer isolated nucleic acid molecules encoding a PDE4D3 protein (such as one encoding a protein having at least 80%, at least 85%, at least 90%, at least 95%, at least 96%, at least 97%, at least 98%, at least 99%, or 100% sequence identity to SEQ ID NO: 2) that take advantage of the codon usage preferences of that particular species. For example, the proteins disclosed herein can be designed to have codons that are preferentially used by a particular organism of interest, such as humans or other mammals.

In some examples, the nucleic acid encodes a PDE4D3 protein having a native Ser at position 44. In some examples, such a PDE4D3 protein does not include a S44A substitution.

A nucleic acid encoding the desired protein can be cloned or amplified by in vitro methods, such as the polymerase chain reaction (PCR), the ligase chain reaction (LCR), the transcription-based amplification system (TAS), the self-sustained sequence replication system (3SR) and the Qβ replicase amplification system (QB). A variety of cloning and in vitro amplification methodologies can be used. In addition, nucleic acids encoding sequences encoding a desired protein can be prepared by cloning techniques. Examples of appropriate cloning and sequencing techniques, and instructions sufficient to direct persons of skill through cloning are found in Sambrook et al. (ed.), Molecular Cloning: A Laboratory Manual 2nd ed., vol. 1-3, Cold Spring Harbor Laboratory Press, Cold Spring, Harbor, N.Y., 1989, and Ausubel et al., (1987) in “Current Protocols in Molecular Biology,” John Wiley and Sons, New York, N.Y.

Nucleic acid sequences encoding a PDE4D3 protein can be prepared by any suitable method including, for example, cloning of appropriate sequences or by direct chemical synthesis by methods such as the phosphotriester method of Narang et al., Meth. Enzymol. 68:90-99, 1979; the phosphodiester method of Brown et al., Meth. Enzymol. 68:109-151, 1979; the diethylphosphoramidite method of Beaucage et al., Tetra. Lett. 22:1859-1862, 1981; the solid phase phosphoramidite triester method described by Beaucage & Caruthers, Tetra. Letts. 22 (20): 1859-1862, 1981, for example, using an automated synthesizer as described in, for example, Needham-VanDevanter et al., Nucl. Acids Res. 12:6159-6168, 1984; and, the solid support method of U.S. Pat. No. 4,458,066. Chemical synthesis produces a single stranded oligonucleotide. This can be converted into double stranded DNA by hybridization with a complementary sequence, or by polymerization with a DNA polymerase using the single strand as a template. One of skill would recognize that while chemical synthesis of DNA is generally limited to sequences of about 100 bases, longer sequences may be obtained by the ligation of shorter sequences.

In some aspects herein, the PDE4D3 nucleic acid coding sequence (such as a sequence encoding SEQ ID NO: 2, or encoding a protein having at least 80%, at least 85%, at least 90%, at least 95%, at least 96%, at least 97%, at least 98%, at least 99%, or 100% sequence identity to SEQ ID NO: 2, which in some examples does not have an S44A substitution) can be inserted into an expression vector including, but not limited to a plasmid, virus or other vehicle that can be manipulated to allow insertion or incorporation of sequences and can be expressed in, for example, a mammalian (such as human) host.

Nucleic acid sequences encoding a PDE4D3 protein (such as a nucleic acid encoding SEQ ID NO: 2, or encoding a protein having at least 80%, at least 85%, at least 90%, at least 95%, at least 96%, at least 97%, at least 98%, at least 99%, or 100% sequence identity to SEQ ID NO: 2) can be operatively linked to expression control sequences. An expression control sequence operatively linked to a desired protein coding sequence is ligated such that expression of the coding sequence is achieved under conditions compatible with the expression control sequences. The expression control sequences include, but are not limited to appropriate promoters, enhancers, transcription terminators, a start codon (i.e., ATG) in front of a PDE4D3 protein-encoding gene, splicing signal for introns, maintenance of the correct reading frame of that gene to permit proper translation of mRNA, and stop codons. In some examples, the nucleic acid sequence is operably linked to a promoter and/or enhancer that allows for specific expression in adipocytes and/or adipose tissue. In specific examples, the adipocyte-specific promoter/enhancer is an adiponectin promoter/enhancer. In other specific examples, the nucleic acid molecule includes (or further includes) a target sequence for an adipose-specific microRNA, such as miR-122a. In specific instances, the miR-122a target sequences includes or consists of SEQ ID NO: 28.

In some aspects, the nucleic acid molecule is a viral vector that encodes a PDE4D3 protein (such as SEQ ID NO: 2, or a protein having at least 80%, at least 85%, at least 90%, at least 95%, at least 96%, at least 97%, at least 98%, at least 99%, or 100% sequence identity to SEQ ID NO: 2). Exemplary viral vectors include adenovirus, adeno-associated virus (AAV), polyoma, SV40, vaccinia virus, herpes viruses (including HSV and EBV), Sindbis viruses, alphaviruses and retroviruses of avian, murine, and human origin (e.g., lentivirus vectors). Other suitable vectors include orthopox vectors, avipox vectors, fowlpox vectors, capripox vectors, suipox vectors, and poliovirus vectors. In particular examples, the viral vector is an adenovirus vector, an AAV vector or a lentivirus vector.

Viral vectors that encode a PDE4D3 protein (such as SEQ ID NO: 2, or encode a protein having at least 80%, at least 85%, at least 90%, at least 95%, at least 96%, at least 97%, at least 98%, at least 99%, or 100% sequence identity to SEQ ID NO: 2) can include at least one expression control element operationally linked to the nucleic acid sequence encoding the protein. The expression control elements are inserted in the vector to control and regulate the expression of the nucleic acid sequence. Examples of expression control elements of use in these vectors includes, but is not limited to, lac system, operator and promoter regions of phage lambda, yeast promoters and promoters derived from polyoma, adenovirus, retrovirus or SV40. In some examples, the vector includes a promoter and/or enhancer that allows for specific expression in adipocytes and/or adipose tissue. In specific examples, the adipocyte-specific promoter/enhancer is an adiponectin promoter/enhancer. In some examples, the vector includes or further includes a target sequence for miR-122a, such as SEQ ID NO: 28.

Additional operational elements for a viral vector include, but are not limited to, leader sequence, termination codons, polyadenylation signals and any other sequences necessary for the appropriate transcription and subsequent translation of the nucleic acid sequence encoding the protein in the host. The expression vector can contain additional elements necessary for the transfer and subsequent replication of the expression vector containing the nucleic acid sequence in a host system. Examples of such elements include, but are not limited to, origins of replication and selectable markers. It will further be understood by one skilled in the art that such vectors are easily constructed using conventional methods (Ausubel et al., (1987) in “Current Protocols in Molecular Biology,” John Wiley and Sons, New York, N.Y.) and are commercially available.

### VI. Activators of PDE4D3

In some aspects of the disclosed methods, the agent that increases expression or activity of PDE4D3 is a small molecule activator of PDE4D3. In some examples, the small molecule activator of PDE4D3 is an N-substituted-2-(3-aryl-1H-1,2,4-triazol-1-yl) acetamide. In particular non-limiting examples, the N-substituted-2-(3-aryl-1H-1,2,4-triazol-1-yl) acetamide is 2-(3-(4-chloro-3-fluorophenyl)-5-ethyl-1H-1,2,4-triazol-1-yl)-N-(3,5-dichlorobenzyl) acetamide (MR-L2). As described in Omar et al. (Proc Natl Acad Sci USA 116 (27): 13320-13329, 2019), MR-L2 is an allosteric activator of PDE4 long form cyclic AMP phosphodiesterases (such as PDE4D3).

In some aspects, the small molecule activator of PDE4D3 (such as MR-L2) is conjugated to a molecule that directs delivery of the small molecule to adipose tissue/adipocytes. Thus, in some aspects, the agents disclosed herein include a small molecule activator of PDE4D3, for example conjugated to a molecule that directs delivery of the small molecule to adipose tissue/adipocytes. In some examples, the small molecule is conjugated to an antibody or fragment thereof (such as an Fab fragment), such as a monoclonal antibody, that specifically binds a protein expressed on adipocytes, such as fibroblast growth factor receptor 1b (FGFR1b). Antibodies that specifically bind FGFR1b are known (see, e.g., U.S. 2012/0121609) and can be generated and/or identified using standard methods, such as by phage display or immunization of mice or rabbits.

In other examples, the small molecule activator of PDE4D3 is conjugated to an incretin, such as GIP or GLP-1. The receptors for GIP (GIPR) and GLP-1 (GLP-1R) are expressed in adipose tissue (Capozzi et al., Endocrine Reviews 39 (5): 719-738, 2018). Thus, conjugation of the small molecule activator to GIP or GLP-1, targets the molecule to adipose tissue.

In some examples, the small molecule activator (such as MR-L2) is conjugated to a monoclonal antibody (such as an FGFR1b-specific antibody) or an incretin (such as GIP or GLP-1) via an acid-sensitive linker. Upon delivery of the conjugated molecules to adipocytes, the conjugates are internalized and the small molecular activator is cleaved from the antibody or incretin within endosomes, which have a pH range of about 6.5 (early endosomes) to about 4.5 (lysosomes). Acid-sensitive linkers are well-known (see, e.g., Zhuo et al., Molecules 25:5649, 202); appropriate acid-sensitive linkers can be selected by a skilled person. In some examples, the acid-sensitive linker is a carbonate, hydrazone or silyl ether linker.

### VII. Pharmaceutical Compositions

In the context of the disclosed methods, an agent that increases expression or activity of PDE4D3 (such as a nucleic acid molecule/vector encoding PDE4D3, or a small molecule activator of PDE4D3) can be administered to the subject as part of a pharmaceutical composition. Such pharmaceutical compositions can be formulated with an appropriate pharmaceutically acceptable carrier, depending upon the particular mode of administration chosen. Thus, in some aspects, the agents disclosed herein are part of a pharmaceutical composition.

In some aspects, the pharmaceutical composition consists essentially of a nucleic acid (or vector) encoding a PDE4D3 protein and a pharmaceutically acceptable carrier. In other aspects, the pharmaceutical composition consists essentially of a small molecule activator of PDE4D3 (such as MR-L2) and a pharmaceutically acceptable carrier. In these aspects, additional therapeutically effective agents are not included in the compositions.

In other aspects of the disclosed methods, the pharmaceutical composition includes one or more additional therapeutic agents, such as agents for the treatment of T2D. Thus, the pharmaceutical compositions can include a therapeutically effective amount of another agent. Examples of such agents include, without limitation, antidiabetic agents for example, insulin, metformin, sulphonylureas (e.g., glibenclamide, tolbutamide, glimepiride), nateglinide, repaglinide, thiazolidinediones (e.g., rosiglitazone, pioglitazone), peroxisome proliferator-activated receptor (PPAR)-γ agonists (such as C1262570, aleglitazar, farglitazar, muraglitazar, tesaglitazar, and TZD) and PPAR-γ antagonists, PPAR-gamma/alpha modulators (such as KRP 297), alpha-glucosidase inhibitors (e.g., acarbose, voglibose), dipeptidyl peptidase (DPP)-IV inhibitors (such as LAF237, MK-431), alpha2-antagonists, agents for lowering blood sugar, cholesterol-absorption inhibitors, 3-hydroxy-3-methylglutaryl-coenzyme A (HMGCoA) reductase inhibitors (such as a statin), insulin and insulin analogues, GLP-1 and GLP-1 analogues (e.g. exendin-4) or amylin. In some examples, the pharmaceutical composition includes, or further includes, a FGF1 protein, or a mutant FGF1 protein, such as one or more FGF1 proteins shown in FIG. 12 or disclosed in WO 2015/061331, WO 2015/061351, WO 2015/061361, WO 2011/130729, WO 2016/172153, WO 2016/172156, WO 2016/172290, WO 2018/026713, WO 2018/112200 or US 2021/0032303. In specific examples, the pharmaceutical composition containing a mutated FGF1 protein can further include a therapeutically effective amount of other FGFs, such as FGF21, FGF19, or both, heparin, or combinations thereof.

The pharmaceutically acceptable carriers and excipients useful in this disclosure are conventional. See, e.g., Remington: The Science and Practice of Pharmacy, The University of the Sciences in Philadelphia, Editor, Lippincott, Williams, & Wilkins, Philadelphia, PA, 21^{st} Edition (2005). For instance, parenteral formulations usually include injectable fluids that are pharmaceutically and physiologically acceptable fluid vehicles such as water, physiological saline, other balanced salt solutions, aqueous dextrose, glycerol or the like. For solid compositions (e.g., powder, pill, tablet, or capsule forms), conventional non-toxic solid carriers can include, for example, pharmaceutical grades of mannitol, lactose, starch, or magnesium stearate. In addition to biologically-neutral carriers, pharmaceutical compositions to be administered can contain minor amounts of non-toxic auxiliary substances, such as wetting or emulsifying agents, preservatives, pH buffering agents, or the like, for example sodium acetate or sorbitan monolaurate. Excipients that can be included are, for instance, other proteins, such as human serum albumin or plasma preparations.

In some aspects, an agent that increases expression or activity of PDE4D3 is included in a controlled release formulation, for example, a microencapsulated formulation. Various types of biodegradable and biocompatible polymers can be used, and methods of encapsulating a variety of synthetic compounds, proteins and nucleic acids, have been described (see, for example, U.S. Patent Publication Nos. 2007/0148074; 2007/0092575; and 2006/0246139; U.S. Pat. Nos. 4,522,811; 5,753,234; and 7,081,489; PCT Publication No. WO/2006/052285; Benita, Microencapsulation: Methods and Industrial Applications, 2^{nd} ed., CRC Press, 2006).

In other aspects, an agent that increases expression or activity of PDE4D3 is included in a nanodispersion system. Exemplary nanodispersion systems and methods for producing such nanodispersions are provided in e.g., U.S. Pat. No. 6,780,324; U.S. Pat. Publication No. 2009/0175953. For example, a nanodispersion system includes a biologically active agent and a dispersing agent (such as a polymer, copolymer, or low molecular weight surfactant). Exemplary polymers or copolymers include polyvinylpyrrolidone (PVP), poly(D,L-lactic acid) (PLA), poly(D,L-lactic-co-glycolic acid (PLGA), poly(ethylene glycol). Exemplary low molecular weight surfactants include sodium dodecyl sulfate, hexadecyl pyridinium chloride, polysorbates, sorbitans, poly(oxyethylene) alkyl ethers, poly(oxyethylene) alkyl esters, and combinations thereof. In one example, the nanodispersion system includes PVP and ODP or a variant thereof (such as 80/20 w/w). In some examples, the nanodispersion is prepared using the solvent evaporation method, see for example, Kanaze et al., Drug Dev. Indus. Pharm. 36:292-301, 2010; Kanaze et al., J. Appl. Polymer Sci. 102:460-471, 2006.

With regard to the administration of nucleic acids, one approach to administration of nucleic acids is direct treatment with plasmid DNA, such as with a mammalian expression plasmid. As described above, the nucleotide sequence encoding a PDE4D3 protein (such as encoding SEQ ID NO: 2, or encoding a protein having at least 80%, at least 85%, at least 90%, at least 95%, at least 96%, at least 97%, at least 98%, at least 99%, or 100% sequence identity to SEQ ID NO: 2), can be placed under the control of a promoter, such as an adipocyte-specific promoter, to increase expression of the protein in adipose tissue.

Many types of release delivery systems can be used. Examples include polymer based systems such as poly(lactide-glycolide), copolyoxalates, polycaprolactones, polyesteramides, polyorthoesters, polyhydroxybutyric acid, and polyanhydrides. Microcapsules of the foregoing polymers containing drugs are described in, for example, U.S. Pat. No. 5,075,109. Delivery systems also include non-polymer systems, such as lipids including sterols such as cholesterol, cholesterol esters and fatty acids or neutral fats such as mono- di- and tri-glycerides; hydrogel release systems; silastic systems; peptide based systems; wax coatings; compressed tablets using conventional binders and excipients; partially fused implants; and the like. Specific examples include, but are not limited to: (a) erosional systems in which an agent that increases expression or activity of PDE4D3, such as a PDE4D3 protein (such as SEQ ID NO: 2, or a protein having at least 80%, at least 85%, at least 90%, at least 95%, at least 96%, at least 97%, at least 98%, at least 99%, or 100% sequence identity to SEQ ID NO: 2), a polynucleotide encoding such a protein, or a small molecule activator (such as MR-L2) is contained in a form within a matrix such as those described in U.S. Pat. Nos. 4,452,775; 4,667,014; 4,748,034; 5,239,660; and 6,218,371 and (b) diffusional systems in which an active component permeates at a controlled rate from a polymer such as described in U.S. Pat. Nos. 3,832,253 and 3,854,480. In addition, pump-based hardware delivery systems can be used, some of which are adapted for implantation.

Use of a long-term sustained release implant can be suitable for treatment of chronic conditions, such as diabetes. Long-term release, as used herein, means that the implant is constructed and arranged to deliver therapeutic levels of the active ingredient for at least 30 days, such as 60 days. Long-term sustained release implants include the release systems described above. These systems have been described for use with nucleic acids (see U.S. Pat. No. 6,218,371).

The dosage form of the pharmaceutical composition can be determined by the mode of administration chosen. For instance, in addition to injectable fluids, topical, inhalation, oral, and suppository formulations can be employed. Topical preparations can include eye drops, ointments, sprays, patches, and the like. Inhalation preparations can be liquid (e.g., solutions or suspensions) and include mists, sprays and the like. Oral formulations can be liquid (e.g., syrups, solutions or suspensions), or solid (e.g., powders, pills, tablets, or capsules). Suppository preparations can also be solid, gel, or in a suspension form. For solid compositions, conventional non-toxic solid carriers can include pharmaceutical grades of mannitol, lactose, cellulose, starch, or magnesium stearate.

### VIII. Summary of Several Aspects

Aspect 1. A method of reducing blood glucose in a subject, comprising:
administering to the subject a therapeutically effective amount of an agent that increases expression or activity of phosphodiesterase 4D isoform 3 (PDE4D3) in adipocytes of the subject, thereby reducing the blood glucose.
Aspect 2. A method of treating type 2 diabetes in a subject, comprising:
administering to the subject a therapeutically effective amount of an agent that increases expression or activity of phosphodiesterase 4D isoform 3 (PDE4D3) in adipocytes of the subject, thereby treating the type 2 diabetes.
Aspect 3. The method of aspect 1 or aspect 2, wherein the agent that increases expression or activity of PDE4D3 comprises a nucleic acid molecule encoding a PDE4D3 protein.
Aspect 4. The method of aspect 3, wherein the nucleic acid molecule encoding the PDE4D3 protein is operably linked to an adipocyte-specific promoter.
Aspect 5. The method of aspect 3 or aspect 4, wherein the nucleic acid molecule encoding the PDE4D3 protein comprises a vector.
Aspect 6. The method aspect 5, wherein the vector is a viral vector.
Aspect 7. The method of aspect 6, wherein the viral vector is an adenovirus vector or an adeno-associated virus (AAV) vector.
Aspect 8. The method of aspect 3, wherein the nucleic acid molecule encoding the PDE4D3 protein is introduced into adipocytes using a gene editing method.
Aspect 9. The method of aspect 8, wherein the gene editing method comprises clustered regularly interspaced short palindromic repeats (CRISPR)-CRISPR-associated protein 9 (Cas9), transcription activator-like effector nucleases (TALENs), or zinc-finger nucleases (ZFNs).
Aspect 10. The method of any one of aspects 3-9, wherein the PDE4D3 protein comprises the amino acid sequence set forth as SEQ ID NO: 1.
Aspect 11. The method of aspect 1 or aspect 2, wherein the agent that increases expression or activity of PDE4D3 is a small molecule activator of PDE4D3.
Aspect 12. The method of aspect 11, wherein the small molecule activator of PDE4D3 is an N-substituted-2-(3-aryl-1H-1,2,4-triazol-1-yl) acetamide.
Aspect 13. The method of aspect 12, wherein the small molecule activator of PDE4D3 is 2-(3-(4-chloro-3-fluorophenyl)-5-ethyl-1H-1,2,4-triazol-1-yl)-N-(3,5-dichlorobenzyl) acetamide (MR-L2).
Aspect 14. The method of any one of aspects 11-13, wherein the small molecule activator of PDE4D3 is conjugated to an antibody that specifically binds fibroblast growth factor receptor 1b (FGFR1b).
Aspect 15. The method of any one of aspects 11-13, wherein the small molecule activator of PDE4D3 is conjugated to an incretin.
Aspect 16. The method of aspect 15, wherein the incretin is gastric inhibitory peptide (GIP) or glucagon-like peptide-1 (GLP-1).
Aspect 17. The method of any one of aspects 14-16, wherein the small molecule activator of PDE4D is conjugated to the antibody or the incretin via an acid-sensitive linker.
Aspect 18. The method of any one of aspects 1-17, further comprising administering to the subject a therapeutically effective amount of a mature fibroblast growth factor 1 (FGF1) protein or a modified mature FGF1 protein.
Aspect 19. The method of aspect 18, wherein the modified mature FGF1 protein has reduced mitogenicity and/or increased stability compared to native FGF1 protein.
Aspect 20. The method of aspect 18 or aspect 19, wherein the modified mature FGF1 protein selectively binds FGFR1b.
Aspect 21. The method of any one of aspects 1-20, further comprising administering a therapeutically effective amount of an additional therapeutic compound.
Aspect 22. The method of aspect 21, wherein the additional therapeutic compound is insulin, an alpha-glucosidase inhibitor, amylin agonist, dipeptidyl-peptidase 4 (DPP-4) inhibitor, meglitinide, sulfonylurea, or a peroxisome proliferator-activated receptor (PPAR)-gamma agonist.
Aspect 23. The method of aspect 22, wherein the PPAR-gamma agonist is a thiazolidinedione (TZD), aleglitazar, farglitazar, muraglitazar, or tesaglitazar.
Aspect 24. The method of aspect 23, wherein the TZD is pioglitazone, rosiglitazone, rivoglitazone, or troglitazone.
Aspect 25. The method of any one of aspects 1-24, wherein the subject is a human, dog or cat.
Aspect 26. A nucleic acid molecule comprising a phosphodiesterase 4D isoform 3 (PDE4D3) coding sequence operably linked to an adipocyte-specific promoter.
Aspect 27. A vector comprising the nucleic acid molecule of aspect 26.
Aspect 28. The vector of aspect 27, which is a viral vector.
Aspect 29. The vector of aspect 27, which is a plasmid vector.
Aspect 30. A composition comprising a small molecule activator of phosphodiesterase 4D isoform 3 (PDE4D3) conjugated to:
an antibody that specifically binds fibroblast growth factor receptor 1b (FGFR1b); or
an incretin.
Aspect 31. The composition of aspect 30, wherein the incretin is gastric inhibitory peptide (GIP) or glucagon-like peptide-1 (GLP-1).
Aspect 32. The composition of aspect 30 or aspect 31, wherein the small molecule activator of PDE4D3 is 2-(3-(4-chloro-3-fluorophenyl)-5-ethyl-1H-1,2,4-triazol-1-yl)-N-(3,5-dichlorobenzyl) acetamide (MR-L2).
Aspect 33. A pharmaceutical composition, comprising a pharmaceutically acceptable carrier and the nucleic acid molecule of aspect 26, the vector of any one of aspects 27-29, or the composition of any one of aspects 30-32.

The following examples are provided to illustrate certain particular features and/or aspects. These examples should not be construed to limit the disclosure to the particular features or aspects described.

### EXAMPLES

### Example 1: Methods

This example describes the materials and methods used for the studies described in Examples 2-6.

### Mouse Model

Mice were kept in 12 h light/dark cycle in a temperature-controlled environment. Mice had free access to food and water unless noted otherwise. C57/B6 mice background was used unless noted otherwise. C57BL/6J and ob/ob (000632-B6. Cg-Lepob/J) male mice were obtained from Jackson Laboratory. PDE4D knock-out mice (034588-UCD) were obtained from MMRRC. For diet-induced obesity (DIO) studies, mice were fed a high fat diet (HFD) (60% fat, F3282; Bio-Serv) for minimum of 12 weeks to induce insulin resistance when diet-induced obese model is used. Insulin levels and blood glucose levels were monitored to confirm insulin resistance. To generate adipose-specific FGFR1 deletion, adiponectin-cre mice (B6; FVB-Tg (Adipoq-cre)1Evdr/J; Stock: 010803) were crossed to Fgfr1 flox/flox (B6.129S4-Fgfr1tm5.1Sor/J; Stock: 00767) mice. For AAV mediated expression of target proteins in adipose tissue, 5×10E+11 genomic copies of AAV were injected via tail vein or retro-orbital route.

### Cell Culture

All cells were grown at 37° C. in a 5% CO₂ humid atmosphere in DMEM, 10% FBS (GemCell, 100-500), 1× Antibiotic-Antimycotic (Gibco, 15240096) unless indicated otherwise. 3T3-L1 cells (ATCC, CL-173), mouse SVF-derived cells and human pre-adipocytes (Promocell, C-12735) were differentiated and treated as described below.

### Pharmacological Studies

Recombinant FGF1 (rFGF1) was dissolved in phosphate-buffered saline (PBS) at a concentration of 0.2 mg/ml and was injected subcutaneously at a 0.5 mg kg⁻¹ dose. Blood glucose was monitored at indicated time points after injection. For initial measurements (2-4 h) food was removed after injection to exclude any indirect effects stemming from anorexigenic effects of FGF1. PDE4 inhibitor roflumilast stock in DMSO was diluted at 1 mg/ml in 30% captisol, pH 10 and delivered to mice thorough oral gavage at 5 mg kg⁻¹ dose. PDE3 inhibitor cilostamide stock in DMSO was diluted at 2 mg/ml in 30% captisol and delivered through intraperitoneal injection at 10 mg kg⁻¹ dose.

### Metabolic Studies

Pyruvate tolerance and glycerol tolerance tests were performed in ob ob and DIO mice after overnight fasting at 1.5 g kg⁻¹ dose using sodium pyruvate (0.2 g/ml) or 20% glycerol solution in PBS. Insulin tolerance test and glucose tolerance test were performed in ob ob mice after 8 h fasting. 2 U kg⁻¹ insulin (Humulin R) and 0.5 g kg⁻¹ glucose were used respectively. Blood glucose from tail bleeding was monitored by a OneTouch glucometer.

### Hyperinsulinemic-Euglycemic Clamp Studies

Before the test, mice were equipped with a permanent catheter in the right atrium via the jugular vein and were allowed to recover over a period of at least 3 days. After the recovery period, the mice were placed in experimental cages. All infusion experiments were performed in conscious, unrestrained mice as described previously (van Dijk et al., 2003). During the experiment, blood glucose levels were determined every 15 minutes using a Lifescan EuroFlash glucose meter. For GC-MS analysis of [U-¹³C] glucose, bloodspots on filter paper were collected from the tail vein every 30 minutes.

### Hyperinsulinemic-Euglycemic Clamp Experiment

Ob/ob mice were treated for 1 week with rFGF1 (0.5 mg kg⁻¹ every other day for one week). Steady state glucose fluxes were determined for basal and hyperinsulinemic-euglycemic clamp conditions. During the first period, mice were infused with a solution containing a tracer of [U-13C] glucose (2.5 mg/ml Cambridge Isotope Laboratories, Andover, MA) at an infusion rate of 0.54 ml/h. With respect to the final period, blood glucose levels were clamped at 20 mM. For this, the mice were infused at a constant rate of 0.135 ml/h with a mixture of insulin (44 mU/ml, Actrapid, Novo Nordisk, Bagsvaerd, Denmark), somatostatin (20 μg/ml, UCB Breda, the Netherlands), 1% BSA, and glucose (200 mg/ml from which 3% [U-¹³C] glucose). Additionally, a second (variable) infusion was used containing glucose (200 mg/ml from which 3% [U-¹³C] glucose) to adjust blood glucose levels.

### Targeted Metabolomics

Polar metabolites were extracted and analyzed using a previously reported method (Yuan et al., 2012). Briefly, ob/ob mice were sacrificed by cervical dislocation. Liver pieces (50-100 mg) were snap frozen in liquid nitrogen. 1 ml LC-MS grade 80% methanol chilled at −80° C. was added per 100 mg tissue on dry ice. Samples were homogenized by TissueLyzer and lysates were incubated on dry ice for 30 minutes and centrifuged at 20000 g for 10 minutes. Clarified supernatants were transferred to new tubes. Pellets were extracted again and supernatants were combined. After drying the supernatant under nitrogen gas, extracts were dissolved in H₂O (40 μL) and 10 μL were subjected to liquid chromatography mass spectrometry (LC-MS). Polar metabolites were measured by LC-MS using a TSQ Quantiva instrument fitted with a Luna NH2 HPLC column (5.0 μm; 4.6 mm×50 mm, Phenomenex). The following LC solvents were used: buffer A, 95:5 H₂O/ACN, 20 mM ammonium hydroxide, 20 mM ammonium acetate; buffer B, 100% ACN. A typical LC run was 23 minutes long with a flow rate of 0.4 ml min-1 and consisted of the following steps: 85 to 30% buffer B over 3 minutes, 30 to 2% buffer B over 9 minutes, 2% buffer B for 3 minutes, 2 to 85% buffer B over 1 minute, and 85% buffer B for 7 minutes. MS analyses were performed using electrospray ionization (ESI) in negative or positive ion mode depending on the metabolites being analyzed. Negative mode and positive mode source parameters were the following: spray voltage 3.5 kV, ion transfer tube temperature of 325° C., and a vaporizer temperature of 275° C.

### Hepatic Pyruvate Carboxylase (Pcx) Activity Assay

Pcx activity was determined by malate dehydrogenase coupling method originally reported by Payne et al. (Payne and Morris, 1969). Briefly, mice were sacrificed by decapitation. Liver samples were rapidly frozen in liquid nitrogen within 5 seconds of excision designed to avoid loss of hepatic acetyl-CoA levels. Frozen liver samples were pulverized on dry ice and approximately 100 mg of tissues were homogenized with TissueLyzer in Pcx activity assay buffer (50 mM Tris pH8, 10 mM MgCl₂, 10 mM NaHCO₃). Homogenates were cleared by centrifugation at 14000 rpm for 10 minutes. Cleared supernatants were diluted in Pcx activity assay buffer to approximately 1 μg/ul. Approximately 5 μg protein was loaded onto 96-well plates and the total volume was brought up to 20 ul by Pcx activity assay buffer. Absorbance at 340 nm was monitored every 9 seconds at 37° C. immediately after the addition of 80 ul Pcx reaction buffer (50 mM Tris pH8, 10 mM MgCl₂, 10 mM NaHCO₃, 6.25 mM ATP, 0.125 mM NADH, 2.5 mM pyruvate, malate dehydrogenase 0.025 U/ml in Pcx activity assay buffer). Pcx activity was determined as the loss of absorbance at 340 nm over time normalized to protein concentration.

### Hepatic Metabolites Quantification

Liver samples were prepared from HFD-fed mice as described in Pcx activity assay. After determining Pcx activity from sample homogenates, the remaining supernatant was de-proteinated by perchloric acid (PCA)-KOH method using a commercial kit. De-proteinated samples were used to quantify hepatic metabolites using the commercial kits. Hepatic metabolites concentration was normalized to protein concentration and corrected for the loss of volume due to de-proteinization.

### Adipose Transplantation

8-week old F1WT mice were sacrificed. Their gonadal adipose tissue (gWAT) was excised into small pieces (approximately 3 mm×3 mm) and maintained in saline briefly. Age-matched 8-week old F1WT and FIKO mice were anesthetized by ketamine/xylazine solution (80 mg kg⁻¹ and 10 mg kg⁻¹, respectively). Multiple small incisions in dorsum were made in anesthetized mice and a piece of gWAT from F1WT was placed inside each incision. Each recipient mouse received the entire gWAT from the donor mouse. Wounds were closed by wound clip. Mice were allowed to recover on heat pad. Mice were monitored, and antibiotics and pain medicine (ibuprofen) were provided post-surgery for 3 days. 2 weeks after surgery, recipient mice were sacrificed.

### Isolation of Adipose Stromal Vascular Fraction

Isolation of adipose stromal fraction (SVF) was performed as published previously with slight modifications (Bapat et al., 2015). Inguinal adipose (iWAT) tissues were used for isolation of SVF due their ability to differentiate to mature adipocytes. Briefly, adipose tissues were dissected, washed in cold PBS and cut into small pieces in digestion buffer (100 mM HEPES, 120 mM NaCl, 50 mM KCl, 1 mM CaCl₂), 1.5% fatty acids free BSA, 1 mg/ml collagenase I). Samples were dissociated in 37° C. water bath with shaking for 30-60 min with occasional monitoring to prevent over digestion. Tissue debris were filtered by 100 μm cell strainer and SVF was collected by centrifugation at 500 g for 5 minutes. Cell pellet was washed once by PBS and filtered with 40 μm cell strainer. Red blood cells were lysed with Red blood cell lysis buffer (BioLegend) according to manufacturer's instructions. The remaining cells were re-suspended and cultured in DMEM/F12 with 20% FBS (GemCell, 100-500) at 37° C., 5% CO₂.

### In Vitro Differentiation of Adipocytes

Differentiation of pre-adipocytes were based on previously published methods (Bunnell et al., 2008). Briefly, 3T3-L1 pre-adipocytes were grown to full confluence in DMEM, 10% FBS (GemCell, 100-500), 10 mM HEPES and antibiotic-antimycotic (full growth media). 2 days later, differentiation was induced by replacing the media with 1 μM dexamethasone, 1 μM rosiglitazone, 500 μM IBMX and 5 μg/ml insulin in full growth media 2 days later after reaching full confluence. Two days later media was replaced by differentiation induction media with 1 μM rosiglitazone and 5 μg/ml insulin for 4 days with media change after 2 days. Cells were kept in maintenance medium (full growth media with 5 μg/ml insulin) for 4 more days for full differentiation. For differentiation of mouse SVF derived adipocytes, the protocol was same except DMEM/F12 (10565018, ThermoFisher) was used as base media and initial differentiation induction media was kept for 3 days. For differentiation of human subcutaneous adipocytes, protocol and media from the manufacturer (C-12735, C-39437, Promocell) were used. Dexamethasone was omitted from the media in the last 3 days before performing experiments. Both mouse and human SVF derived adipocytes were differentiated in collagen coated plates (A1142802, ThermoFisher).

### Lipolysis Assays

For in vitro lipolysis assay, cells were washed with PBS and media were changed to full growth media 1 day before experiment. Cells were serum starved for 2 h and placed in KRBH buffer (30 mM HEPES, 120 mM NaCl, 4 mM KH₂PO₄, 1 mM MgSO₄, 0.75 mM CaCl₂), and 10 mM NaHCO₃) with 2% fatty acid free BSA and 5 mM glucose. FGF1 was added 10 min before induction of lipolysis with 100 nM isoprotenol (ISO). Inhibitors were added 30 min prior to FGF1 treatment unless otherwise noted. Media were collected and cells were lysed in protein extraction buffer. FFAs were measured in the media using a commercial kit (Wako-NEFAHR2) and normalized to protein concentration. For ex vivo lipolysis, the assay was modified from a published protocol (Funicello et al., 2007). Briefly, approximately 0.1 gram of adipose tissue was excised, weighed and kept in cold PBS until treatment and control tissues were collected. Tissues were cut into small pieces and incubated in same KRBH buffer as described above for 4 h. FFAs were measured and normalized by the explant weights.

### Protein Extraction and Immunoblotting

Tissues were lysed in cold lysis buffer (50 mM Tris, pH 7.5, 150 mM NaCl, 1% NP40, 0.5% NaDoc, 0.1% SDS, 5% glycerol, 1 mM EDTA, protease and phosphatase inhibitors (complete and PhosSTOP (Roche)) by homogenization by bead-beater for 30 s. Samples were cleared for 10 min at 18,000 g at 4° C. and the middle clear phase was transferred to new tubes. A second 30 min centrifugation was performed and the middle clear phase was transferred to new tubes. Five ul protein extract was used for BCA assay to determine protein concentration. Samples were boiled in Laemmli buffer. SDS-PAGE and blotting were performed using gradient gels and Trans-Blot Turbo Transfer System (Bio-Rad). Antibodies used were PDE4D (12918-1-AP, Proteintech), pHSL-660 (4126S, Cell signaling), HSL (4107S, Cell Signaling), tubulin (CP06, Millipore Sigma), and GFP (A01388, GenScript). PDE4D3-pS44 phospho-specific antibody was generated using PDE4D3-S44 FRRHpSWISFDVDNGTSAGR peptide (SEQ ID NO: 20) (Mackenzie et al., 2002) by Pocono Rabbit Farm, using 70-day rabbit antibody production protocol and purified via affinity purification. Primary antibodies were incubated for 2 h at room temperature (RT) or overnight (ON) at 4° C. Blots were developed using SuperSignal West Pico PLUS Chemiluminescent Substrate (ThermoFisher Scientific) and imaged with BioRad GelDoc system.

### Molecular Cloning and Production of Adenoviral/Adeno-Associated Virus

Virapower gateway adenovirus expression kit was used to clone ORFs of various isoforms of mouse PDE4D (XM_006517645.4, XM_030247262.1 and XM_006517647.3) to adenoviral vectors according to the manufacturer's protocol. Vectors were transfected into 293A cell line using Fugene transfection reagent (PRE2692, Promega). Crude adenovirus stock was used to infect new 293A cells for large-scale adenovirus production. Transduced 293A cells were harvested when cytopathic effect was apparent 2-3 days after inoculation with crude adenovirus stock. Virus were purified via adenovirus standard purification kit (3054, Virapur). For adipose specific expression of target proteins by AAV mediated transduction (adAAV), the vector was designed by using a human adiponectin promoter/enhancer (SEQ ID NO: 27) based on O'Neil et al. (O'Neill et al., 2014) and 4× repeat miRNA target for miR 122T (SEQ ID NO: 28) (Qiao et al., 2011) after cloned ORF to further prevent liver expression. adAAV backbone was synthesized by Vector Builder. ORFs of PDE4D3, GFP, mCherry, Perilipin-GFP, HSL-mCherry were cloned to the vector with standard restriction digestion cloning. cDNA from gonadal adipose tissue was used to amplify the ORFs with the primers listed at Table 1. Stabl3 cells were used for vector amplification and Hi Pure Purelink Expi Plasmid Giga Prep (ThermoFisher) was used for AAV vector purification. Large scale AAV8 production and calculation of the titer were performed by Salk Institute Gene Transfer, Targeting, and Therapeutics Core (GT3). Mutagenesis experiments were performed by using QuickChange XL Site-Directed Mutagenesis Kit (200517, Agilent). Primer sequences are listed at Table 1.

TABLE 1
Primers

| | SEQ |
|---|---|
| | ID |
| Primer Name and Sequence | NO: |
| PDE4D3 frw (for adenoviral cloning): cacc ATG CTC AAA CCA GAG TGT TGG | 3 |
| GA | |
| PDE4D4 frw (for adenoviral cloning): cacc ATG GCT TTT GTC TGG GAT CCC | 4 |
| PDE4D5 frw (for adenoviral cloning): cacc ATG AGC ATT ATA ATG AAG CCA | 5 |
| AGA TCC | |
| PDE4D rev (for adenoviral cloning): TTA CGT ATC AGG ACA GCA GTC ATC | 6 |
| PDE4D3 frw (for ad AAV cloning): | 7 |
| aaaGATATCCAAGTTTGTACAAAAAAGCAGGCTGCCACCATGCTCAAACCA | |
| GAGTGTTG | |
| PDE4D3 rev (for ad AAV cloning): aaaCATATGTTACGTATCAGGACAGCAGTC | 8 |
| GFP frw (for ad AAV cloning): | 9 |
| aaaGATATCCAAGTTTGTACAAAAAAGCAGGCTGCCACCATGGTGAGCAAG | |
| GGCGAGGA | |
| GFP rev (for ad AAV cloning): aaaCATATGTTACTTGTACAGCTCGTCCATG | 10 |
| Lipe (HSL) frw (for ad AAV cloning): aaa GATATC ACTAGT | 11 |
| CAAGTTTGTACAAAAAAGCAGGCTGCCACC | |
| ATG GAT TTA CGC ACG ATG ACA CAG | |
| Lipe (HSL) rev (for ad AAV cloning): aaa ACGCGTGTT CAG TGG TGC AGC | 12 |
| AGG CG | |
| mCherry frw (for ad AAV cloning): aaa GATATC ACTAGT ACGCGT GGATCC | 13 |
| ATGGTGAGCAAGGGCGAGG | |
| mCherry rev (for ad AAV cloning): aaa CATATG | 14 |
| TCACTTGTACAGCTCGTCCATG | |
| Perilipin GFP frw (for ad AAV cloning): aaa GATATC ACTAGT | 15 |
| CAAGTTTGTACAAAAAAGCAGGCTGCCACC | |
| ATGTCAATGAACAAGGGCCCAA | |
| Perilipin GFP rev (for ad AAV cloning): aaa CATATG | 16 |
| TTAAACTCTTTCTTCACCGGCAT | |
| PDE4D3-S44A frw (For point mutagenesis): cttccctttcagaaggcatgcctggatatgtttcga | 17 |
| PDE4D3-S44A rev (For point mutagenesis): tcgaaacatatccaggcatgccttctgaaagggaag | 18 |
| PDE4D3-S85A frw (For point mutagenesis): tcaacgccgggaggccttcctgtaccg | 19 |

### PDE Assay

PDE activities were measured by using [3H]-labelled cAMP as described previously with slight modifications (Rybalkin et al., 2013). Briefly, 10 μg of protein extract was incubated in assay buffer (20 mM Tris pH 7.4, 0.8 mM EGTA pH 8, 0.2 mg/ml BSA, 15 mM magnesium acetate, 1 μM cAMP, 50000 cpm [3H]-cAMP) in 250 ul volume at 30° C. for 15 min. Reaction was stopped by adding 125 ul, 0.25M HCl and neutralized by adding 125 ul 0.25M NaOH and final 100 mM Tris-HCl pH 7.4. Five ul Crotalus atrox venom (BML-KI307) was used for dephosphorylation of [3H]-5-AMP at 30° C. for 30 min. [3H]-adenosine product was separated from [3H]-cAMP substrate by ion-exchange chromatography (DEAE-Sephadex A-25; GE Healthcare) and quantified by scintillation counting.

### Microscopy

3T3-L1 cells were transduced with viral particles (10+-106GC/cell) in 24 well plates. Media was replaced after overnight incubation. 3-4 days after AAV infection, cells were treated as described for lipolysis assay. For cAMP analysis by biosensors, Downward Green cADDis cAMP Sensor (D0200G) and control mNeon Green (F0500G) produced in BacMam system was purchased from Montana Molecular. Brightfield and fluorescence images were taken every 10-20 min in I IncuCyte® Live-cell analysis system (Sartorius) and images were analyzed by IncuCyte® Analysis Software. High-resolution live cell imaging was performed with LSM 880 Airyscan microscope at 40× objective.

### Replication and Randomization

Animal experiments were performed on multiple cohorts. In vitro experiments were performed at least 3 times. The randomized block design was used for all animal experiments. The age, sex, body weight and cage effect were identified as blocking factors. Therefore, all animal experiments were carried out on age-matched animals of the same sex. Body weights were measured before assigning treatment groups. Cage effect was controlled in pharmacological treatment studies by assigning animals to the placebo or treatment group from the same cage.

### Quantification and Statistical Analysis

Pre-determined sample exclusion criterion was established for technical failures. Unless otherwise noted, statistical significance was calculated by unpaired, two-tailed student's t test. In time series data, two-way ANOVA was performed. Data are presented as mean±SEM.

### Example 2: FGF1 Suppresses Adipose Lipolysis in an Adipose FGFR1-Dependent Fashion

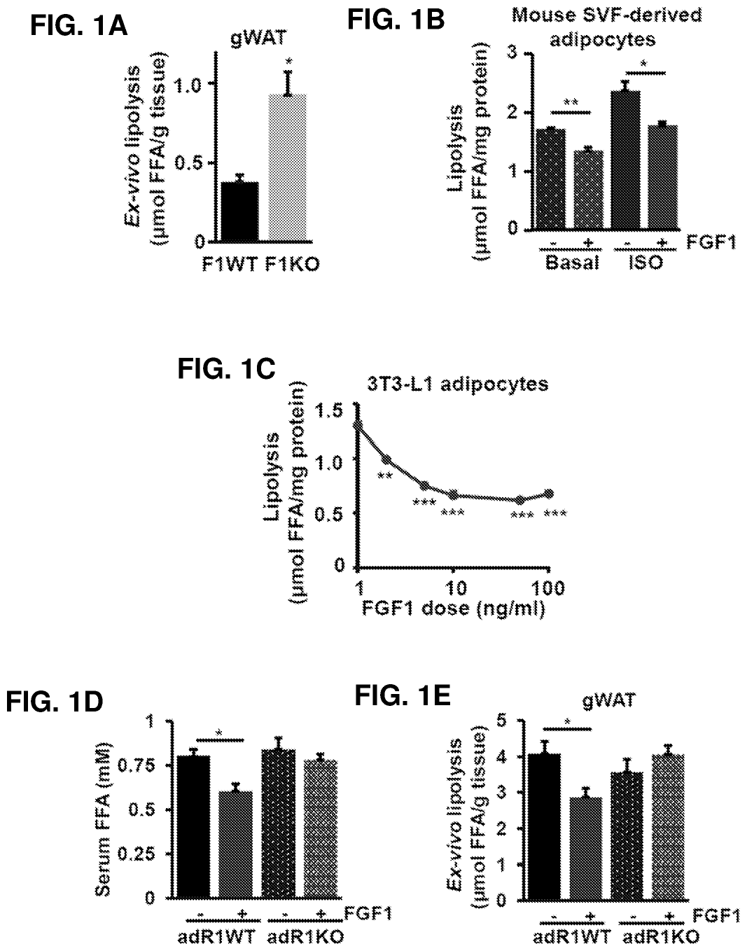

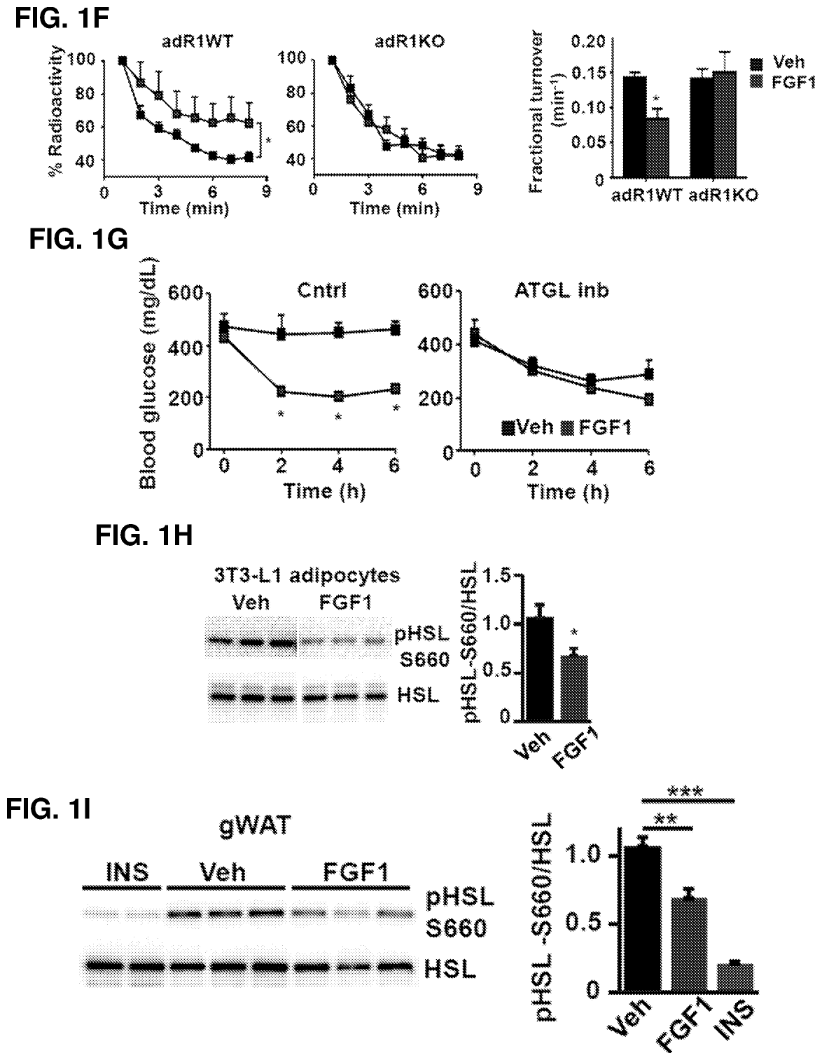

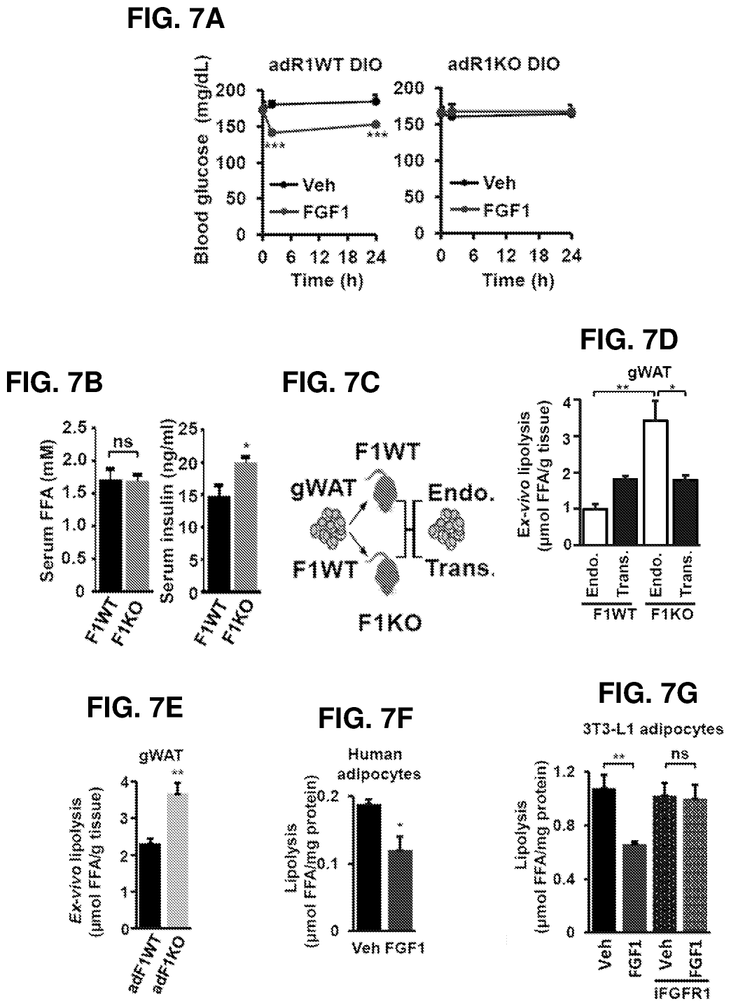

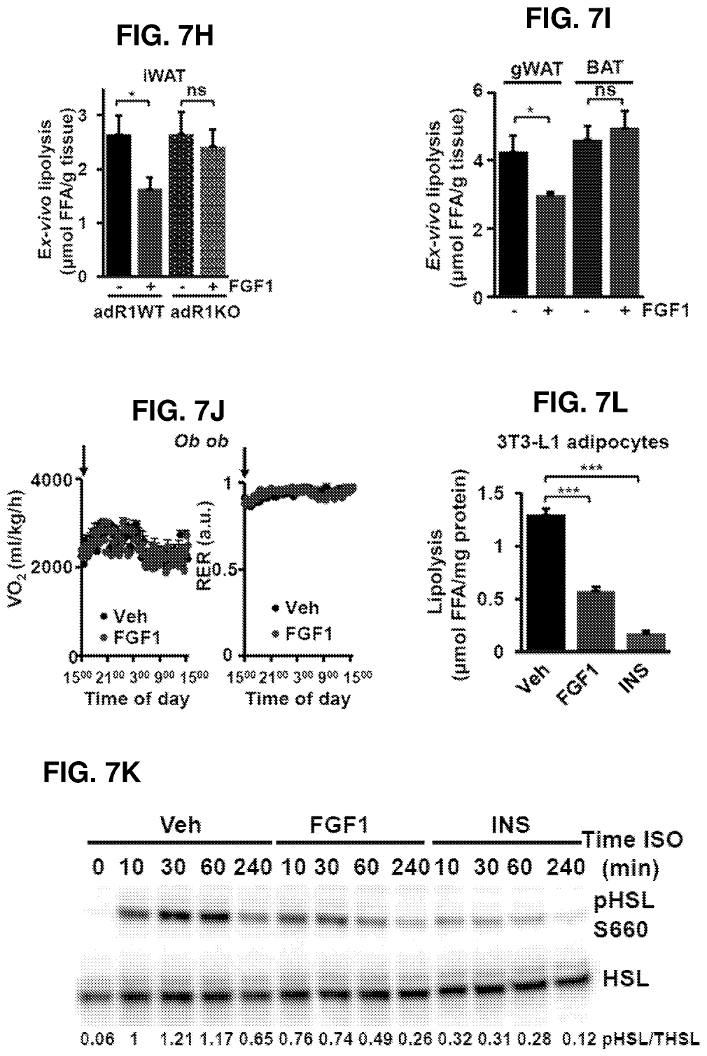

To validate that FGF1-induced glucose lowering is dependent on FGFR1 expression in adipose tissue, FGFR1 was selectively deleted in mature adipocytes (Fgfr1 f1/f1 crossed to adiponectin-CRE, adR1KO mice). FGF1 rapidly decreased blood glucose levels in diet-induced obese (DIO) wild type mice (0.5 mg/kg FGF1 s.c., adR1WT) but failed to affect adR1KO mice, consistent with previous findings (Suh et al., 2014) (FIG. 7A). Given the increase in insulin levels in adR1KO mice (Table 2), and the link between hepatic glucose production (HGP) and lipolysis, it was hypothesized that FGF1 may affect adipose lipolysis (Perry et al., 2015a; Perry et al., 2018; Rebrin et al., 1996). To explore this possibility, it was first determined whether lipolysis was perturbed in FGF1 knockout (FIKO) mice. While no differences were seen in serum FFA levels in fed mice, the higher serum insulin levels in FIKO mice suggested a dysregulated lipolytic response compensated by insulin (FIG. 7B). Indeed, ex vivo lipolysis assays in the absence of insulin compensation revealed markedly elevated lipolysis in the gonadal adipose tissue (gWAT) of FIKO mice (FIG. 1A). The persistence of the increased ex vivo lipolysis in FIKO gWAT in an adipose transplant model was indicative of an adipose-autonomous effect (FIGS. 7C and 7D). Mice harboring the selective deletion of FGF1 in adipose tissue (Fgf1 f1/f1 crossed to adiponectin Cre) showed a similar increase in gWAT ex vivo lipolysis (FIG. 7E). In addition, FGF1 acutely suppressed basal and isoproterenol-induced lipolysis in mouse and human stromal vascular fraction (SVF)-derived adipocytes, consistent with an adipocyte-intrinsic effect (FIG. 1B and FIG. 7F). Similarly, FGF1 dose-dependently suppressed isoproterenol-induced lipolysis in 3T3-L1 adipocytes (FIG. 1C), an effect that was blocked by FGFR1 inhibition (FIG. 7G).

TABLE 2
Phenotypic characterization of HFD-fed adR1KO mice (upper panel)
and serum leptin levels 30 min after FGF1 injection (lower panel)

| | adR1WT (n = 7) | adR1KO (n = 6) |
|---|---|---|
| Body Weight (g) | 47.41 ± 0.76 | 48.50 ± 1.40 |
| Glucose (mg/dL) | 202.42 ± 4.49 | 198.83 ± 8.82 |
| Insulin (ng/mL) | 11.64 ± 0.77 | 15.05 ± 1.13* |
| Leptin (ng/ml) | 107.55 ± 6.43 | 115.51 ± 9.03 |
| | Veh | FGF1 |
| Chow -fed serum | 1.00 ± 0.19 | 0.95 ± 0.15 |
| leptin (ng/ml) | (n = 5) | (n = 5) |
| HFD-fed serum leptin | 115.37 ± 13 | 121.25 ± 6 |
| (ng/ml) | (n = 4) | (n = 5) |

To determine whether exogenous FGF1 can similarly affect adipose lipolysis in vivo, DIO adR1WT and adR1KO mice were fasted overnight to minimize compensatory changes in insulin prior to injection with FGF1 (0.5 mg/kg s.c.). FGF1 reduced serum FFA levels in adR1WT mice by ˜30%, but failed to affect adR1KO mice (FIG. 1D). Moreover, ex vivo lipolysis was suppressed by FGF1 in an adipose FGFR1-dependent manner (FIG. 1E and FIG. 7H). As a measure of in vivo lipolysis, adR1WT and adR1KO mice pretreated with and without FGF1 were portally-infused with radiolabeled oleic acid. The fractional turnover rate of oleic acid was reduced in FGF1-treated adR1WT mice, indicative of lower basal lipolysis. In contrast, oleic acid turnover in adR1KO mice was not affected by FGF1 pretreatment (FIG. 1F).

The above findings implicate FGF1-FGFR1 signaling as a novel pathway regulating adipose lipolysis. This regulation appears specific to white adipose depots, as FGF1 did not affect lipolysis in brown adipose tissue (FIG. 7I). Moreover, FGF1 did not alter whole body metabolism, or affect circulating levels of leptin (FIG. 7J, Table 2). To determine the contribution of lipolytic regulation to FGF1-mediated glucose lowering, lipolysis was pharmacologically blocked with atglistatin, an inhibitor of adipose triglyceride lipase (ATGL). Atglistatin (120 mg/kg p.o) rapidly lowered blood glucose levels in ad lib fed ob/ob mice, consistent with the indirect regulation of hepatic glucose production by the products of lipolysis (Perry et al., 2015a). While as a single agent, FGF1 (0.5 mg/kg s.c.) robustly lowered blood glucose, no additive effects were seen when FGF1 was co-administered with atglistatin, supporting the notion that exogenous FGF1 lowers blood glucose by suppressing lipolysis (FIG. 1G). The rapid in vivo kinetics suggested that FGF1 may regulate lipolysis posttranslationally. To explore this possibility, the ability of FGF1 to affect PKA-mediated activation of HSL was determined. In 3T3-L1 adipocytes, FGF1 suppressed HSL phosphorylation at S660 (pHSL) under both basal and isoproterenol (ISO)-stimulated conditions (FIG. 1H and FIG. 7K). Similarly, pHSL levels in gWAT were decreased 30 min after FGF1 injection (FIG. 1I). Moreover, the in vivo suppression of HSL phosphorylation upon FGF1 treatment correlated with the in vitro suppression of lipolysis (FIG. 7L).

### Example 3: FGF1 Regulates Hepatic Glucose Production (HGP)

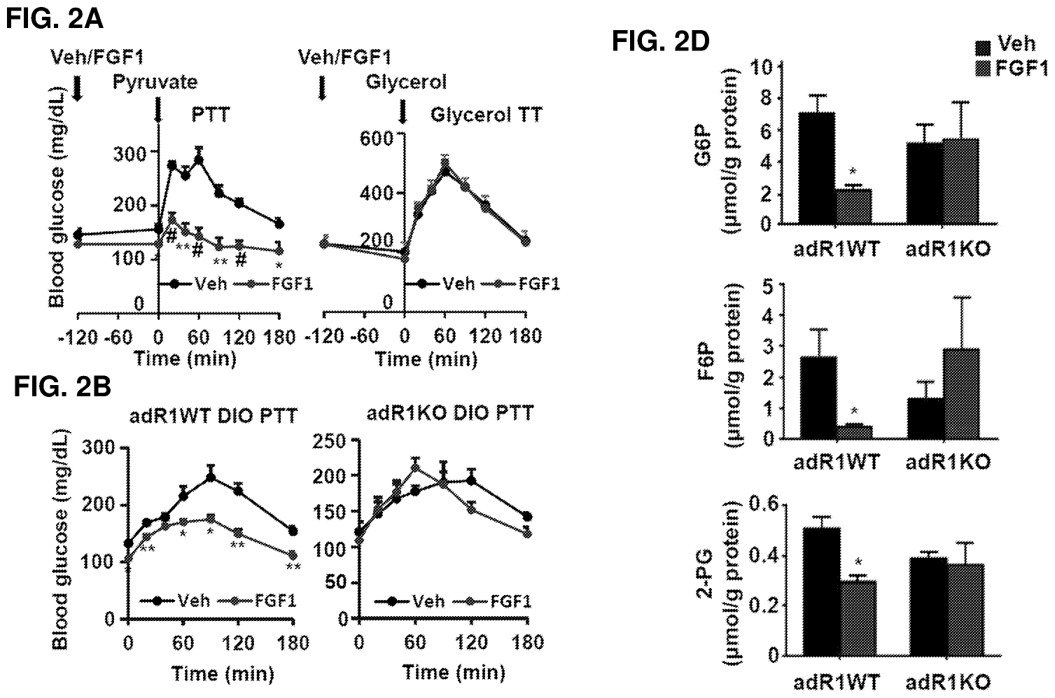

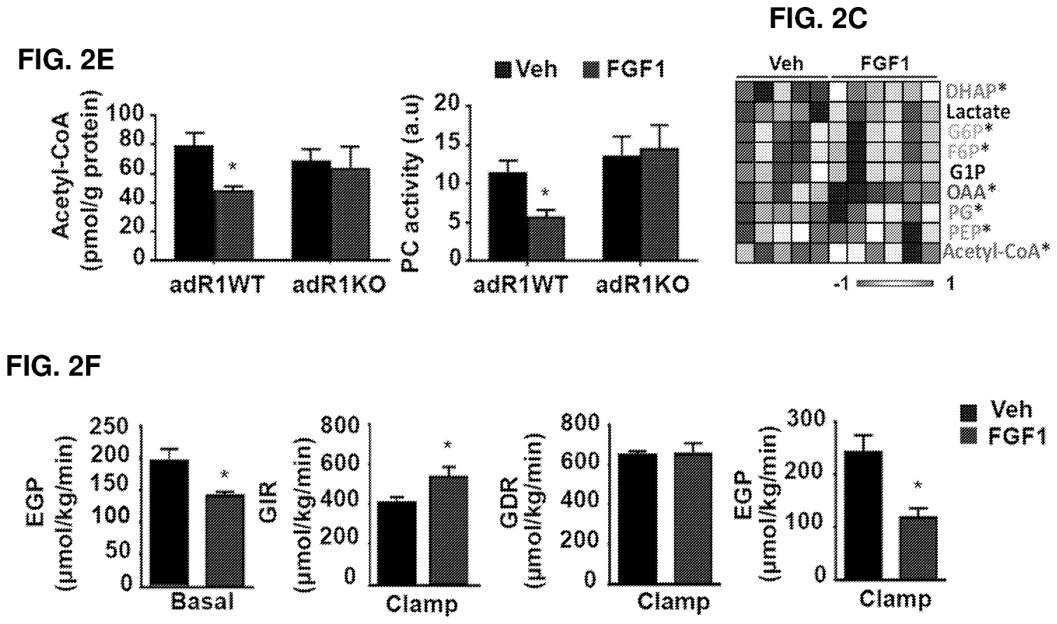

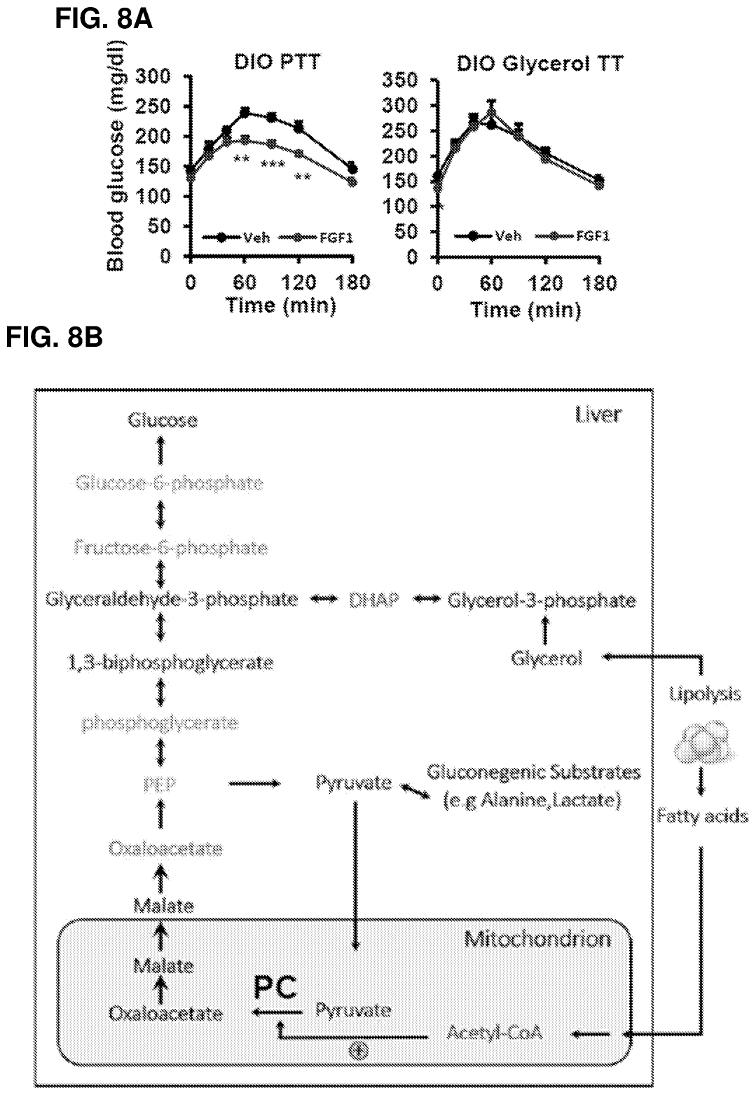

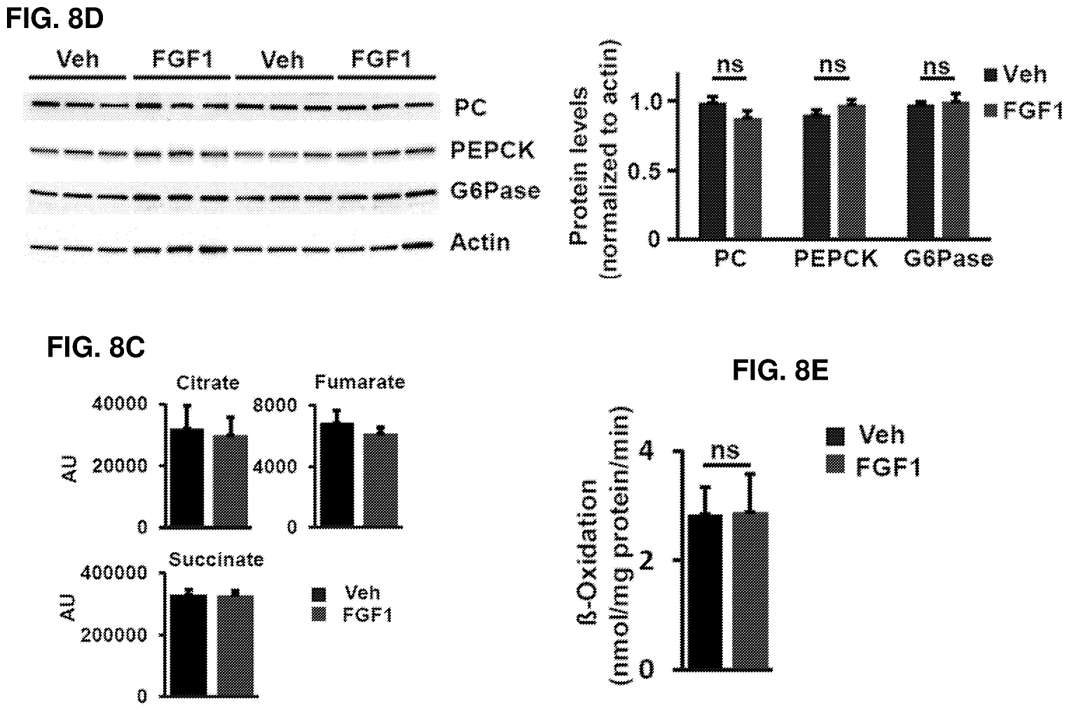

Insulin regulates blood glucose levels in part by suppressing lipolysis and thereby HGP, and dysregulated HGP contributes to hyperglycemia in insulin resistance (Boden et al., 2017; Lin and Accili, 2011; Lombardo and Menahan, 1979; Turner et al., 2005). To determine whether the suppression of lipolysis by FGF1 acutely reduced HGP, the ability of FGF1 to affect gluconeogenic substrate utilization was measured. oblob mice pretreated with FGF1 had a markedly reduced ability to synthesize glucose from pyruvate (pyruvate tolerance test; PTT), while no differences were seen when glycerol was the exogenous substrate (glycerol tolerance test; Glycerol TT) (FIG. 2A). These findings were replicated in DIO mice (FIG. 8A). Moreover, the ability of FGF1 to suppress pyruvate utilization was dependent on adipocyte FGFR1 expression (FIG. 2B). This differential sensitivity of pyruvate and glycerol utilization localized the effect of FGF1 on gluconeogenesis to a step downstream of pyruvate (Exton and Park, 1967; Shrago and Lardy, 1966) (FIG. 8B). To further delineate the FGF1-sensitive step, the levels of gluconeogenic intermediates were measured by mass spectrometry in the livers of oblob mice 2 h after FGF1 injection. Intermediates downstream of pyruvate including glucose 6-phosphate (G6P), fructose 6-phosphate (F6P), phosphoglycerate (PG), phosphoenolpyruvate (PEP), and oxaloacetate (OAA) were reduced in FGF1 injected mice, whereas metabolites involved in the tricarboxylic acid cycle (TCA) cycle were not affected (FIG. 2C and FIG. 8C). These reductions in substrate levels occurred in the absence of changes in protein expression in the rate-limiting enzymes involved in gluconeogenesis (FIG. 8D). In addition, levels of the allosteric activator of pyruvate carboxylase, acetyl-CoA were decreased (FIG. 2C). These reductions in gluconeogenic substrates were largely recapitulated in adR1WT mice, whereas adR1KO mice were insensitive to FGF1 treatment (FIG. 2D). Furthermore, an adipose FGFR1-dependent reduction in acetyl-CoA was accompanied by ˜50% reduction in pyruvate carboxylase activity (FIG. 2E). Acetyl-CoA is the product of fatty acid β-oxidation, hence the absence of any change in hepatic β-oxidation (FIG. 8E) supports a mechanism in which the reduction of free fatty acids upon FGF1 treatment decreases the activity of pyruvate carboxylase and thereby, limits HGP.

To test the relevance of these findings to glucose homeostasis, a hyperinsulinemic clamp was performed on ob/ob mice after short-term serial FGF1 administration (0.5 mg/kg every other day for a week). This limited treatment regimen resulted in a ˜25% reduction in basal endogenous glucose production (EGP) (FIG. 2F). Under clamp conditions, a higher exogenous glucose infusion rate (GIR) was required to maintain the glucose set-point in FGF1-treated mice; an effect largely attributed to diminished EGP as the glucose disposal rate (GDR) was not altered (FIG. 2F).

### Example 4: FGF1 Activation of PDE4 Inhibits cAMP-PKA Pathway

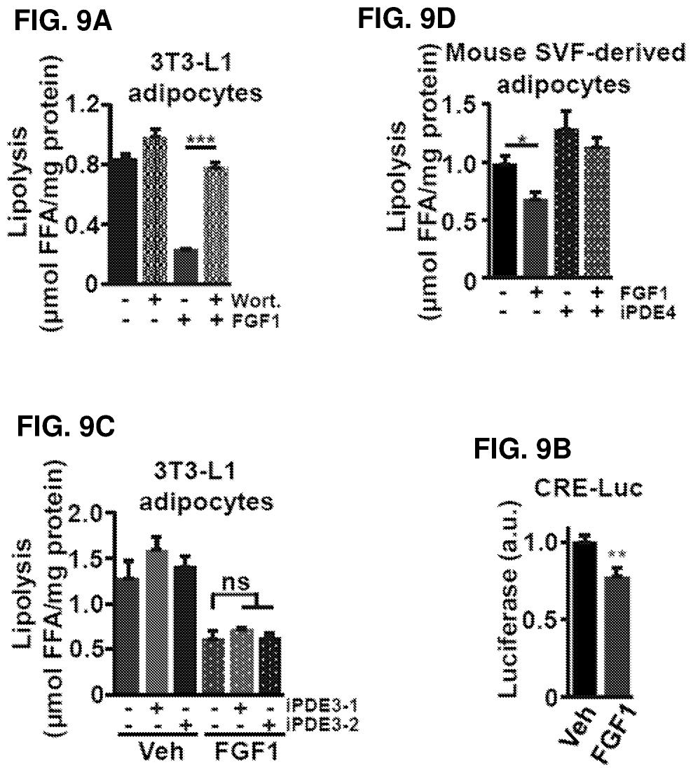

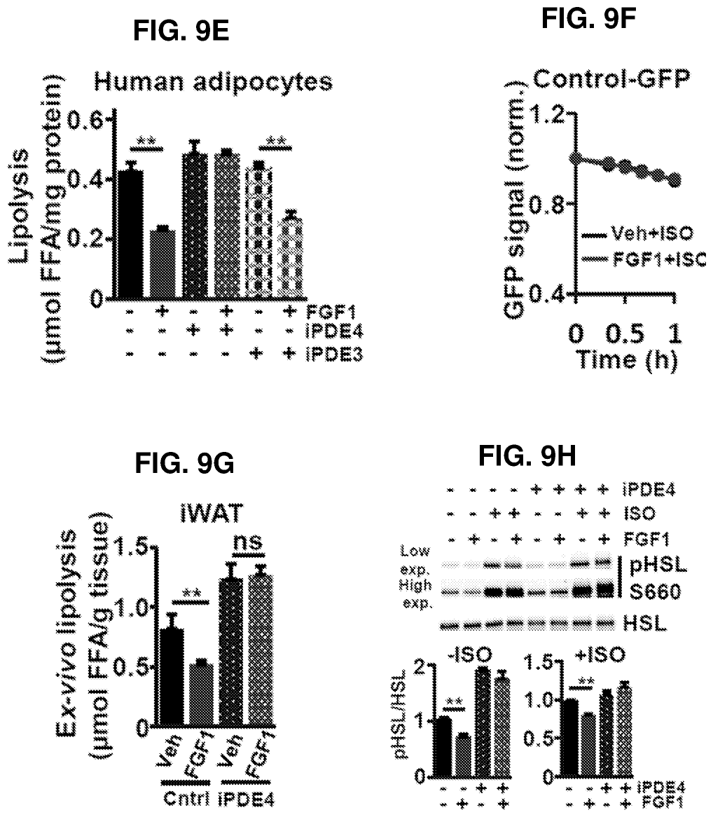

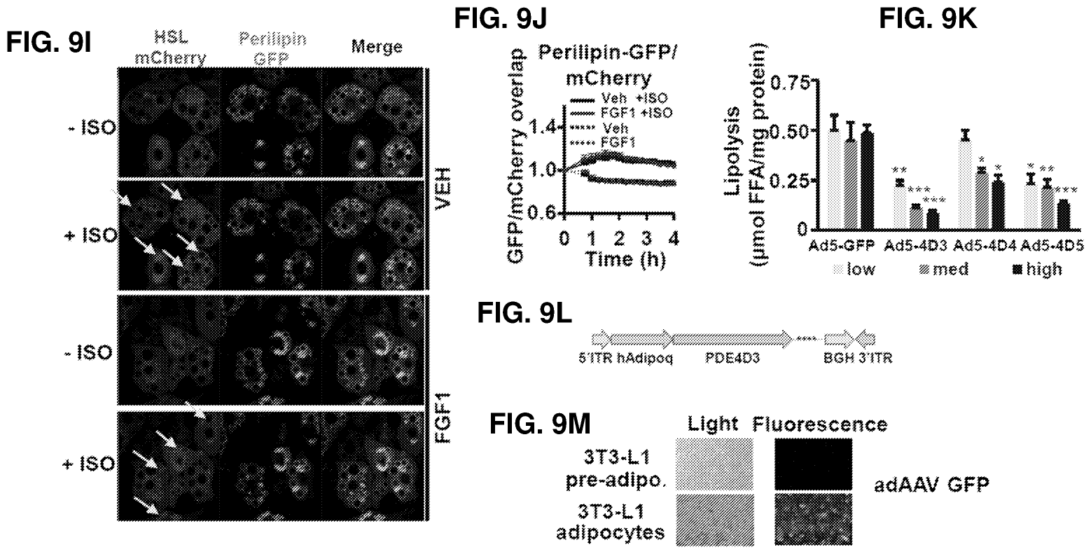

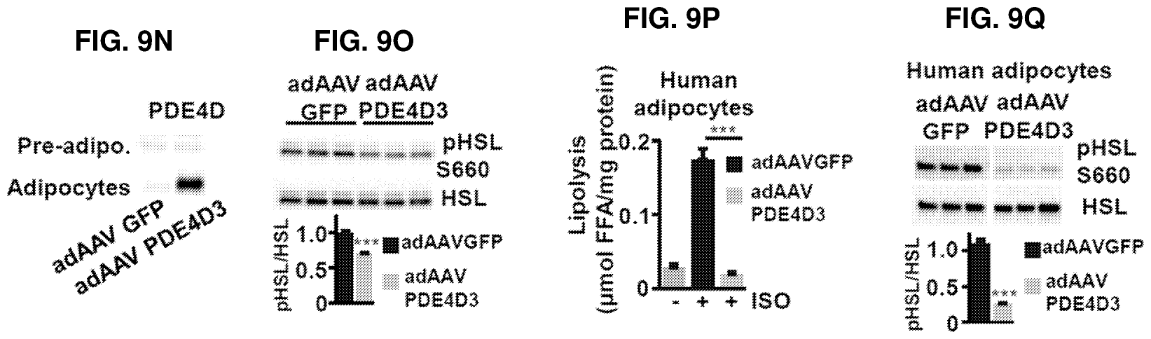

Insulin suppresses lipolysis via the PI3K-dependent activation of PDE3B (DiPilato et al., 2015a; Rahn et al., 1994). As FGFR1 activation can also signal via the PI3K pathway, it was investigated whether the anti-lipolytic effects of FGF1 were affected by the PI3K inhibitor wortmannin. Paralleling insulin signaling, wortmannin abrogated the FGF1-induced reduction in FFA release in 3T3-L1 adipocytes (FIG. 9A). In addition, FGF1 attenuated isoproterenol-induced increases in cAMP and cAMP/PKA signaling in a CRE-luciferase-based reporter system, implicating a possible effect on phosphodiesterase activity (FIG. 3A and FIG. 9B). Inhibition of PDE3B did not impair FGF1-induced suppression of lipolysis (FIG. 9C). In contrast, the anti-lipolytic activity of FGF1 was blocked by selective inhibitors of PDE4 in 3T3-L1 adipocytes, as wells as in mouse and human SVF-derived adipocytes (FIGS. 3B, 9D and 9E). Moreover, the FGF1-induced attenuation of an isoproterenol-driven increase in cAMP was lost in the presence of a PDE4 inhibitor, as measured in live 3T3-L1 cells using a fluorescence-based cAMP Biosensor (Tewson et al., 2016) (FIG. 3C). No FGF1 effects were seen in cells expressing the GFP control (FIG. 9F). To determine whether PDE4 activity was required for FGF1-induced suppression of lipolysis in vivo, DIO mice were gavaged with a PDE4 inhibitor 1 h prior to FGF1 injection. Analyses of adipose explants from those mice demonstrated that PDE4 inhibition blocked the ability of FGF1 to suppress lipolysis (FIG. 3D, FIG. 9G).

Given the above findings, it was posited that FGF1-PDE4 signaling regulated HSL phosphorylation. Indeed, in both basal and isoproterenol-stimulated cells, the ability of FGF1 to suppress HSL phosphorylation was lost in the presence of the PDE4 inhibitor roflumilast (FIG. 9H). The translocation of phosphorylated HSL to the lipid droplet and its subsequent interaction with perilipin is a key regulatory step in lipolysis (Clifford et al., 2000; Egan et al., 1992; Greenberg et al., 1991; Shen et al., 2009). To monitor the ability of FGF1 to affect pHSL-perilipin interactions in live cells, adeno-associated virus (AAV) vectors incorporating the human adiponectin promoter/enhancer to restrict expression to mature adipocytes (adAAVs) were used to express GFP-tagged perilipin (perilipin-GFP) and mCherry-tagged HSL (HSL-mCherry) in 3T3-L1 adipocytes (O'Neill et al., 2014). FGF1 reduced the isoproterenol-induced co-localization of perilipin-GFP and HSL-mCherry, as seen in the temporal monitoring of fluorescence overlap and by confocal microscopy (FIG. 3E left panel and FIG. 91). No effects were seen in cells expressing perilipin-GFP and mCherry without the HSL fusion (FIG. 9J). Moreover, while selective inhibition of PDE4 or PDE3 increased perilipin-HSL co-localization, consistent with increased cAMP levels and PKA activation, only PDE4 inhibition abrogated the FGF1 effect (FIG. 3E, middle and right panels).

In combination, these findings suggested a model in which FGF1-FGFR1 activation of PDE4 attenuates cAMP/PKA phosphorylation of HSL and its subsequent association with perilipin on the lipid droplet surface. Based on previous studies linking PDE4D with adipose lipolysis, it was explored whether overexpression of PDE4D is sufficient to recapitulate the ability of FGF1 to suppress lipolysis (Jang et al., 2019). Indeed, overexpression of 3 PDE4D isoforms dose-dependently suppressed lipolysis in 3T3-L1 adipocytes, with the PDE4D3 isoform showing the highest efficacy (FIG. 9K). In order to extend on this finding, an adAAV vector was constructed that restricted PDE4D3 expression to mature adipocytes (O'Neill et al., 2014) (FIGS. 9L-9N). adAAV-PDE4D3-driven expression robustly suppressed isoproterenol-induced increases in lipolysis, cAMP, and perilipin-GFP/HSL-mCherry co-localization in 3T3-L1 adipocytes (FIGS. 3F-3H). In addition, lower pHSL levels were seen in 3T3-L1 adipocytes infected with adAAV-PDE4D3 compared to control adAAV-GFP (FIG. 9O). Furthermore, the reductions in isoproterenol-induced lipolysis and HSL phosphorylation with adAAV-PDE4D3 infection were conserved in human SVF-derived adipocytes (FIGS. 9P and 9Q).

### Example 5: FGF1-Induced Glucose Lowering is Dependent on PDE4 In Vivo

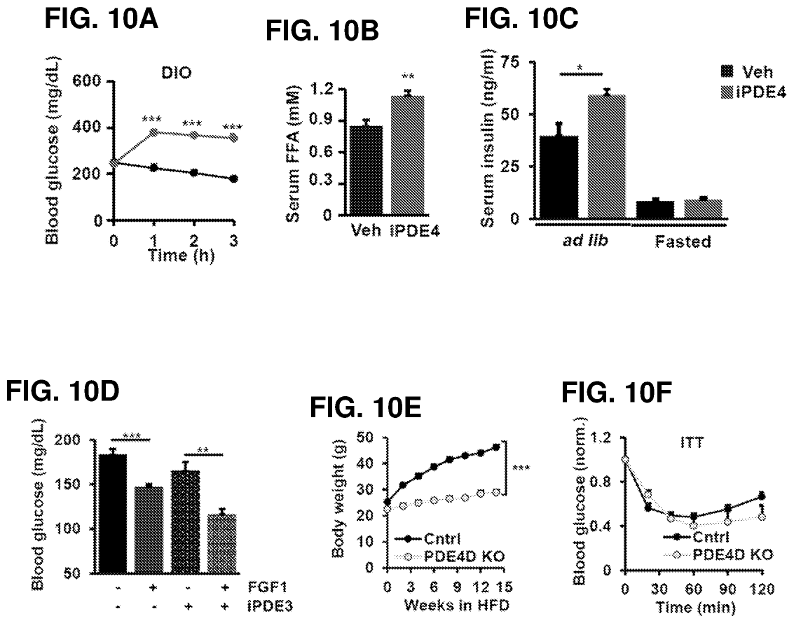

The finding that FGF1-induced suppression of lipolysis is dependent on PDE4 raised the possibility that this pathway contributes to glucose homeostasis in insulin-resistant mice. To explore this possibility, ad lib fed DIO mice were treated with the PDE4 inhibitor roflumilast. In these mice, PDE4 inhibition transiently increased blood glucose, serum FFA and insulin levels (FIGS. 4A and 4B, FIG. 10C). In fasted DIO mice, PDE4 inhibition led to a more sustained increase in blood glucose levels, presumably due to lower insulin levels and higher lipolysis in the fasted state (FIGS. 10A-10C). The ability of FGF1 to reduce blood glucose levels was lost with roflumilast pretreatment of ad lib fed mice (FIG. 4C). In contrast, inhibition of PDE3 failed to affect the ability of FGF1 to modulate glucose levels (FIG. 10D). These data support a requirement for PDE4-dependent regulation of lipolysis in the glucose lowering effects of FGF1. Given the ability of PDE4D to regulate lipolysis in vivo, experiments were performed to explore the role of this PDE family in the metabolic actions of FGF1. Adipose explants from PDE4D KO mice showed higher basal and isoproterenol-stimulated lipolysis, and adipocytes derived from PDE4D KO SVF were insensitive to FGF1 treatment (FIGS. 4D and 4E). In addition, despite lower body weight under HFD feeding, PDE4D KO mice developed insulin resistance comparable to controls (FIGS. 10E and 10F). In addition, FGF1 failed to lower blood glucose in these HFD-fed PDE4D KO mice; a defect that was restored with adAAV-driven expression of PDE4D3 in adipose tissue (FIGS. 4F and 4G). These data indicate that adipose PDE4D is required for the glucose lowering effects of exogenous FGF1.

### Example 6: FGF1-Induces Phosphorylation at a Regulatory Site on PDE4D3

The activities of phosphodiesterases are regulated by multiple phosphorylation events that integrate different signaling pathways (Mika and Conti, 2016). To explore whether FGF1 signaling induces PDE4D phosphorylation, isoproterenol-stimulated 3T3-L1 adipocytes were treated with FGF1. PDE4D was phosphorylated upon isoproterenol treatment, as evidenced by its decreased mobility in an SDS-PAGE gel (FIG. 5A). FGF1 co-treatment increased both the extent and duration of PDE4D phosphorylation (FIG. 5A). Consistent with this observation, an increase in PDE4D phosphorylation was seen in gWAT 30 minutes after FGF1 injection (FIG. 5B).

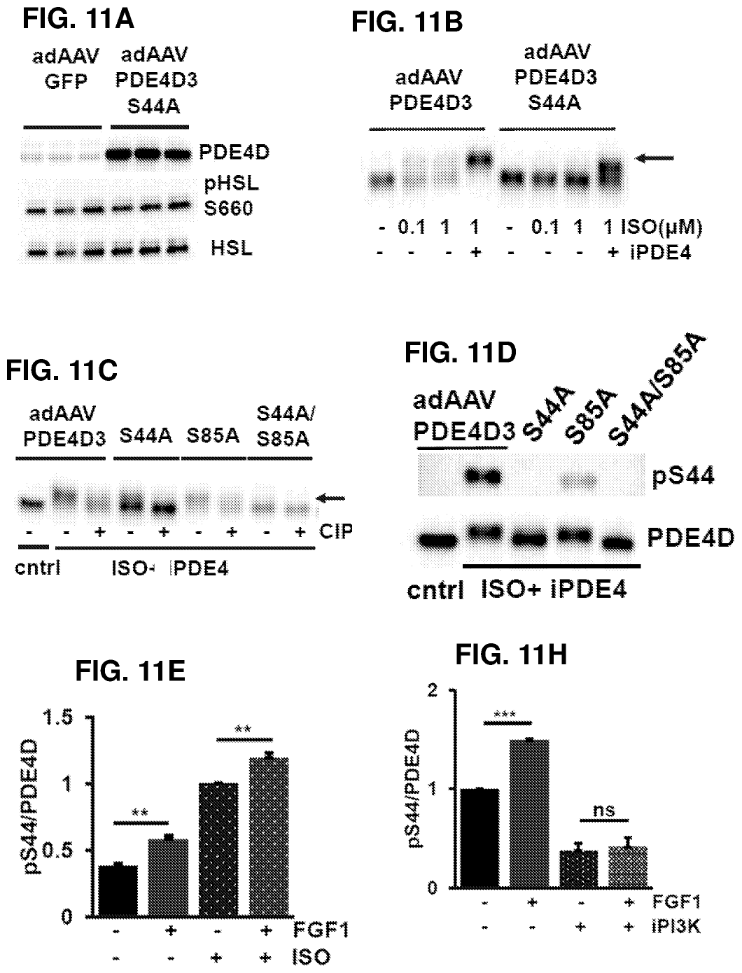

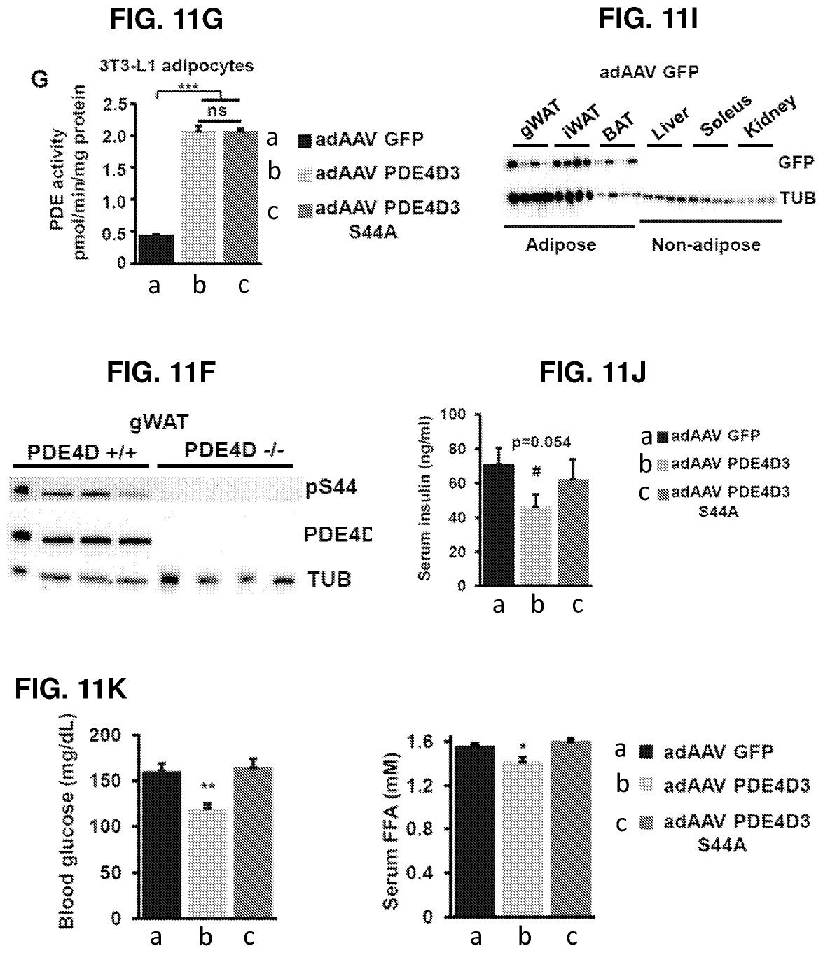

PDE4D proteins are phosphorylated by PKA at a conserved S85 site in the upstream conserved region 1 (S54 in humans and rats) that is thought to be necessary for activation, as well as at S44 (S13 in humans and rats), a PDE4D3-specific site in the N-terminus that does not affect PDE activity in vitro (FIG. 5C) (Hoffmann et al., 1998; Mika and Conti, 2016; Sette and Conti, 1996). To investigate the role of PDE4D3 phosphorylation in the regulation of lipolysis, adAAV expression constructs were generated in which these sites were mutated to alanine. Infection of 3T3-L1 adipocytes with a mutant incorporating S85 to alanine (S85A) largely replicated the ability of wildtype PDE4D3 to suppress lipolysis. In contrast, the S44A mutation abrogated the ability of PDE4D3 to affect lipolysis and HSL phosphorylation (FIG. 5D, FIG. 11A). Consistent with these findings, a reduced level of isoproterenol-induced phosphorylation was seen with the S44A mutant both in the absence and presence of PDE4 inhibition, implicating a regulatory role for S44 phosphorylation (FIG. 5E, FIG. 11B). Mutation of both phosphorylation sites (S44A S85A) further diminished the response to isoproterenol in the presence of a PDE4 inhibitor (treatment with calf intestinal phosphatase (CIP) confirmed that changes in electrophoretic mobility were due to phosphorylation; FIG. 5E, FIG. 11C). FGF1-induced phosphorylation of PDE4D3 was lost in the S44A mutant (FIG. 5F). Moreover, FGF1 treatment increased S44 phosphorylation in 3T3-L1 cells overexpressing PDE4D3 both in the absence and presence of isoproterenol, as determined using a polyclonal antibody that selectively recognizes PDE4D3 S44 phosphorylation (antibody specificity was confirmed in PDE4D KO gWAT; FIG. 5G, FIGS. 11E and 11F). WT PDE4D or S44A mutant showed similar in vitro PDE activity when overexpressed in adipocytes, indicating this site does not regulate in vitro catalytic activity agreeing with previous findings (FIG. 11G) (Carlisle Michel et al., 2004; Dodge et al., 2001). Consistent with a regulatory role of S44 phosphorylation, expression of PDE4D3 but not the S44A mutant restored the ability of FGF1 to suppress lipolysis in PDE4D KO SVF-derived adipocytes (FIG. 5H). Mechanistically, FGF1 induced S44 phosphorylation was inhibited by wortmannin in agreement with the dependence of FGF1 anti-lipolytic function on PI3K signaling (FIG. 5I, FIG. 11H).

The above data indicate that the specific phosphorylation of PDE4D at S44 is required for the anti-lipolytic activity of FGF1/PDE4D pathway. To confirm the in vivo relevance of this finding, ob/ob mice were injected with adAAV-GFP, adAAV-PDE4D3 or adAAV-PDE4D3 S44A (adipose tissue-specific expression was confirmed by Western blot; FIG. 11I). Overexpression of PDE4D3 from an adipocyte-specific promoter (human adiponectin promoter/enhancer) resulted in lower ad lib fed and overnight-fasted blood glucose and serum FFA levels, as well as a trend towards lower insulin levels in the fed state (FIGS. 5J and 5K, FIGS. 11J and 11K). In contrast, overexpression of the S44A mutant of PDE4D3 failed to affect these metabolic parameters. FGF1 induced a greater reduction in glucose levels in mice overexpressing PDE4D3, whereas the response in mice expressing the S44A mutant was indistinguishable from control mice (FIG. 5L).

FGF1 was identified as a fed-state adipokine whose expression is increased in response to high fat diet feeding (Jonker et al, 2012). In order to associate endogenous FGF1 signaling with S44 phosphorylation, gWAT depots were collected from chow and HFD fed mice under overnight fasted and refed conditions. Re-feeding approximately doubled the pS44 levels in both chow and HFD-fed mice. Additionally, HFD markedly reduced S44 phosphorylation in both the fasted and fed states, suggestive of a role for PDE4D in insulin-resistant hyperlipidemia (FIG. 5M). In combination, these findings support a mechanism in which exogenous FGF1 reduces serum glucose levels by suppressing adipose lipolysis in a PDE4D3-dependent manner, and implicates this mechanism in the physiological response to feeding.

### REFERENCES

Ahmad et al., (2009). Differential regulation of adipocyte PDE3B in distinct membrane compartments by insulin and the beta3-adrenergic receptor agonist CL316243: effects of caveolin-1 knockdown on formation/maintenance of macromolecular signalling complexes. Biochem J 424, 399-410. 10.1042/BJ20090842.
Azevedo et al., (2014). Clinical and molecular genetics of the phosphodiesterases (PDEs). Endocr Rev 35, 195-233. 10.1210/er.2013-1053.
Baeza-Raja et al., (2016). p75 Neurotrophin Receptor Regulates Energy Balance in Obesity. Cell Rep 14, 255-268. 10.1016/j.celrep.2015.12.028.
Bapat et al., (2015). Depletion of fat-resident Treg cells prevents age-associated insulin resistance. Nature 528, 137-141. 10.1038/nature 16151.
Bartness et al., (2014). Neural innervation of white adipose tissue and the control of lipolysis. Front Neuroendocrinol 35, 473-493. 10.1016/j.yfrne.2014.04.001.
Boden, G., Chen, X., and Stein, T. P. (2017). Gluconeogenesis in moderately and severely hyperglycemic patients with type 2 diabetes mellitus. American Journal of Physiology-Endocrinology and Metabolism. 10.1152/ajpendo.2001.280.1.e23.
Bunnell et al., (2008). Adipose-derived stem cells: isolation, expansion and differentiation. Methods 45, 115-120. 10.1016/j.ymeth.2008.03.006.
Carlisle Michel et al., (2004). PKA-phosphorylation of PDE4D3 facilitates recruitment of the mAKAP signalling complex. Biochemical Journal. 10.1042/BJ20040846.
Choi et al., (2016). Increased expression of FGF1-mediated signaling molecules in adipose tissue of obese mice. J Physiol Biochem 72, 157-167. 10.1007/s13105-016-0468-6.
Clifford et al., (2000). Translocation of hormone-sensitive lipase and perilipin upon lipolytic stimulation of rat adipocytes. Journal of Biological Chemistry. 10.1074/jbc.275.7.5011.
Degerman et al., (2011). From PDE3B to the regulation of energy homeostasis. Curr Opin Pharmacol 11, 676-682. 10.1016/j.coph.2011.09.015.
DiPilato et al., (2015a). The Role of PDE3B Phosphorylation in the Inhibition of Lipolysis by Insulin. Mol Cell Biol 35, 2752-2760. 10.1128/MCB.00422-15.
DiPilato et al., (2015b). The Role of PDE3B Phosphorylation in the Inhibition of Lipolysis by Insulin. Molecular and Cellular Biology. 10.1128/mcb.00422-15.
Dipilato et al., (2015). The Role of PDE3B Phosphorylation in the Inhibition of Lipolysis by Insulin. 35. 10.1128/MCB.00422-15.
Dodge, K. L., Khouangsathiene, S., Kapiloff, M.S., Mouton, R., Hill, E.V., Houslay, M.D., Langeberg, L. K., and Scott, J.D. (2001). mAKAP assembles a protein kinase A/PDE4 phosphodiesterase cAMP signaling module. EMBO Journal. 10.1093/emboj/20.8.1921.
Duncan, R.E., Ahmadian, M., Jaworski, K., Sarkadi-Nagy, E., and Sul, H.S. (2007). Regulation of lipolysis in adipocytes. Annu Rev Nutr 27, 79-101. 10.1146/annurev.nutr.27.061406.093734.
Egan, J.J., Greenberg, A.S., Chang, M.K., Wek, S.A., Moos, M. C., and Londos, C. (1992). Mechanism of hormone-stimulated lipolysis in adipocytes: translocation of hormone-sensitive lipase to the lipid storage droplet. Proceedings of the National Academy of Sciences. 10.1073/pnas.89.18.8537.
Exton, J. H., and Park, C.R. (1967). Control of gluconeogenesis in liver. I. General features of gluconeogenesis in the perfused livers of rats. Journal of Biological Chemistry.
Funicello, M., Novelli, M., Ragni, M., Vottari, T., Cocuzza, C., Soriano-Lopez, J., Chiellini, C., Boschi, F., Marzola, P., Masiello, P., et al. (2007). Cathepsin K null mice show reduced adiposity during the rapid accumulation of fat stores. PLOS One 2, e683. 10.1371/journal.pone.0000683.
Grange, M., Sette, C., Cuomo, M., Conti, M., Lagarde, M., Prigent, A.-F., and Némoz, G. (2000). The CAMP-specific Phosphodiesterase PDE4D3 Is Regulated by Phosphatidic Acid Binding. Journal of Biological Chemistry. 10.1074/jbc.m006329200.
Greenberg, A.S., Egan, J.J., Wek, S.A., Garty, N.B., Blanchette-Mackie, E. J., and Londos, C. (1991). Perilipin, a major hormonally regulated adipocyte-specific phosphoprotein associated with the periphery of lipid storage droplets. Journal of Biological Chemistry.
Grønning, et al., (2006). Reduced PDE4 expression and activity contributes to enhanced catecholamine-induced cAMP accumulation in adipocytes from FOXC2 transgenic mice. FEBS Lett 580, 4126-4130. 10.1016/j.febslet.2006.06.058.
Hoffmann et al., (1998). cAMP-specific phosphodiesterase HSPDE4D3 mutants which mimic activation and changes in rolipram inhibition triggered by protein kinase A phosphorylation of Ser-54: generation of a molecular model. Biochem J 333 (Pt 1), 139-149. 10.1042/bj3330139.
Houslay, M.D., and Adams, D.R. (2003). PDE4 cAMP phosphodiesterases: modular enzymes that orchestrate signalling cross-talk, desensitization and compartmentalization. Biochem J 370, 1-18. 10.1042/BJ20021698.
Inagaki et al., (2007). Endocrine regulation of the fasting response by PPARalpha-mediated induction of fibroblast growth factor 21. Cell Metab 5, 415-425. 10.1016/j.cmet.2007.05.003.
Jang et al., (2020). Theobromine alleviates diet-induced obesity in mice via phosphodiesterase-4 inhibition. Eur J Nutr 59, 3503-3516. 10.1007/s00394-020-02184-6.
Jin, S. L., and Conti, M. (2002). Induction of the cyclic nucleotide phosphodiesterase PDE4B is essential for LPS-activated TNF-alpha responses. Proc Natl Acad Sci USA 99, 7628-7633. 10.1073/pnas. 122041599.
Jin, S.L., Lan, L., Zoudilova, M., and Conti, M. (2005). Specific role of phosphodiesterase 4B in lipopolysaccharide-induced signaling in mouse macrophages. J Immunol 175, 1523-1531. 10.4049/jimmunol. 175.3.1523.
Jin, S.L., Richard, F.J., Kuo, W.P., D'Ercole, A. J., and Conti, M. (1999). Impaired growth and fertility of CAMP-specific phosphodiesterase PDE4D-deficient mice. Proc Natl Acad Sci USA 96, 11998-12003. 10.1073/pnas.96.21.11998.
Jonker et al., (2012). A PPARγ-FGF1 axis is required for adaptive adipose remodelling and metabolic homeostasis. Nature 485, 391-394. 10.1038/nature 10998.
Kitamura et al., (1999). Insulin-induced phosphorylation and activation of cyclic nucleotide phosphodiesterase 3B by the serine-threonine kinase Akt. Mol Cell Biol 19, 6286-6296. 10.1128/mcb.19.9.6286.
Komatsu et al., (2013). Inhibition of PDE4B suppresses inflammation by increasing expression of the deubiquitinase CYLD. Nat Commun 4, 1684. 10.1038/ncomms2674.
Kraynik, S.M., Miyaoka, R. S., and Beavo, J.A. (2013). PDE3 and PDE4 Isozyme-Selective Inhibitors Are Both Required for Synergistic Activation of Brown Adipose Tissue s. 4, 1155-1165.
Lin, H. V., and Accili, D. (2011). Hormonal regulation of hepatic glucose production in health and disease.
Lombardo, Y. B., and Menahan, L.A. (1979). Gluconeogenesis in perfused livers of genetically obese-hyperglycemic (ob/ob) mice. Hormone and Metabolic Research. 10.1055/s-0028-1092672.
Mackenzie, S.J., Baillie, G.S., McPhee, I., Mackenzie, C., Seamons, R., McSorley, T., Millen, J., Beard, M.B., van Hecke, G., and Houslay, M.D. (2002). Long PDE4 cAMP specific phosphodiesterases are activated by protein kinase A-mediated phosphorylation of a single serine residue in Upstream Conserved Region 1 (UCR1). Br J Pharmacol 136, 421-433. 10.1038/sj.bjp.0704743.
Mika, D., and Conti, M. (2016). PDE4D phosphorylation: A coincidence detector integrating multiple signaling pathways.
Möllmann et al., (2017). The PDE4 inhibitor roflumilast reduces weight gain by increasing energy expenditure and leads to improved glucose metabolism. Diabetes Obes Metab 19, 496-508. 10.1111/dom.12839.
Nakamura et al., (2004). Augmentation of lipolysis in adipocytes from fed rats, but not from starved rats, by inhibition of rolipram-sensitive phosphodiesterase 4. 425, 106-114. 10.1016/j.abb.2004.02.036.
O'Neill et al., (2014). Targeting adipose tissue via systemic gene therapy. Gene Therapy. 10.1038/gt.2014.38.
Oki et al., (2000). Short term feedback regulation of cAMP in FRTL-5 thyroid cells. Role of PDE4D3 phosphodiesterase activation. J Biol Chem 275, 10831-10837. 10.1074/jbc.275.15.10831.
Oknianska et al., (2007). Long-term regulation of cyclic nucleotide phosphodiesterase type 3B and 4 in 3T3-L1 adipocytes. Biochem Biophys Res Commun 353, 1080-1085. 10.1016/j.bbrc.2006.12.141.
Ong et al., (2009). The role of the PDE4D cAMP phosphodiesterase in the regulation of glucagon-like peptide-1 release. British Journal of Pharmacology. 10.1111/j.1476-5381.2009.00194.x.
Park, S.J., Ahmad, F., Philp, A., Baar, K., Williams, T., Luo, H., Ke, H., Rehmann, H., Taussig, R., Brown, A.L., et al. (2012). Resveratrol ameliorates aging-related metabolic phenotypes by inhibiting cAMP phosphodiesterases. Cell. 10.1016/j.cell.2012.01.017.
Payne, J., and Morris, J.G. (1969). Pyruvate carboxylase in Rhodopseudomonas spheroides. J Gen Microbiol 59, 97-101. 10.1099/00221287-59-1-97.
Perry et al., (2015a). Hepatic acetyl COA links adipose tissue inflammation to hepatic insulin resistance and type 2 diabetes. Cell 160, 745-758. 10.1016/j.cell.2015.01.012.
Perry et al., (2015b). FGF1 and FGF19 reverse diabetes by suppression of the hypothalamic-pituitary-adrenal axis. Nat Commun 6, 6980. 10.1038/ncomms7980.
Perry et al., (2018). Leptin Mediates a Glucose-Fatty Acid Cycle to Maintain Glucose Homeostasis in Starvation. Cell 172, 234-248.e217. 10.1016/j.cell.2017.12.001.
Qiao et al., (2011). Liver-specific microRNA-122 target sequences incorporated in AAV vectors efficiently inhibits transgene expression in the liver. Gene Therapy. 10.1038/gt.2010.157.
Rahn et al., (1994). Essential role of phosphatidylinositol 3-kinase in insulin-induced activation and phosphorylation of the cGMP-inhibited CAMP phosphodiesterase in rat adipocytes. Studies using the selective inhibitor wortmannin. FEBS Lett 350, 314-318. 10.1016/0014-5793 (94) 00797-7.
Rebrin et al., (1996). Causal linkage between insulin suppression of lipolysis and suppression of liver glucose output in dogs. J Clin Invest 98, 741-749. 10.1172/JCI118846.
Rybalkin et al., (2013). Enzyme assays for cGMP hydrolyzing phosphodiesterases. Methods Mol Biol 1020, 51-62. 10.1007/978-1-62703-459-3_3.
Saponaro et al., (2015). The Subtle Balance between Lipolysis and Lipogenesis: A Critical Point in Metabolic Homeostasis. Nutrients 7, 9453-9474. 10.3390/nu7115475.
Scarlett et al., (2019). Peripheral Mechanisms Mediating the Sustained Antidiabetic Action of FGF1 in the Brain. Diabetes 68, 654-664. 10.2337/db18-0498.
Scarlett et al., (2016). Central injection of fibroblast growth factor I induces sustained remission of diabetic hyperglycemia in rodents. Nat Med 22, 800-806. 10.1038/nm.4101.
Sears, B., and Perry, M. (2015). The role of fatty acids in insulin resistance. Lipids Health Dis 14, 121. 10.1186/s12944-015-0123-1.
Sette, C., and Conti, M. (1996). Phosphorylation and activation of a cAMP-specific phosphodiesterase by the cAMP-dependent protein kinase. Involvement of serine 54 in the enzyme activation. J Biol Chem 271, 16526-16534. 10.1074/jbc.271.28.16526.
Shen et al., (2009). Functional interaction of hormone-sensitive lipase and perilipin in lipolysis. Journal of Lipid Research. 10.1194/jlr.m900176-jlr200.
Shrago, E., and Lardy, H.A. (1966). Paths of carbon in gluconeogenesis and lipogenesis. II. Conversion of precursors to phosphoenolpyruvate in liver cytosol. Journal of Biological Chemistry.
Snyder et al., (2005). The role of cyclic nucleotide phosphodiesterases in the regulation of adipocyte lipolysis. 46. 10.1194/jlr.M400362-JLR200.
Strålfors, P., and Honnor, R.C. (1989). Insulin-induced dephosphorylation of hormone-sensitive lipase. Correlation with lipolysis and cAMP-dependent protein kinase activity. Eur J Biochem 182, 379-385. 10.1111/j.1432-1033.1989.tb14842.x.
Suh et al., (2014). Endocrinization of FGF1 produces a neomorphic and potent insulin sensitizer. Nature 513, 436-439. 10.1038/nature 13540.
Tang et al., (2001). Phosphodiesterase 3B gene expression is enhanced in the liver but reduced in the adipose tissue of obese insulin resistant db/db mouse. Diabetes Res Clin Pract 54, 145-155. 10.1016/s0168-8227 (01) 00271-6.
Tewson et al., (2016). New DAG and cAMP Sensors Optimized for Live-Cell Assays in Automated Laboratories. J Biomol Screen 21, 298-305. 10.1177/1087057115618608.
Turner et al., (2005). Sources of plasma glucose and liver glycogen in fasted ob/ob mice. Acta Diabetologica. 10.1007/s00592-005-0201-3.
van Dijk, T.H., Boer, T.S., Havinga, R., Stellaard, F., Kuipers, F., and Reijngoud, D.J. (2003). Quantification of hepatic carbohydrate metabolism in conscious mice using serial blood and urine spots. Anal Biochem 322, 1-13. 10.1016/j.ab.2003.07.008.
Vollert et al., (2012). The glucose-lowering effects of the PDE4 inhibitors roflumilast and roflumilast-N-oxide in db/db mice. Diabetologia. 10.1007/s00125-012-2632-z.
Wang et al., (2020). Adipocyte Piezol mediates obesogenic adipogenesis through the FGF1/FGFR1 signaling pathway in mice. Nat Commun 11, 2303. 10.1038/s41467-020-16026-w.
Young et al., (2006). Alterations in regulation of energy homeostasis in cyclic nucleotide phosphodiesterase 3B-null mice. Journal of Clinical Investigation. 10.1172/JCI24867.
Yuan et al., (2012). A positive/negative ion-switching, targeted mass spectrometry-based metabolomics platform for bodily fluids, cells, and fresh and fixed tissue. Nat Protoc 7, 872-881. 10.1038/nprot.2012.024.
Zhang et al., (2009). Reduced adiposity and high-fat diet-induced adipose inflammation in mice deficient for phosphodiesterase 4B. Endocrinology 150, 3076-3082. 10.1210/en.2009-0108.

In view of the many possible aspects to which the principles of the disclosed subject matter may be applied, it should be recognized that the illustrated aspects are only examples of the disclosure and should not be taken as limiting the scope of the disclosure. Rather, the scope of the disclosure is defined by the following claims. We therefore claim all that comes within the scope and spirit of these claims.

1. (canceled)

2. A method of treating type 2 diabetes or reducing blood glucose in a subject, comprising:
administering to the subject a therapeutically effective amount of an agent that increases expression or activity of phosphodiesterase 4D isoform 3 (PDE4D3) in adipocytes of the subject, thereby treating the type 2 diabetes or reducing blood glucose.

3. The method of claim 2, wherein the agent that increases expression or activity of PDE4D3 comprises a nucleic acid molecule encoding a PDE4D3 protein.

4. The method of claim 3, wherein the nucleic acid molecule encoding the PDE4D3 protein is operably linked to an adipocyte-specific promoter, and/or
wherein the nucleic acid molecule encoding the PDE4D3 protein comprises a vector.

5. (canceled)

6. The method claim 4, wherein the vector is a viral vector.

7. The method of claim 6, wherein the viral vector is an adenovirus vector or an adeno-associated virus (AAV) vector.

8. The method of claim 3, wherein the nucleic acid molecule encoding the PDE4D3 protein is introduced into adipocytes using a gene editing method.

9. The method of claim 8, wherein the gene editing method comprises clustered regularly interspaced short palindromic repeats (CRISPR)-CRISPR-associated protein 9 (Cas9), transcription activator-like effector nucleases (TALENs), or zinc-finger nucleases (ZFNs).

10. The method of claim 3, wherein the PDE4D3 protein comprises the amino acid sequence set forth as SEQ ID NO: 1.

11. The method of claim 2, wherein the agent that increases expression or activity of PDE4D3 is a small molecule activator of PDE4D3.

12. The method of claim 11, wherein the small molecule activator of PDE4D3 is
an N-substituted-2-(3-aryl-1H-1,2,4-triazol-1-yl) acetamide,
2-(3-(4-chloro-3-fluorophenyl)-5-ethyl-1H-1,2,4-triazol-1-yl)-N-(3,5-dichlorobenzyl)acetamide (MR-L2),
conjugated to an antibody that specifically binds fibroblast growth factor receptor 1b (FGFR1b), or
conjugated to an incretin.

13.-15. (canceled)

16. The method of claim 12, wherein the incretin is gastric inhibitory peptide (GIP) or glucagon-like peptide-1 (GLP-1) or
wherein the small molecule activator of PDE4D is conjugated to the antibody or the incretin via an acid-sensitive linker.

17. (canceled)

18. The method of claim 2, further comprising administering to the subject a therapeutically effective amount of a mature fibroblast growth factor 1 (FGF1) protein or a modified mature FGF1 protein.

19. The method of claim 18, wherein the modified mature FGF1 protein
has reduced mitogenicity and/or increased stability compared to native FGF1 protein, or
selectively binds FGFR1b.

20. (canceled)

21. The method of claim 2, further comprising administering a therapeutically effective amount of an additional therapeutic compound.

22. The method of claim 21, wherein the additional therapeutic compound is insulin, an alpha-glucosidase inhibitor, amylin agonist, dipeptidyl-peptidase 4 (DPP-4) inhibitor, meglitinide, sulfonylurea, or a peroxisome proliferator-activated receptor (PPAR)-gamma agonist.

23. The method of claim 22, wherein the PPAR-gamma agonist is a thiazolidinedione (TZD), aleglitazar, farglitazar, muraglitazar, or tesaglitazar.

24. The method of claim 23, wherein the TZD is pioglitazone, rosiglitazone, rivoglitazone, or troglitazone.

25. (canceled)

26. A nucleic acid molecule comprising a phosphodiesterase 4D isoform 3 (PDE4D3) coding sequence operably linked to an adipocyte-specific promoter.

27. A vector comprising the nucleic acid molecule of claim 26.

28. The vector of claim 27, which is a viral vector or a plasmid vector.

29. (canceled)

30. A composition comprising a small molecule activator of phosphodiesterase 4D isoform 3 (PDE4D3) conjugated to:
an antibody that specifically binds fibroblast growth factor receptor 1b (FGFR1b); or
an incretin.

31. The composition of claim 30, wherein the incretin is gastric inhibitory peptide (GIP) or glucagon-like peptide-1 (GLP-1), or
wherein the small molecule activator of PDE4D3 is 2-(3-(4-chloro-3-fluorophenyl)-5-ethyl-1H-1,2,4 triazol-1-yl)-N-(3,5-dichlorobenzyl)acetamide (MR-L2).

32. (canceled)

33. A pharmaceutical composition, comprising a pharmaceutically acceptable carrier and the nucleic acid molecule of claim 26.


SEQUENCE LISTING (37 sequences)

SEQ ID NO: 1 - DNA, 7777 bp, Homo sapiens
  source 1..7777: mol_type = genomic DNA
ggattcaatacttgttgcaataattgcccacgatagctgctcaaacaagagagttggaattcatctgtaaaaatcactacatgtaacgtaggagacaagaaaaatattaatgacagaagatctgcgaacatgatgcacgtgaataattttccctttagaaggcattcctggatatgttttgatgtggacaatggcacatctgcgggacggagtcccttggatcccatgaccagcccaggatccgggctaattctccaagcaaattttgtccacagtcaacgacgggagtccttcctgtatcgatccgacagcgattatgacctctctccaaagtctatgtcccggaactcctccattgccagtgatatacacggagatgacttgattgtgactccatttgctcaggtcttggccagtctgcgaactgtacgaaacaactttgctgcattaactaatttgcaagatcgagcacctagcaaaagatcacccatgtgcaaccaaccatccatcaacaaagccaccataacagaggaggcctaccagaaactggccagcgagaccctggaggagctggactggtgtctggaccagctagagaccctacagaccaggcactccgtcagtgagatggcctccaacaagtttaaaaggatgcttaatcgggagctcacccatctctctgaaatgagtcggtctggaaatcaagtgtcagagtttatatcaaacacattcttagataagcaacatgaagtggaaattccttctccaactcagaaggaaaaggagaaaaagaaaagaccaatgtctcagatcagtggagtcaagaaattgatgcacagctctagtctgactaattcaagtatcccaaggtttggagttaaaactgaacaagaagatgtccttgccaaggaactagaagatgtgaacaaatggggtcttcatgttttcagaatagcagagttgtctggtaaccggcccttgactgttatcatgcacaccatttttcaggaacgggatttattaaaaacatttaaaattccagtagatactttaattacatatcttatgactctcgaagaccattaccatgctgatgtggcctatcacaacaatatccatgctgcagatgttgtccagtctactcatgtgctattatctacacctgctttggaggctgtgtttacagatttggagattcttgcagcaatttttgccagtgcaatacatgatgtagatcatcctggtgtgtccaatcaatttctgatcaatacaaactctgaacttgccttgatgtacaatgattcctcagtcttagagaaccatcatttggctgtgggctttaaattgcttcaggaagaaaactgtgacattttccagaatttgaccaaaaaacaaagacaatctttaaggaaaatggtcattgacatcgtacttgcaacagatatgtcaaaacacatgaatctactggctgatttgaagactatggttgaaactaagaaagtgacaagctctggagttcttcttcttgataattattccgataggattcaggttcttcagaatatggtgcactgtgcagatctgagcaacccaacaaagcctctccagctgtaccgccagtggacggaccggataatggaggagttcttccgccaaggagaccgagagagggaacgtggcatggagataagccccatgtgtgacaagcacaatgcttccgtggaaaaatcacaggtgggcttcatagactatattgttcatcccctctgggagacatgggcagacctcgtccaccctgacgcccaggatattttggacactttggaggacaatcgtgaatggtaccagagcacaatccctcagagcccctctcctgcacctgatgacccagaggagggccggcagggtcaaactgagaaattccagtttgaactaactttagaggaagatggtgagtcagacacggaaaaggacagtggcagtcaagtggaagaagacactagctgcagtgactccaagactctttgtactcaagactcagagtctactgaaattccccttgatgaacaggttgaagaggaggcagtaggggaagaagaggaaagccagcctgaagcctgtgtcatagatgatcgttctcctgacacgtaacagtgcaaaaactttcatgccttttttttttttaagtagaaaaattgtttccaaagtgcatgtcacatgccacaaccacggtcacacctcactgtcatctgccaggacgtttgttgaacaaaactgaccttgactactcagtccagcgctcaggaatatcgtaaccagttttttcacctccatgtcatccgagcaaggtggacatcttcacgaacagcgtttttaacaagatttcagcttggtagagctgacaaagcagataaaatctactccaaattattttcaagagagtgtgactcatcaggcagcccaaaagtttattggacttggggtttctattcctttttatttgtttgcaatattttcagaagaaaggcattgcacagagtgaacttaatggacgaagcaacaaatatgtcaagaacaggacatagcacgaatctgttaccagtaggaggaggatgagccacagaaattgcataattttctaatttcaagtcttcctgatacatgactgaatagtgtggttcagtgagctgcactgacctctacattttgtatgatatgtaaaacagattttttgtagagcttacttttattattaaatgtattgaggtattatatttaaaaaaaactatgttcagaacttcatctgccactggttatttttttctaaggagtaacttgcaagttttcagtacaaatctgtgctacactggataaaaatctaatttatgaattttacttgcaccttatagttcatagcaattaactgatttgtagtgattcattgtttgttttatataccaatgacttccatattttaaaagagaaaaacaactttatgttgcaggaaaccctttttgtaagtctttattatttactttgcattttgtttcactctttccagataagcagagttgctcttcaccagtgtttttcttcatgtgcaaagtgactatttgttctataatacttttatgtgtgttatatcaaatgtgtcttaagcttcatgcaaactcagtcatcagttcgtgttgtctgaagcaagtgggagatatataaatacccagtagctaaaatggtcagtcttttttagatgttttcctacttagtatctcctaataacgttttgctgtgtcactagatgttcatttcacaagtgcatgtctttctaataatccacacatttcatgctctaataatccacacatttcatgctcatttttattgtttttacagccagttatagtaagaaaaaggtttttccccttgtgctgctttataatttagcgtgtgtctgaaccttatccatgtttgctagatgaggtcttgtcaaatatatcactaccattgtcaccggtgaaaagaaacaggtagttaagttagggttaacattcatttcaaccacgaggttgtatatcatgactagcttttactcttggtttacagagaaaagttaaacagccaactaggcagtttttaagaatattaacaatatattaacaaacaccaatacaactaatcctatttggttttaatgatttcaccatgggattaagaactatatcaggaacatccctgagaaacggttttaagtgtagcaactactcttccttaatggacagccacataacgtgtaggaagtcctttatcacttatcctcgatccataagcatatcttgcagaggggaactacttctttaaacacatggagggaaagaagatgatgccactggcaccagagggttagtactgtgatgcatcctaaaatatttattatattggtaaaaattctggttaaataaaaaattagagatcactcttggctgatttcagcaccaggaactgtattacagttttagagattaattcctagtgtttacctgattatagcagttggcatcatggggcatttaattctgactttatccccacgtcagccttaataaagtcttctttaccttctctatgaagactttaaagcccaaataatcatttttcacattgatattcaagaattgagatagatagaagccaaagtgggtatctgacaagtggaaaatcaaacgtttaagaagaattacaactctgaaaagcatttatatgtggaacttctcaaggagcctcctggggactggaaagtaagtcatcagccaggcaaatgactcatgctgaagagagtccccatttcagtcccctgagatctagctgatgcttagatcctttgaaataaaaattatgtctttataactctgatcttttacataaagcagaagaggaatcaactagttaattgcaaggtttctactctgtttcctctgtaaagatcagatggtaatctttcaaataagaaaaaaataaagacgtatgtttgaccaagtagtttcacaagaatatttgggaacttgtttcttttaattttatttgtccctgagtgaagtctagaaagaaaggtaaagagtctagagtttattcctctttccaaaacattctcattcctctcctccctacacttagtatttcccccacagagtgcctagaatcttaataatgaataaaataaaaagcagcaatatgtcattaacaaatccagacctgaaagggtaaagggtttataactgcactaataaagagaggctctttttttttcttccagtttgttggtttttaatggtaccgtgttgtaaagatacccactaatggacaatcaaattgcagaaaaggctcaatatccaagagacagggactaatgcactgtacaatctgcttatccttgcccttctctcttgccaaagtgtgcttcagaaatatatactgctttaaaaaagaataaaagaatatccttttacaagtggctttacatttcctaaaatgccataagaaaatgcaatatctgggtactgtatggggaaaaaaatgtccaagtttgtgtaaaaccagtgcatttcagcttgcaagttactgaacacaataatgctgttttaattttgttttatatcagttaaaattcacaataatgtagatagaacaaattacagacaaggaaagaaaaaacttgaatgaaatggattttacagaaagctttatgataatttttgaatgcattatttattttttgtgccatgcattttttttctcaccaaatgaccttacctgtaatacagtcttgtttgtctgtttacaaccatgtatttattgcaatgtacatactgtaatgttaattgtaaattatctgttcttattaaaacatcatcccatgatgggatggtgttgatatatttggaaactcttggtgagagaatgaatggtgtgtatacatactctgtacatttttcttttctcctgtaatatagtcttgtcaccttagagcttgtttatggaagattcaagaaaactataaaatacttaaagatatataaatttaaaaaaacatagctgcaggtctttggtcccagggctgtgccttaactttaaccaatattttcttctgttttgctgcatttgaaaggtaacagtggagctagggctgggcattttacatccaggcttttaattgattagaattctgccaataggtggattttacaaaaccacagacaacctctgaaagattctgagacccttttgagacagaagctcttaagtacttcttgccagggagcagcactgcatgtgtgatggttgtttgccatctgttgatcaggaactacttcagctacttgcatttgattatttcctttttttttttttttaactcggaaacacaactggggaaatatattctttcccagtgattataaacaatctttttcttttttttaagtccttttggcttctagagctcataggaaaatggacttgatttgaaattggagccagagtttactcgtgttggttatctattcatcagcttcctgacatgttaagagaatacattaaagagaaaatactgttttttaatcctaaaatttttcttccactaagataaaccaaatgtccttacatatatgtaaacccatctatttaaacgcaaaggtgggttgatgtcagtttacatagcagaaagcattcactatcctctaagatttgtttctgcaaaactttcattgctttagaattttaaaatttcaccttgtacaatggccagcccctaaagcaggaaacatttataatggattatatggaaacatcctcccagtacttgcccagcccttgaatcatgtggcttttcagtgaaaggaaagattctttttctaggaaaaatgagcctattttattttattttattttattttttgacacaaactgtagattttagcagccctggcccaaaggaatttgattacttttgttttaaacagtacaaaggggacactataattacaaaaacatccttaactgatttgagttgtttttatttctttggatatattttcagagtggtaaattgtgtgtgagaattacaaatgattattcttttagtggtttcttagcctctcttacagcccacggggatagtactgtacatcaataccttcatatgaaatttttatatgcaatgaaaataaaagcatgggttgattctgcctatttatgactcaatcttttacaaataaaagattattcattttaaattatagttcaatcagcatgtctcttaggatactgaacgtggttgaaatgaaaggatagtgacatcataagttagtactgatattcataaccaaataaagccaacttgagtaattttgctacattaaaaattaccaaaattacttagatggcctataagattaagcatggtgttttctaagcaagctttgaaaggggccttccatacttacttaattgaatattctgggatattgaaaattattcagatacttgacaattatttttggttacctactccgcaaactacaaagttttaaggactcaacaataagttaatgagacacagtgtttgctttcatggagcttacagtctggaggggacaaaggcttaaacaatactcatataattatatatgtgatcagtacaatgaaggagctcagtggggtaaataagcaggaacctgaacttgatctgttccggagggccacagaaggcttccttgaggccttgagaaagtgatttgcatctgagttctgaaggattgtaagaggtaactagggaaaaagttgacaggaagaggaaggggatccagacaagaaacatttgcaaagatcttgaggcataaatgagcttgagacatctggagaaactgaggaaaagtgagagagtaggcagggcctggagccgcagagccattgctaaccatcctgtgtgagatatcccccattctgtagctttattctcataaccctgctcaattttctttataacacttctcacagatttatatacgtgtttgtttttgttatctgtctctcccaccagaccacagctccatgagagcaaggtctttgcttaccaatatatcactagcacttaaaactatgcctggtacacagtaggttcttaatatgtgttgaatatagccatcaaattgatattggatataattcaatctgataagatattttgagatattaaagagtttttaacttgataccataaaaa

SEQ ID NO: 2 - protein, 673 aa, Homo sapiens
MMHVNNFPFRRHSWICFDVDNGTSAGRSPLDPMTSPGSGLILQANFVHSQRRESFLYRSDSDYDLSPKSMSRNSSIASDIHGDDLIVTPFAQVLASLRTVRNNFAALTNLQDRAPSKRSPMCNQPSINKATITEEAYQKLASETLEELDWCLDQLETLQTRHSVSEMASNKFKRMLNRELTHLSEMSRSGNQVSEFISNTFLDKQHEVEIPSPTQKEKEKKKRPMSQISGVKKLMHSSSLTNSSIPRFGVKTEQEDVLAKELEDVNKWGLHVFRIAELSGNRPLTVIMHTIFQERDLLKTFKIPVDTLITYLMTLEDHYHADVAYHNNIHAADVVQSTHVLLSTPALEAVFTDLEILAAIFASAIHDVDHPGVSNQFLINTNSELALMYNDSSVLENHHLAVGFKLLQEENCDIFQNLTKKQRQSLRKMVIDIVLATDMSKHMNLLADLKTMVETKKVTSSGVLLLDNYSDRIQVLQNMVHCADLSNPTKPLQLYRQWTDRIMEEFFRQGDRERERGMEISPMCDKHNASVEKSQVGFIDYIVHPLWETWADLVHPDAQDILDTLEDNREWYQSTIPQSPSPAPDDPEEGRQGQTEKFQFELTLEEDGESDTEKDSGSQVEEDTSCSDSKTLCTQDSESTEIPLDEQVEEEAVGEEEESQPEACVIDDRSPDT

SEQ ID NO: 3 - DNA, 27 bp, synthetic construct
  misc_feature 1..27: note = oligonucleotide primer
  source 1..27: mol_type = other DNA
caccatgctcaaaccagagtgttggga

SEQ ID NO: 4 - DNA, 25 bp, synthetic construct
  misc_feature 1..25: note = oligonucleotide primer
  source 1..25: mol_type = other DNA
caccatggcttttgtctgggatccc

SEQ ID NO: 5 - DNA, 31 bp, synthetic construct
  misc_feature 1..31: note = oligonucleotide primer
  source 1..31: mol_type = other DNA
caccatgagcattataatgaagccaagatcc

SEQ ID NO: 6 - DNA, 24 bp, synthetic construct
  misc_feature 1..24: note = oligonucleotide primer
  source 1..24: mol_type = other DNA
ttacgtatcaggacagcagtcatc

SEQ ID NO: 7 - DNA, 59 bp, synthetic construct
  misc_feature 1..59: note = oligonucleotide primer
  source 1..59: mol_type = other DNA
aaagatatccaagtttgtacaaaaaagcaggctgccaccatgctcaaaccagagtgttg

SEQ ID NO: 8 - DNA, 30 bp, synthetic construct
  misc_feature 1..30: note = oligonucleotide primer
  source 1..30: mol_type = other DNA
aaacatatgttacgtatcaggacagcagtc

SEQ ID NO: 9 - DNA, 59 bp, synthetic construct
  misc_feature 1..59: note = oligonucleotide primer
  source 1..59: mol_type = other DNA
aaagatatccaagtttgtacaaaaaagcaggctgccaccatggtgagcaagggcgagga

SEQ ID NO: 10 - DNA, 31 bp, synthetic construct
  misc_feature 1..31: note = oligonucleotide primer
  source 1..31: mol_type = other DNA
aaacatatgttacttgtacagctcgtccatg

SEQ ID NO: 11 - DNA, 69 bp, synthetic construct
  misc_feature 1..69: note = oligonucleotide primer
  source 1..69: mol_type = other DNA
aaagatatcactagtcaagtttgtacaaaaaagcaggctgccaccatggatttacgcacgatgacacag

SEQ ID NO: 12 - DNA, 29 bp, synthetic construct
  misc_feature 1..29: note = oligonucleotide primer
  source 1..29: mol_type = other DNA
aaaacgcgtgttcagtggtgcagcaggcg

SEQ ID NO: 13 - DNA, 46 bp, synthetic construct
  misc_feature 1..46: note = oligonucleotide primer
  source 1..46: mol_type = other DNA
aaagatatcactagtacgcgtggatccatggtgagcaagggcgagg

SEQ ID NO: 14 - DNA, 31 bp, synthetic construct
  misc_feature 1..31: note = oligonucleotide primer
  source 1..31: mol_type = other DNA
aaacatatgtcacttgtacagctcgtccatg

SEQ ID NO: 15 - DNA, 67 bp, synthetic construct
  misc_feature 1..67: note = oligonucleotide primer
  source 1..67: mol_type = other DNA
aaagatatcactagtcaagtttgtacaaaaaagcaggctgccaccatgtcaatgaacaagggcccaa

SEQ ID NO: 16 - DNA, 32 bp, synthetic construct
  misc_feature 1..32: note = oligonucleotide primer
  source 1..32: mol_type = other DNA
aaacatatgttaaactctttcttcaccggcat

SEQ ID NO: 17 - DNA, 36 bp, synthetic construct
  misc_feature 1..36: note = oligonucleotide primer
  source 1..36: mol_type = other DNA
cttccctttcagaaggcatgcctggatatgtttcga

SEQ ID NO: 18 - DNA, 36 bp, synthetic construct
  misc_feature 1..36: note = oligonucleotide primer
  source 1..36: mol_type = other DNA
tcgaaacatatccaggcatgccttctgaaagggaag

SEQ ID NO: 19 - DNA, 27 bp, synthetic construct
  misc_feature 1..27: note = oligonucleotide primer
  source 1..27: mol_type = other DNA
tcaacgccgggaggccttcctgtaccg

SEQ ID NO: 20 - protein, 19 aa, synthetic construct
  REGION 1..19: note = Synthetic peptide
  SITE 5: note = MISC_FEATURE - phosphorylated serine
FRRHSWISFDVDNGTSAGR

SEQ ID NO: 21 - DNA, 5910 bp, Homo sapiens
  source 1..5910: mol_type = genomic DNA
agatgcaggggcgcaaacgccaaaggagaccaggctgtaggaagagaagggcagagcgccggacagctcggcccgctccccgtcctttggggccgcggctggggaactacaaggcccagcaggcagctgcagggggcggaggcggaggagggaccagcgcgggtgggagtgagagagcgagccctcgcgccccgccggcgcatagcgctcggagcgctcttgcggccacaggcgcggcgtcctcggcggcgggcggcagctagcgggagccgggacgccggtgcagccgcagcgcgcggaggaacccgggtgtgccgggagctgggcggccacgtccggacgggaccgagacccctcgtagcgcattgcggcgacctcgccttccccggccgcgagcgcgccgctgcttgaaaagccgcggaacccaaggacttttctccggtccgagctcggggcgccccgcagggcgcacggtacccgtgctgcagtcgggcacgccgcggcgccggggcctccgcagggcgatggagcccggtctgcaaggaaagtgaggcgccgccgctgcgttctggaggaggggggcacaaggtctggagaccccgggtggcggacgggagccctccccccgccccgcctccggggcaccagctccggctccattgttcccgcccgggctggaggcgccgagcaccgagcgccgccgggagtcgagcgccggccgcggagctcttgcgaccccgccaggacccgaacagagcccgggggcggcgggccggagccggggacgcgggcacacgcccgctcgcacaagccacggcggactctcccgaggcggaacctccacgccgagcgagggtcagtttgaaaaggaggatcgagctcactgtggagtatccatggagatgtggagccttgtcaccaacctctaactgcagaactgggatgtggagctggaagtgcctcctcttctgggctgtgctggtcacagccacactctgcaccgctaggccgtccccgaccttgcctgaacaagcccagccctggggagcccctgtggaagtggagtccttcctggtccaccccggtgacctgctgcagcttcgctgtcggctgcgggacgatgtgcagagcatcaactggctgcgggacggggtgcagctggcggaaagcaaccgcacccgcatcacaggggaggaggtggaggtgcaggactccgtgcccgcagactccggcctctatgcttgcgtaaccagcagcccctcgggcagtgacaccacctacttctccgtcaatgtttcagttcccatagatgctctcccctcctcggaggatgatgatgatgatgatgactcctcttcagaggagaaagaaacagataacaccaaaccaaaccccgtagctccatattggacatccccagaaaagatggaaaagaaattgcatgcagtgccggctgccaagacagtgaagttcaaatgcccttccagtgggaccccaaaccccacactgcgctggttgaaaaatggcaaagaattcaaacctgaccacagaattggaggctacaaggtccgttatgccacctggagcatcataatggactctgtggtgccctctgacaagggcaactacacctgcattgtggagaatgagtacggcagcatcaaccacacataccagctggatgtcgtggagcggtcccctcaccggcccatcctgcaagcagggttgcccgccaacaaaacagtggccctgggtagcaacgtggagttcatgtgtaaggtgtacagtgacccgcagccgcacatccagtggctaaagcacatcgaggtgaatgggagcaagattggcccagacaacctgccttatgtccagatcttgaagcattcggggattaatagctcggatgcggaggtgctgaccctgttcaatgtgacagaggcccagagcggggagtatgtgtgtaaggtttccaattatattggtgaagctaaccagtctgcgtggctcactgtcaccagacctgtggcaaaagccctggaagagaggccggcagtgatgacctcgcccctgtacctggagatcatcatctattgcacaggggccttcctcatctcctgcatggtggggtcggtcatcgtctacaagatgaagagtggtaccaagaagagtgacttccacagccagatggctgtgcacaagctggccaagagcatccctctgcgcagacaggtaacagtgtctgctgactccagtgcatccatgaactctggggttcttctggttcggccatcacggctctcctccagtgggactcccatgctagcaggggtctctgagtatgagcttcccgaagaccctcgctgggagctgcctcgggacagactggtcttaggcaaacccctgggagagggctgctttgggcaggtggtgttggcagaggctatcgggctggacaaggacaaacccaaccgtgtgaccaaagtggctgtgaagatgttgaagtcggacgcaacagagaaagacttgtcagacctgatctcagaaatggagatgatgaagatgatcgggaagcataagaatatcatcaacctgctgggggcctgcacgcaggatggtcccttgtatgtcatcgtggagtatgcctccaagggcaacctgcgggagtacctgcaggcccggaggcccccagggctggaatactgctacaaccccagccacaacccagaggagcagctctcctccaaggacctggtgtcctgcgcctaccaggtggcccgaggcatggagtatctggcctccaagaagtgcatacaccgagacctggcagccaggaatgtcctggtgacagaggacaatgtgatgaagatagcagactttggcctcgcacgggacattcaccacatcgactactataaaaagacaaccaacggccgactgcctgtgaagtggatggcacccgaggcattatttgaccggatctacacccaccagagtgatgtgtggtctttcggggtgctcctgtgggagatcttcactctgggcggctccccataccccggtgtgcctgtggaggaacttttcaagctgctgaaggagggtcaccgcatggacaagcccagtaactgcaccaacgagctgtacatgatgatgcgggactgctggcatgcagtgccctcacagagacccaccttcaagcagctggtggaagacctggaccgcatcgtggccttgacctccaaccaggagtacctggacctgtccatgcccctggaccagtactcccccagctttcccgacacccggagctctacgtgctcctcaggggaggattccgtcttctctcatgagccgctgcccgaggagccctgcctgccccgacacccagcccagcttgccaatggcggactcaaacgccgctgactgccacccacacgccctccccagactccaccgtcagctgtaaccctcacccacagcccctgctgggcccaccacctgtccgtccctgtcccctttcctgctggcaggagccggctgcctaccaggggccttcctgtgtggcctgccttcaccccactcagctcacctctccctccacctcctctccacctgctggtgagaggtgcaaagaggcagatctttgctgccagccacttcatcccctcccagatgttggaccaacacccctccctgccaccaggcactgcctggagggcagggagtgggagccaatgaacaggcatgcaagtgagagcttcctgagctttctcctgtcggtttggtctgttttgccttcacccataagcccctcgcactctggtggcaggtgccttgtcctcagggctacagcagtagggaggtcagtgcttcgtgcctcgattgaaggtgacctctgccccagataggtggtgccagtggcttattaattccgatactagtttgctttgctgaccaaatgcctggtaccagaggatggtgaggcgaaggccaggttgggggcagtgttgtggccctggggcccagccccaaactgggggctctgtatatagctatgaagaaaacacaaagtgtataaatctgagtatatatttacatgtctttttaaaagggtcgttaccagagatttacccatcgggtaagatgctcctggtggctgggaggcatcagttgctatatattaaaaacaaaaaagaaaaaaaaggaaaatgtttttaaaaaggtcatatattttttgctacttttgctgttttatttttttaaattatgttctaaacctattttcagtttaggtccctcaataaaaattgctgctgcttcatttatctatgggctgtatgaaaagggtgggaatgtccactggaaagaagggacacccacgggccctggggctaggtctgtcccgagggcaccgcatgctcccggcgcaggttccttgtaacctcttcttcctaggtcctgcacccagacctcacgacgcacctcctgcctctccgctgcttttggaaagtcagaaaaagaagatgtctgcttcgagggcaggaaccccatccatgcagtagaggcgctgggcagagagtcaaggcccagcagccatcgaccatggatggtttcctccaaggaaaccggtggggttgggctggggagggggcacctacctaggaatagccacggggtagagctacagtgattaagaggaaagcaagggcgcggttgctcacgcctgtaatcccagcactttgggacaccgaggtgggcagatcacttcaggtcaggagtttgagaccagcctggccaacttagtgaaaccccatctctactaaaaatgcaaaaattatccaggcatggtggcacacgcctgtaatcccagctccacaggaggctgaggcagaatcccttgaagctgggaggcggaggttgcagtgagccgagattgcgccattgcactccagcctgggcaacagagaaaacaaaaaggaaaacaaatgatgaaggtctgcagaaactgaaacccagacatgtgtctgccccctctatgtgggcatggttttgccagtgcttctaagtgcaggagaacatgtcacctgaggctagttttgcattcaggtccctggcttcgtttcttgttggtatgcctccccagatcgtccttcctgtatccatgtgaccagactgtatttgttgggactgtcgcagatcttggcttcttacagttcttcctgtccaaactccatcctgtccctcaggaacggggggaaaattctccgaatgtttttggttttttggctgcttggaatttacttctgccacctgctggtcatcactgtcctcactaagtggattctggctcccccgtacctcatggctcaaactaccactcctcagtcgctatattaaagcttatattttgctggattactgctaaatacaaaagaaagttcaatatgttttcatttctgtagggaaaatgggattgctgctttaaatttctgagctagggattttttggcagctgcagtgttggcgactattgtaaaattctctttgtttctctctgtaaatagcacctgctaacattacaatttgtatttatgtttaaagaaggcatcatttggtgaacagaactaggaaatgaatttttagctcttaaaagcatttgctttgagaccgcacaggagtgtctttccttgtaaaacagtgatgataatttctgccttggccctaccttgaagcaatgttgtgtgaagggatgaagaatctaaaagtcttcataagtccttgggagaggtgctagaaaaatataaggcactatcataattacagtgatgtccttgctgttactactcaaatcacccacaaatttccccaaagactgcgctagctgtcaaataaaagacagtgaaattgacctga

SEQ ID NO: 22 - protein, 825 aa, Homo sapiens
MWSWKCLLFWAVLVTATLCTARPSPTLPEQAQPWGAPVEVESFLVHPGDLLQLRCRLRDDVQSINWLRDGVQLAESNRTRITGEEVEVQDSVPADSGLYACVTSSPSGSDTTYFSVNVSVPIDALPSSEDDDDDDDSSSEEKETDNTKPNPVAPYWTSPEKMEKKLHAVPAAKTVKFKCPSSGTPNPTLRWLKNGKEFKPDHRIGGYKVRYATWSIIMDSVVPSDKGNYTCIVENEYGSINHTYQLDVVERSPHRPILQAGLPANKTVALGSNVEFMCKVYSDPQPHIQWLKHIEVNGSKIGPDNLPYVQILKHSGINSSDAEVLTLFNVTEAQSGEYVCKVSNYIGEANQSAWLTVTRPVAKALEERPAVMTSPLYLEIIIYCTGAFLISCMVGSVIVYKMKSGTKKSDFHSQMAVHKLAKSIPLRRQVTVSADSSASMNSGVLLVRPSRLSSSGTPMLAGVSEYELPEDPRWELPRDRLVLGKPLGEGCFGQVVLAEAIGLDKDKPNRVTKVAVKMLKSDATEKDLSDLISEMEMMKMIGKHKNIINLLGACTQDGPLYVIVEYASKGNLREYLQARRPPGLEYCYNPSHNPEEQLSSKDLVSCAYQVARGMEYLASKKCIHRDLAARNVLVTEDNVMKIADFGLARDIHHIDYYKKTTNGRLPVKWMAPEALFDRIYTHQSDVWSFGVLLWEIFTLGGSPYPGVPVEELFKLLKEGHRMDKPSNCTNELYMMMRDCWHAVPSQRPTFKQLVEDLDRIVALTSNQEYLDLSMPLDQYSPSFPDTRSSTCSSGEDSVFSHEPLPEEPCLPRHPAQLANGGLKRR

SEQ ID NO: 23 - DNA, 716 bp, Homo sapiens
  source 1..716: mol_type = genomic DNA
agcaggctcagaaggtccagaaatcaggggaaggagacccctatctgtccttcttctggaagagctggaaaggaagtctgctcaggaaataaccttggaagatggtggccacgaagacctttgctctgctgctgctgtccctgttcctggcagtgggactaggagagaagaaagagggtcacttcagcgctctcccctccctgcctgttggatctcatgctaaggtgagcagccctcaacctcgaggccccaggtacgcggaagggactttcatcagtgactacagtattgccatggacaagattcaccaacaagactttgtgaactggctgctggcccaaaaggggaagaagaatgactggaaacacaacatcacccagagggaggctcgggcgctggagctggccagtcaagctaataggaaggaggaggaggcagtggagccacagagctccccagccaagaaccccagcgatgaagatttgctgcgggacttgctgattcaagagctgttggcctgcttgctggatcagacaaacctctgcaggctcaggtctcggtgactctgaccacacccagctcaggactggattctgcccttcacttagcacctgcctcagccccactccagaatagccaagagaacccaaaccaataaagtttatgctaagtcgagcccattgtgaaaatttattaaaatgactactgagcactaa

SEQ ID NO: 24 - protein, 153 aa, Homo sapiens
MVATKTFALLLLSLFLAVGLGEKKEGHFSALPSLPVGSHAKVSSPQPRGPRYAEGTFISDYSIAMDKIHQQDFVNWLLAQKGKKNDWKHNITQREARALELASQANRKEEEAVEPQSSPAKNPSDEDLLRDLLIQELLACLLDQTNLCRLRSR

SEQ ID NO: 25 - DNA, 264 bp, Heloderma suspectum
  source 1..264: mol_type = genomic DNA
atgaaaatcatcctgtggctgtgtgtttttgggctgttccttgcaactttattccctatcagctggcaaatgcctgttgaatctgggttgtcttctgaggattctgcaagctcagaaagctttgcttcgaagattaagcgacatggtgaaggaacatttaccagtgacttgtcaaaacagatggaagaggaggcagtgcggttatttattgagtggcttaagaacggaggaccaagtagcggggcacctccgccatcgggttaa

SEQ ID NO: 26 - protein, 39 aa, Heloderma suspectum
HGEGTFTSDLSKQMEEEAVRLFIEWLKNGGPSSGAPPPS

SEQ ID NO: 27 - DNA, 874 bp, synthetic construct
  misc_feature 1..874: note = Synthetic nucleic acid
  source 1..874: mol_type = other DNA
ctctttccacatgacggcctttgtggtgggtggcagattgccctgaggcctcgcaaaatgctaggctttcacaatgtcactgactgacagccaggcccagcacagtcttggtgtgattgtggggctaaagttattccaccttgtgcaatagctacagcagctttaagaattcagggcctttttaacttgccaagccccacaccactccaggaacttccccacaccccagttctcagaattcatgtgcaaggtctttcctaaatccagggtccaggtcagagagtggaggatgtgctctatttcttacctgattgcagacccctctgacagtgctcccttctgaagcactcactgtctgaacgtacacagtctcagactaatcatgcacagtgagcaagactgtggtgtgataattggcgtccctgacttattagggcaaatctatgggagggggagacctcctggaccactgagcaattaattcatttacattaggaagtttctccgtcagatgcaggaaaaaaatcttgttttcctgctgtggttttgacttttgccccatcttctgttgctgttgtaggaggcaaaataagggtcaaggcctggaaacacaagtgctttgactgaagctccacttggcttccgaagcccaagctgggttgtaccaggttccctagggtgcaggctgtgggcaactgccagggacatgtgcctgcccaccggcctctggccctcactgagttggccaatgggaaatgacaattgtgaggtggggactgcctgcccccgtgagtaccaggctgttgaggctgggccatctcctcctcacttccattctgactgcagtctgtggttctgattccataccagaggg

SEQ ID NO: 28 - DNA, 100 bp, synthetic construct
  misc_feature 1..100: note = Synthetic nucleic acid
  source 1..100: mol_type = other DNA
caaacaccattgtcacactccatcaccaaacaccattgtcacactccaattacaaacaccattgtcacactccatcaccaaacaccattgtcacactcca

SEQ ID NO: 29 - protein, 140 aa, synthetic construct
FNLPPGHYKKPKLLYCSDGGHFLRILPDGTVEGTRDRSDQHIQLQLSAESVGEVYIKSTETGQCLAMDTDGLLYGSQTPNEECLLLERLGENHYNTYISKRHAEKNWFVGLKKSGSCKRGPRTHYGQKAILFLPLPVSSD

SEQ ID NO: 30 - protein, 140 aa, synthetic construct
FNLPPGNYKEPKPLYCSNGGHFLRILPDGTVDGTRDRSDQHIQPQLSAESVGEVYIKSTETGQYLAMDTDGLLYGSQTPNEECLFLERLEENHYNTYTSKKHAEKNWFVGLKQNGSCKRGPRTHYGQKAILFLPLPVSSD

SEQ ID NO: 31 - protein, 140 aa, synthetic construct
FNLPPGNYKKPKLLYCSDRGHFLRILPDGTVDGTRDRSDQHIQLQLSAESVGEVYIKSTETGQYLAMDTDGLLYGSQTPNEECLFLERLEENHYNTYISKKHAEKNWFVGLKKNGSCERGPRTHYGQKAILFLPLPVSSD

SEQ ID NO: 32 - protein, 140 aa, synthetic construct
FNLPPGNYKKPKLLYCSNGGHFLRILPDGTVDGTRDRSDQHIQLQLSAESVGEVYIKSTETGQYLAMDTDGLLYGPQTPNEECLFLERLEENHYNTYISKKHAEKNWFVGLKKNGSCKRGPRTHYGQKAILFLPLPVSSD

SEQ ID NO: 33 - protein, 15 aa, synthetic construct
NTFPFRRHSWICFDY

SEQ ID NO: 34 - protein, 15 aa, synthetic construct
NNFPFRRHSWICFDY

SEQ ID NO: 35 - protein, 11 aa, synthetic construct
HSQRRESFLYR

SEQ ID NO: 36 - protein, 5 aa, synthetic construct
QRRES

SEQ ID NO: 37 - protein, 5 aa, unidentified
FRRHS